SEQUENCE LISTING (55 sequences)

SEQ ID NO: 1 - protein, 585 aa, Homo sapiens [sequence omitted]

SEQ ID NO: 2 - protein, 584 aa, Mus musculus
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 3 - protein, 129 aa, Homo sapiens [sequence omitted]

SEQ ID NO: 4 - protein, 120 aa, Mus musculus
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 5 - protein, 722 aa, Artificial Sequence
  Artificial Polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 6 - protein, 714 aa, Artificial Sequence
  Artificial Polypeptide
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSGGGSMHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 7 - protein, 176 aa, Homo sapiens
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 8 - protein, 165 aa, Mus musculus
AASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 9 - protein, 769 aa, Artificial Sequence
  Artificial Polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 10 - protein, 757 aa, Artificial Sequence
  Artificial Polypeptide
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGGSAASVDTLSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 11 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 12 - protein, 160 aa, Mus musculus
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 13 - protein, 753 aa, Artificial Sequence
  Artificial Polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 14 - protein, 752 aa, Artificial Sequence
  Artificial Polypeptide
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGSGGGSSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 15 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 16 - protein, 152 aa, Mus musculus
RPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 17 - protein, 115 aa, Homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 18 - protein, 112 aa, Mus musculus
EIPMSTVVKETLTQLSAHRALLTSNETMRLPVPTHKNHQLCIGEIFQGLDILKNQTVRGGTVEMLFQNLSLIKKYIDRQKEKCGEERRRTRQFLDYLQEFLGVMSTEWAMEG

SEQ ID NO: 19 - protein, 178 aa, Homo sapiens
PGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 20 - protein, 179 aa, Mus musculus
MPGPPAGSPRVSSDPRADLDSAVLLTRSLLADTRQLAAQMRDKFPADGDHSLDSLPTLAMSAGTLGSLQLPGVLTRLRVDLMSYLRHVQWLRRAGGPSLKTLEPELGALQARLERLLRRLQLLMSRLALPQAAPDQPVIPLGPPASAWGSIRAAHAILGGLHLTLDWAVRGLLLLKTRL

SEQ ID NO: 21 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 22 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 23 - protein, 215 aa, Homo sapiens
FPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 24 - protein, 209 aa, Homo sapiens
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 25 - protein, 206 aa, Homo sapiens
FPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 26 - protein, 180 aa, Homo sapiens
YTETALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYC

SEQ ID NO: 27 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 209 aa, Homo sapiens; identical to SEQ ID NO: 24

SEQ ID NO: 29 - protein, 193 aa, Homo sapiens
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 30 - protein, 206 aa, Homo sapiens
ALVALSQPRVQCHASRYPVAVDCSWTPLQAPNSTRSTSFIATYRLGVATQQQSQPCLQRSPQASRCTIPDVHLFSTVPYMLNVTAVHPGGASSSLLAFVAERIIKPDPPEGVRLRTAGQRLQVLWHPPASWPFPDIFSLKYRLRYRRRGASHFRQVGPIEATTFTLRNSKPHAKYCIQVSAQDLTDYGKPSDWSLPGQVESAPHKP

SEQ ID NO: 31 - protein, 154 aa, Homo sapiens
VLSGALCFRMKDSALKVLYLHNNQLLAGGLHAGKVIKGEEISVVPNRWLDASLSPVILGVQGGSQCLSCGVGQEPTLTLEPVNIMELYLGAKESKSFTFYRRDMGLTSSFESAAYPGWFLCTVPEADQPVRLTQLPENGGWNAPITDFYFQQCD

SEQ ID NO: 32 - protein, 154 aa, Mus musculus
VLSGALCFRMKDSALKVLYLHNNQLLAGGLHAEKVIKGEEISVVPNRALDASLSPVILGVQGGSQCLSCGTEKGPILKLEPVNIMELYLGAKESKSFTFYRRDMGLTSSFESAAYPGWFLCTSPEADQPVRLTQIPEDPAWDAPITDFYFQQCD

SEQ ID NO: 33 - protein, 173 aa, Homo sapiens
VHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 34 - protein, 171 aa, Mus musculus
VPSSAKVNARAPEKFSIRDQDQKVLVLSDETLIAVPNKPYTVPETFFVLASHTSSSHEGSPILLAVSKGELCLCCDKDEEQSKPSLQLKKNELMKLATQKEKVRLPFVFYRAQVGSCCTLESAAHPGWFVCTSRNSGAPVEVTDTSGEGKLMEFSFQQVSETEMSPSEVSI

SEQ ID NO: 35 - protein, 165 aa, Homo sapiens
SYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 36 - protein, 161 aa, Mus musculus
INYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 37 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 38 - protein, 112 aa, Mus musculus
ALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 39 - protein, 9 aa, Artificial Sequence
  Artificial Polypeptide
YPYDVPDYA

SEQ ID NO: 40 - protein, 8 aa, Artificial Sequence
  Artificial Polypeptide
DYKDDDDK

SEQ ID NO: 41 - protein, 10 aa, Artificial Sequence
  Artificial Polypeptide
EQKLISEEDL

SEQ ID NO: 42 - protein, 6 aa, Artificial Sequence
  Artificial Polypeptide
HHHHHH

SEQ ID NO: 43 - protein, 4 aa, Artificial Sequence
  Artificial Polypeptide
GGGS

SEQ ID NO: 44 - protein, 4 aa, Artificial Sequence
  Artificial Polypeptide
GGSG

SEQ ID NO: 45 - protein, 5 aa, Artificial Sequence
  Artificial Polypeptide
GGSGG

SEQ ID NO: 46 - protein, 5 aa, Artificial Sequence
  Artificial Polypeptide
GSGSG

SEQ ID NO: 47 - protein, 5 aa, Artificial Sequence
  Artificial Polypeptide
GSGGG

SEQ ID NO: 48 - protein, 5 aa, Artificial Sequence
  Artificial Polypeptide
GGGSG

SEQ ID NO: 49 - protein, 5 aa, Artificial Sequence
  Artificial Polypeptide
GSSSG

SEQ ID NO: 50 - protein, 8 aa, Artificial Sequence
  Artificial Polypeptide
GGGSGGGS

SEQ ID NO: 51 - protein, 11 aa, Artificial Sequence
  Artificial Polypeptide
DICLPRWGCLW

SEQ ID NO: 52 - protein, 773 aa, artificial sequence
  synthetic polypeptide
METDTLLLWVLLLWVPGSTGDGEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSGGGSSIQGTSLLTQSPASLSTYNDQSVSFVLENGCYVINVDDSGKDQEQDQVLLRYYESPCPASQSGDGVDGKKLMVNMSPIKDTDIWLHANDKDYSVELQRGDVSPPEQAFFVLHKKSSDFVSFECKNLPGTYIGVKDNQLALVEEKDESCNNIMFKLSKI

SEQ ID NO: 53 - protein, 1027 aa, artificial sequence
  synthetic polypeptide
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGSGGGSGGGSGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 54 - protein, 778 aa, artificial sequence
  synthetic polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSGGGSGGGSGGGSGGGSVHTSPKVKNLNPKKFSIHDQDHKVLVLDSGNLIAVPDKNYIRPEIFFALASSLSSASAEKGSPILLGVSKGEFCLYCDKDKGQSHPSLQLKKEKLMKLAAQKESARRPFIFYRAQVGSWNMLESAAHPGWFICTSCNCNEPVGVTDKFENRKHIEFSFQPVCKAEMSPSEVSD

SEQ ID NO: 55 - protein, 152 aa, artificial sequence
  synthetic polypeptide
MCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

(19) United States
(12) Patent Application Publication
(10) Pub. No.: US 2024/0052006 A1
(43) Pub. Date: Feb. 15, 2024

(54) ANTI-INFLAMMATORY CYTOKINES AND METHODS OF USE

(71) Applicant: THE UNIVERSITY OF CHICAGO, Chicago, IL (US)

(72) Inventors: Jeffrey A HUBBELL, Chicago, IL (US); Eiji YUBA, Chicago, IL (US); Elyse WATKINS, Chicago, IL (US); Jun ISHIHARA, Chicago, IL (US); Ako ISHIHARA, Chicago, IL (US); Erica BUDINA, Chicago, IL (US); Abigail LAUTERBACH, Chicago, IL (US)

(73) Assignee: THE UNIVERSITY OF CHICAGO, Chicago, IL (US)

(21) Appl. No.: 18/246,512

(22) PCT Filed: Sep. 24, 2021
(86) PCT No.: PCT/US2021/071587

§ 371 Date: Mar. 23, 2023

Related U.S. Application Data
(60) Provisional application No. 63/083,722, filed on Sep. 25, 2020.

(51) Int. Cl. C07K 14/54; A61P 37/02; C12N 15/63

(52) U.S. Cl. CPC C07K 14/54; A61P 37/02; C12N 15/63; C07K 2319/31

(57) ABSTRACT

Aspects of the present disclosure provide methods and compositions for treatment of autoimmune disorders including, for example, multiple sclerosis and rheumatoid arthritis. Also disclosed are methods for promoting wound healing. Certain aspects are directed to antiinflammatory cytokines operatively linked to an albumin protein. Further aspects relate to methods for delivering an anti-inflammatory cytokine to a lymph node.

### CROSS-REFERENCE TO RELATED APPLICATIONS

This application claims the benefit of priority of U.S. Provisional Patent Application No. 63/083,722 filed Sep. 25, 2020, which is hereby incorporated by reference in its entirety.

### BACKGROUND

### I. Sequence Listing

The instant application contains a Sequence Listing which has been submitted in ASCII format and is hereby incorporated by reference in its entirety. Said ASCII copy, created on Sep. 23, 2021, is named ARCDP0711WO.txt and is 120,558 bytes in size.

### II. Field of the Invention

This invention relates to at least the fields of molecular biology, immunology, and medicine.

### III. Background

Multiple sclerosis (MS) is a potentially disabling autoimmune disease that affects millions globally. Autoreactive immune cells home to the central nervous system (CNS) and cause demyelination and consequently focal damage to white matter¹. Lymphocytes and macrophages that have infiltrated into the CNS cause axonal damage. Recent studies have shown that Th17 cells, activated in the secondary lymphoid organs (SLOs), migrate to the spinal cord and brain and play a crucial role in the disease development and severity of MS^{2, 3}. Thus, inhibition of lymphocyte migration to the CNS and inducing an immune-suppressive microenvironment in the SLOs would provide an effective therapy for MS. FTY720 (fingolimod) and anti-integrin α4 antibody (natalizumab) are used in the clinic for treating MS^{4, 5}, sequestering lymphocytes in the LNs and preventing them from reacting with autoantigens in target tissues. Experimental autoimmune encephalomyelitis (EAE) is a widely accepted murine model of MS, reflecting many features of disease progression and developmental mechanism, including lymphocyte migration to the CNS and demyelination.

Rheumatoid arthritis (RA) is an autoimmune disease that is currently controlled through treatment with inhibitors of inflammatory pathways. Pathological features of RA are synovitis and joint destruction, which cause severe pain and joint dysfunction (48, 49). Although the causal antigen for RA has not been fully elucidated, collagen recognition by immune cells plays a key role. During progression of RA, autoantigen-specific T cells, especially Th17 cells, are activated and produce inflammatory cytokines including IL-17. Inflammatory cytokines, such as TNF-α and IL-6, in the joint induce activation of macrophages and neutrophils as mediators of the inflammatory response. These inflammatory cells infiltrate the joints and cause various inflammatory responses including activation of osteoclasts that destroy the bones in the joint (50). The current strategy for RA treatment is symptomatic, and considering that many inflammatory cytokines are involved RA progression, various biological therapeutics, such as antibodies or soluble receptors for TNF-α have been developed and approved for clinical use (51).

Various strategies for treatment of autoimmune disorders, such as MS and RA, have been explored involving the use of anti-inflammatory cytokines. Thus far, such strategies have not been translated to clinical use. There remains a need for compositions and methods for treatment of autoimmune disorders, including MS and RA, involving effective delivery of anti-inflammatory cytokines.

### SUMMARY

Aspects of the present disclosure are directed to compositions comprising an anti-inflammatory cytokine operatively linked to an albumin protein, along with methods of use involving such compositions, including methods for treatment of various conditions including autoimmune or inflammatory conditions.

Aspects of the present disclosure include therapeutic polypeptides, anti-inflammatory polypeptides, anti-inflammatory compositions, pharmaceutical compositions, nucleic acid molecules, vectors, therapeutic cells, methods for treating an autoimmune condition, methods for treating an inflammatory condition, methods for promoting wound healing, methods for treating a subject for multiple sclerosis (MS), methods for treating a subject for rheumatoid arthritis, methods for inhibiting a function of Th17 cells, methods for reducing inflammation in a subject, methods for targeting an anti-inflammatory cytokine to a lymph node, methods for detecting an anti-inflammatory cytokine in a lymph node, methods for diagnosing a subject with an autoimmune or inflammatory condition, methods for targeting a cytokine to a lymph node of a subject, and methods for preventing an autoimmune or inflammatory condition.

Polypeptides of the present disclosure can include at least 1, 2, 3, 4 or more of the following components: an anti-inflammatory cytokine, an albumin protein, an albumin binding protein, a linker, a tag, a label, and an anti-inflammatory molecule, which components may be in any order starting from the N-terminus. Methods of the present disclosure can include at least 1, 2, 3, 4, or more of the following steps: administering a composition to a subject, obtaining a biological sample from a subject, obtaining a lymph sample from a subject, detecting an anti-inflammatory cytokine in a lymph sample from a subject, generating a polypeptide comprising an anti-inflammatory cytokine, attaching an anti-inflammatory cytokine to an albumin protein via a linker, attaching an anti-inflammatory cytokine to an albumin binding protein via a linker, diagnosing a subject for an autoimmune or inflammatory condition, treating a subject for an autoimmune or inflammatory condition, promoting wound healing in a subject, and reducing inflammation in a subject.

Disclosed herein, in some aspects, is a method for treating a subject for an autoimmune or inflammatory condition, the method comprising administering to the subject, by subcutaneous, intradermal, or intramuscular administration, an effective amount of a composition comprising an anti-inflammatory cytokine operatively attached to an albumin protein. Also contemplated are methods for targeting an anti-inflammatory cytokine to a lymph node in a subject by administering an effective amount of a composition comprising an anti-inflammatory cytokine operatively attached to an albumin protein. In some aspects, the condition is multiple sclerosis (MS). The MS may be further defined as primary-progressive MS. In some aspects, the MS comprises secondary-progressive MS. In some aspects, the MS is further defined as relapsing-remitting MS. In some aspects, the MS is further defined as clinically isolated syndrome. In some aspects, the subject is one who is experiencing or one who has experienced an acute attack at a time period of at most 48 hours prior to administration. In some aspects, the MS is late-stage MS. The subject may be one who is defined as having active, not active, worsening, or not worsening MS. A subject with active MS is defined as a subject who is experiencing an episode or MS symptoms and one who has evidence of disease progression. A subject with not active MS is defined as a subject in which the condition is stable, and there is no apparent evidence that the disease is progressing. A subject with worsening MS is defined as one who has a confirmed and notable increase in their disability following a relapse. A subject with not worsening MS is defined as one who experiences a relapse but shows no new or worsened signs of disability. In some aspects, the MS disease is suppressed upon administration of the composition. The suppression may be at least 10, 15, 20, 25, 30, 35, 40, 45, 50, 55, 60, 65, 70, 75, 80, 85, or 90% suppression. In some aspects, demylenation is inhibited upon administration of the composition. The demylenation may be inhibited by at least 10, 15, 20, 25, 30, 35, 40, 45, 50, 55, 60, 65, 70, 75, 80, 85, or 90%.

In some aspects, the anti-inflammatory cytokine is IL-4. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:5. In some aspects, the anti-inflammatory cytokine operatively linked to the albumin protein comprises SEQ ID NO:5. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:6. In some aspects, the anti-inflammatory cytokine operatively linked to the albumin protein comprises SEQ ID NO:6. In some aspects, the anti-inflammatory cytokine is IL-33. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:9. In some aspects, the anti-inflammatory cytokine operatively linked to the albumin protein comprises SEQ ID NO:9. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:10. In some aspects, the anti-inflammatory cytokine operatively linked to the albumin protein comprises SEQ ID NO:10. In some aspects, the condition is arthritis, multiple sclerosis, or scleroderma. In some aspects, the arthritis is rheumatoid arthritis. In some aspects, the anti-inflammatory cytokine is IL-10. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:13. In some aspects, the anti-inflammatory cytokine operatively linked to the albumin protein comprises SEQ ID NO:13. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:14. In some aspects, the anti-inflammatory cytokine operatively linked to the albumin protein comprises SEQ ID NO:14. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:52. In some aspects, the anti-inflammatory cytokine operatively linked to the albumin protein comprises SEQ ID NO:52. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:53. In some aspects, the anti-inflammatory cytokine operatively linked to the albumin protein comprises SEQ ID NO:53. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:54. In some aspects, the anti-inflammatory cytokine operatively linked to the albumin protein comprises SEQ ID NO:54. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:55. In some aspects, the anti-inflammatory cytokine operatively linked to the albumin protein comprises SEQ ID NO:55. In some aspects, the condition is type 1 diabetes, diabetic peripheral neuropathy, psoriasis, inflammatory bowel disease, or Crohn's disease. In some aspects, the condition is acute respiratory distress syndrome (ARDS). It is specifically contemplated that one or more of these conditions may be excluded from an aspect.

Disclosed herein, in some aspects, is a method for promoting wound healing in a subject, the method comprising administering to the subject, by subcutaneous, intradermal, or intramuscular administration, an effective amount of a composition comprising an anti-inflammatory cytokine operatively linked to an albumin protein. In some aspects, the composition increases a rate of healing of a wound on the subject relative to a rate of healing of a wound on a subject that is not administered the composition. In some aspects, the wound is a diabetic ulcer. In some aspects, the anti-inflammatory cytokine is IL-4. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO: SEQ ID NO:5. In some aspects, the anti-inflammatory cytokine operatively linked to the albumin protein comprises SEQ ID NO: SEQ ID NO:5. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO: SEQ ID NO:6. In some aspects, the anti-inflammatory cytokine operatively linked to the albumin protein comprises SEQ ID NO: SEQ ID NO:6. In some aspects, the anti-inflammatory cytokine is IL-33. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9%, sequence identity (or any range derivable therein) to SEQ ID NO: SEQ ID NO:9. In some aspects, the anti-inflammatory cytokine operatively linked to the albumin protein comprises SEQ ID NO: SEQ ID NO:9. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9%, sequence identity (or any range derivable therein) to SEQ ID NO: SEQ ID NO:10. In some aspects, the anti-inflammatory cytokine is IL-10. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:13. In some aspects, the anti-inflammatory cytokine operatively linked to the albumin protein comprises SEQ ID NO:13. In some aspects, the anti-inflammatory cytokine operatively linked to the albumin protein comprises a sequence having or having at least 80, 81, 82, 83, 84, 85, 86, 87, 88, 89, 90, 91, 92, 93, 94, 95, 96, 97, 98, 99, 99.5, or 99.9% sequence identity (or any range derivable therein) to SEQ ID NO:14. In some aspects, the anti-inflammatory cytokine operatively linked to the albumin protein comprises SEQ ID NO:14. In aspects of the disclosure the composition may comprise a hyaluronic acid hydrogel carrier.

Also described is a method for treating or preventing cytokine storm syndrome in a subject comprising administering to the subject an effective amount of a composition comprising an IL-27 operatively linked to an albumin binding polypeptide. In some aspects, the subject has cancer. In some aspects, the subject is being treated with an immunotherapy. In some aspects, the immunotherapy comprises immune checkpoint blockade (ICB) therapy, adoptive T-cell therapy, cytokine therapy, CAR-T cell therapy, activation of co-stimulatory molecules, and combinations thereof. In some aspects, the cancer comprises melanoma. In some aspects, the cancer comprises renal carcinoma. In some aspects, the cancer comprises Stage I, II, III, or IV cancer. In some aspects, the cancer comprises metastatic or recurrent cancer. In some aspects, IL-27 comprises one of SEQ ID NOS:23-26, and combinations and fusions thereof.

In aspects of the disclosure, the dose of albumin-cytokine fusion proteins, such as the fusion of albumin with IL-of 4, IL-5, IL-10, IL-11, IL-23, IL-27, IL-33, IL-35, IL-36ra, IL-37, or IL-38 may be, be at least, or be at most 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg, or any derivable range therein.

The subject may be administered, administered at least, or administered at most 1, 2, 3, 4, 5, 6, 7, 8, 9, 10, 11, 12, 13, 14, 15, 16, 17, 18, 19, or 20 doses (or any derivable range therein) during a specified time period, such as within, at least, or at most, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, weeks, or 1, 2, 3, 4, 5, 6, 7, 8, 9, 10, 11, or 12 months (or any range derivable therein).

In aspects of the disclosure, the dose of albumin-cytokine fusion proteins, such as the fusion of albumin with IL-4, IL-5, IL-10, IL-11, IL-23, IL-27, IL-33, IL-35, IL-36ra, IL-37, or IL-38 is, is at least, or is at most 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg, or any derivable range therein and the subject may be administered, administered at least, or administered at most 1, 2, 3, 4, 5, 6, 7, 8, 9, 10, 11, 12, 13, 14, 15, 16, 17, 18, 19, or 20 doses (or any derivable range therein) during a specified time period, such as within, at least, or at most, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, weeks, or 1, 2, 3, 4, 5, 6, 7, 8, 9, 10, 11, or 12 months (or any range derivable therein).

In aspects of the disclosure, albumin linked to IL-4 is administered to a subject in an amount of 0.4-1.5 mg/kg in one dose per week. In aspects of the disclosure, a subject is administered 1 or 2 doses of albumin linked to IL-4 per week, wherein the dose is 0.4-1.5 mg/kg. In aspects of the disclosure, the subject is administered 1 weekly 0.4-1.5 mg/kg dose of albumin linked to IL-4.

In aspects of the disclosure, albumin linked to IL-33 is administered to a subject in an amount of 0.6-12 mg/kg every other day for a total of three doses per week or in one week. The albumin linked to IL-33 may be given to a subject in an amount of 0.6-12 mg/kg every other day for a total of three doses per week for a period of, a period of at least, or a period of at most, 1, 2, 3, 4, 5, 6, 7, 8, 9, 10, 11, or 12 weeks (or any derivable range therein).

In aspects of the disclosure, albumin linked to IL-4 is administered to a subject in an amount of 0.5-5 mg/kg three times per week. In aspects of the disclosure, albumin linked to IL-4 is administered to a subject in an amount of 0.5-5 mg/kg three times per week for the treatment of diabetes, such as Type 1 diabetes.

Based on animal studies that present efficacy and toxicity data of certain doses of compounds in mice, such as those described in the examples, one skilled in the art can estimate an appropriate dosage for a human. This is described in Nair and Jacob, Journal of Basic and Clinical Pharmacy, Vol. 7, Issue 2, March-May 2016, pages 27-31, which is herein incorporated by reference.

Disclosed herein, in some aspects, is a method for treating a subject for multiple sclerosis, the method comprising administering to the subject an effective amount of a composition comprising IL-4 operatively linked to an albumin protein. In some aspects, the IL-4 is human IL-4 and the albumin protein is human serum albumin.

Disclosed herein, in some aspects, is a method for treating a subject for multiple sclerosis, the method comprising administering to the subject, by subcutaneous, intradermal, or intramuscular administration, an effective amount of a composition comprising IL-33 operatively linked to an albumin protein. In some aspects, the IL-33 is human IL-33 and the albumin protein is human serum albumin.

Disclosed herein, in some aspects, is a method for treating a subject for rheumatoid arthritis, the method comprising administering to the subject, by subcutaneous, intradermal, or intramuscular administration, an effective amount of a composition comprising IL-10 operatively linked to an albumin protein. In some aspects, the IL-10 is human IL-10 and the albumin protein is human serum albumin.

Disclosed herein, in some aspects, is a method for treating a subject for rheumatoid arthritis, the method comprising administering to the subject, by subcutaneous, intradermal, or intramuscular administration, an effective amount of a composition comprising IL-35 operatively linked to an albumin protein. In some aspects, the IL-35 is human IL-35 and the albumin protein is human serum albumin.

Disclosed herein, in some aspects, is a method for promoting wound healing in a subject, the method comprising administering to the subject, by subcutaneous, intradermal, or intramuscular administration, an effective amount of a composition comprising IL-4 operatively linked to an albumin protein. In some aspects, the IL-4 is human IL-4 and the albumin protein is human serum albumin.

Disclosed herein, in some aspects, is a method for promoting wound healing in a subject, the method comprising administering to the subject, by subcutaneous, intradermal, or intramuscular administration, an effective amount of a composition comprising IL-33 operatively linked to an albumin protein. In some aspects, the IL-33 is human IL-33 and the albumin protein is human serum albumin.

Disclosed herein, in some aspects, is a method for inhibiting a function of Th17 cells, the method comprising administering to a subject, by subcutaneous, intradermal, or intramuscular administration, an effective amount of a composition comprising an anti-inflammatory cytokine operatively linked to an albumin protein.

Disclosed herein, in some aspects, is a method for reducing inflammation in a subject, the method comprising administering to the subject, by subcutaneous, intradermal, or intramuscular administration, an effective amount of a composition comprising an anti-inflammatory cytokine operatively linked to an albumin protein.

Disclosed herein, in some aspects, is a method for targeting an anti-inflammatory cytokine to a lymph node of a subject, the method comprising administering to the subject, by subcutaneous, intradermal, or intramuscular administration, a composition comprising the anti-inflammatory cytokine operatively linked to an albumin protein. In some aspects, the subject has an autoimmune or inflammatory condition. In some aspects, the method further comprises identifying the anti-inflammatory cytokine in a lymph node of the subject. In some aspects, the identifying comprises obtaining a lymph sample from the subject. In some aspects, the identifying comprises detecting the presence of the anti-inflammatory cytokine in the lymph sample. In some aspects, the anti-inflammatory cytokine remains in the lymph node at least eight hours after administering the composition to the subject. In some aspects, the anti-inflammatory cytokine remains in the lymph node at least sixteen hours after administering the composition to the subject.

In some aspects, the anti-inflammatory cytokine is IL-4. In some aspects, the anti-inflammatory cytokine is IL-5. In some aspects, the anti-inflammatory cytokine is IL-10. In some aspects, the anti-inflammatory cytokine is IL-11. In some aspects, the anti-inflammatory cytokine is IL-23. In some aspects, the anti-inflammatory cytokine is IL-27. In some aspects, the anti-inflammatory cytokine is IL-33. In some aspects, the anti-inflammatory cytokine is IL-35. In some aspects, the anti-inflammatory cytokine is IL-36ra. In some aspects, the anti-inflammatory cytokine is IL-37. In some aspects, the anti-inflammatory cytokine is IL-36ra. In some aspects, the anti-inflammatory cytokine is IL-38. In some aspects, the anti-inflammatory cytokine is interferon-β. In some aspects, the anti-inflammatory cytokine is TGF-β1.

Disclosed herein, in some aspects, is a method for treating a subject for an autoimmune or inflammatory condition, the method comprising administering to the subject, by subcutaneous, intradermal, or intramuscular administration, an effective amount of a composition comprising an anti-inflammatory cytokine operatively linked to an albumin binding polypeptide. In some aspects, the albumin binding peptide is an anti-albumin antibody. In some aspects, the albumin binding protein comprises a sequence having at least 80, 81, 82, 83, 84, 85, 86, 87, 88, 89, 90, 91, 92, 93, 94, 95, 96, 97, 98, 99, 99.5, or 99.9%, sequence identity (or any range derivable therein) to SEQ ID NO:51. In some aspects, the albumin binding protein comprises SEQ ID NO:51.

In some aspects, the albumin protein is human serum albumin. In some aspects, the albumin protein is mouse serum albumin.

In some aspects, the composition is administered to the subject by subcutaneous administration. In some aspects, the composition is administered to the subject by intradermal administration. In some aspects, the composition is administered to the subject by intramuscular administration. In some aspects, the composition is administered to the subject by intravenous administration. In some aspects, the composition is administered systemically to the subject. In some aspects, the albumin protein is operatively linked to an N-terminus of the anti-inflammatory cytokine. In some aspects, the anti-inflammatory cytokine is covalently linked to the albumin protein. In some aspects, the anti-inflammatory cytokine is covalently linked to the albumin protein via a linker. In some embodiments, the albumin is on the amino terminal side of the cytokine. In some embodiments, the albumin is on the carboxy terminal side of the cytokine.

In some aspects, the albumin protein increases the accumulation of the anti-inflammatory cytokine in lymph nodes of the subject relative to an anti-inflammatory cytokine that is not operatively linked to an albumin protein. In some aspects, the composition decreases a number of Th17 cells in the subject. In some aspects, the composition inhibits a function of Th17 cells in the subject.

In some aspects, the polypeptides and compositions of the disclosure treat one or more symptoms of MS. The symptom may comprise visual changes including double vision, blurry vision, or loss of vision, numbness, tingling or weakness (weakness may range from mild to severe), paralysis, vertigo or dizziness, erectile dysfunction (ED, impotence), pregnancy complications, urinary incontinence (or conversely, Urinary retention), muscle spasticity, in coordination of muscles, tremor, painful involuntary muscle contractions, slurred speech, and/or fatigue. The symptom may be reduced by or by at least 5, 10, 15, 20, 25, 30, 35, 40, 45, 50, 55, 60, 65, 70, 75, 80, 85, 90, 95, or 100%, or any derivable range therein for a time period of or of at least 1, 2, 3, 4, 5, 6, 12, 18, 24 hours or 1, 2, 3, 4, 5, 6, 7, 8, 9, 10, 11, 12, 13, 14 days or 1, 2, 3, 4, 5, 6, 7, 8, 9, 10, 11, 12 weeks, or 1, 2, 3, 4, 5, or 6 months (or any derivable range therein).

In some aspects, the composition is administered to the subject via a pre-filled syringe. In some aspects, the anti-inflammatory cytokine is administered at a dose of between 0.1 mg/kg and 50 mg/kg. In some aspects, the anti-inflammatory cytokine is administered at a dose of at least, at most, or about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/kg, or any range or value derivable therein. In some aspects, the subject was previously treated for the condition. In some aspects, the subject was determined to be resistant to the previous treatment. In some aspects, the subject was not previously treated for the condition.

In some aspects, the method further comprises administering to the subject an additional anti-inflammatory agent. In some aspects, the method does not comprise administering to the subject an additional anti-inflammatory agent. In some aspects, the composition is administered during cessation of a treatment of the subject with an additional anti-inflammatory agent. additional anti-inflammatory agent is fingolimod, interferon-β, dimethyl fumarate, teriflunomide, integrin α4β1, or an anti-αLβ2 antibody. In some aspects, the additional anti-inflammatory agent is an anti-TNFα agent, an anti-IL-6R agent, an anti-IL-6 agent, or a Janus kinase inhibitor.

In some aspects, the method comprises administering a nucleic acid to the subject comprising a sequence encoding for the anti-inflammatory cytokine and the albumin protein. In some aspects, the nucleic acid is a vector. In some aspects, the method comprises administering to the subject a cell comprising the vector. In some aspects, the cell is configured to express the vector.

In some aspects, the method further comprises detecting the anti-inflammatory cytokine in a lymph node of the subject. In some aspects, the detecting comprises obtaining a lymph sample from the subject. In some aspects, the detecting comprises detecting the presence of the anti-inflammatory cytokine in the lymph sample.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments and aspects described herein in the context of the term “comprising” may also be implemented in the context of the term “consisting of” or “consisting essentially of.”

“Individual, “subject,” and “patient” are used interchangeably and can refer to a human or non-human. In some aspects, the subject is a human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect. Included, for example, is the use of albumin attached to an anti-inflammatory cytokine to target the cytokine to the lymph nodes of a subject. As another example, included is the use of albumin attached to an anti-inflammatory cytokine to treat an autoimmune or inflammatory condition.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1D—IL-4 retains activity after fusion of serum albumin (SA). FIG. 1A—Wt IL-4 and SA-IL-4 were analyzed by SDS-PAGE under non-reducing (N) and reducing (R) conditions with Coomassie blue staining. FIG. 1B—SA-IL-4 binding to freshly isolated immune cells from (left) LN and (right) spleen, measured by flow cytometry (n=1). FIG. 1C—Wt IL-4 and SA-IL-4 activity assay. Phosphorylation of STAT6 in the T cells was analyzed by flow cytometry after culturing T cells in vitro with indicated concentrations of wt IL-4 or SA-IL-4 (n=2). FIG. 1D—IL-17 concentration secreted under Th17 differentiation conditions in the presence of wt IL-4 or SA-IL-4, measured by ELISA (n=4). Data are mean±SEM. Two experimental replicates. Statistical analyses were performed using one-way ANOVA with Tukey's test.

FIGS. 2A-2J—SA fusion to IL-4 increased the amount of IL-4 in the secondary lymphoid organs after intravenous injection. FIGS. 2A-2B—Amount of IL-4 in the (FIG. 2A) brachial and lumbar LNs and (FIG. 2B) spleen over time. 40 μg of wt IL-4 or equimolar SA-IL-4 was injected intravenously (i.v.) to naïve mice. LNs and spleen were harvested, and IL-4 amount was tested by ELISA (n=5). FIGS. 2C-2D—Amount of IL-4 in the (FIG. 2C) brachial and lumbar LNs and (FIG. 2D) spleen. 40 μg of wt IL-4 or equimolar SA-IL-4 was injected intraperitoneally (i.p.) or subcutaneously (s.c.) to naïve mice. After 4 hrs, LNs and spleen were harvested, and IL-4 amount was tested by ELISA (n=4). FIGS. 2E-2F—Immunofluorescence images of the lumbar LN, 1 hr after intravenous injection of DyLight594-labeled IL-4 or SA-IL-4. T cells and high endothelial venules (HEVs) were respectively stained by anti-CD3 or anti-PNAd antibodies (n=2). Scale bars represent (FIG. 2E) 200 μm and (FIG. 2F) 100 μm. FIG. 2G—Binding affinity of SA-IL-4 to FcRn measured by SPR. FIG. 2H-2I—SA(P573K)-IL-4 amount in (FIG. 2H) the LNs and (FIG. 2I) spleen was measured 1 hr after injection (n=5). Wt IL-4 and SA-IL-4 data from FIGS. 2A-2B are re-presented. FIG. 2J—Transcytosis assay. SA-IL-4 or SA(P573K)-IL-4 was added in the inserts (apical side), where Human Umbilical Vein Endothelial Cells (HUVECs) were cultured. IL-4 in media from both inserts (apical side) and bottom wells (basal side) were measured by ELISA (n=3). Data are mean±SEM. Two experimental replicates. Statistical analyses were performed using one-way ANOVA with Tukey's test (FIGS. 2H and 2I). For the simple comparison (FIGS. 2C, 2D, and 2J), a two-tailed Student's t-test was used.

FIGS. 3A-3J—SA fusion to IL-4 increases its concentration in various organs and blood plasma after injection. FIGS. 3A-3B—Plasma concentration of (FIG. 3A) i.v. and (FIG. 3B) s.c. injected wt IL-4 or SA-IL-4. Wt IL-4 10 μg or equimolar SA-IL-4 (n=4, each) was injected i.v. in naïve mice. Blood was collected after 1 min to 24 hr, and the plasma concentration of IL-4 was measured by ELISA. FIGS. 3C-3G—IL-4 amount in (FIG. 3C) spinal cord, (FIG. 3D) lung, (FIG. 3E) liver, (FIG. 3F) the LNs, and (FIG. 3G) spleen was measured 4 hr after subcutaneous and intraperitoneal injection of Wt IL-4 40 μg or equimolar SA-IL-4 (wt IL-4: n=4, SA-IL-4: n=6). FIGS. 3H-3I—IL-4 in (FIG. 3H) spinal cord (n=4) and (FIG. 3I) lumbar LN in naïve mice and EAE mice were measured by ELISA 1 hr after s.c. injection of SA-IL-4 (40 μg, IL-4 based). (naive: n=4, EAE: n=3) FIG. 3J—Binding affinity of mouse plasma-derived SA (without IL-4) to FcRn measured by SPR. Data are mean±SEM. For FIGS. 3H and 3J, a two-tailed Student's t-test was used. The experiment was performed once.

FIGS. 4A-4C—SA(P573K) mutation to SA-IL-4 decreases blood concentration and abolished FcRn binding. FIG. 4A—SA(P573K)-SA-IL-4 was analyzed by SDS-PAGE under non-reducing conditions with Coomassie blue staining. FIG. 4B—Binding affinity of SA(P573K)-IL-4 and FcRn measured by SPR. The binding affinity could not be determined. 1^{st} and 2^{nd} mean the 62.5 nM concentration was tested twice to validate the variability. FIG. 4C—Mice were injected with 40 μg of wt IL-4, SA-IL-4, or SA(P573K)-IL-4 i.v. After 1 hr, blood was collected and IL-4 concentration in the plasma was determined by ELISA (n=5). Data are mean±SEM. Two experimental replicates.

FIGS. 5A-5D—SA-IL-4 prevents EAE disease progression and development in the acute phase. Disease progression (FIG. 5A) and body weight change (FIG. 5B) in C57BL/6 myelin oligodendrocyte glycoprotein (MOG)₃₅₋₅₅ experimental autoimmune encephalomyelitis (EAE) mice injected every other day for 10 days from day 8 after immunization with phosphate-buffered saline (PBS) intraperitoneally (i.p.), wt IL-4 10 μg i.p., or SA-IL-4 10 μg molar equivalent i.p or subcutaneously (s.c.), or dosed with FTY720 1 mg/kg by oral administration daily. n=7 per group. Number of mice developed EAE symptoms is shown in the figure. FIG. 5C—Representative histology of spinal cord. Myelin expression was detected by immunohistochemistry with anti-myelin basic protein antibody (brown). Arrows indicate demyelination. n=7 per group. Graph represents % of mice showing demyelination in each treatment group by blinded pathology analysis. FIG. 5D—Disease progression in MOG₃₅₋₅₅ induced EAE mice injected every other day for 6 days from day 8 after immunization with PBS, SA-IL-4 and SA(P573K)-IL-4 10 μg molar equivalent i.p. n=6 per group. Two experimental replicates. Data are mean±SEM. Statistical analyses were performed using one-way ANOVA with Tukey's test.

FIG. 6—Subcutaneous injection of wt IL-4 does not show significant therapeutic effect. Shown is disease progression in C57BL/6 MOG₃₅₋₅₅ EAE mice injected s.c. every other day for 16 days from day 8 after immunization with PBS, 10 μg wt IL-4 or equimolar SA-IL-4. Data are mean±SEM, n=7. The experiment was performed once. Statistical analyses were performed using one-way ANOVA with Tukey's test.

FIG. 7—Long-term treatment of SA-IL-4 suppresses EAE disease development and progression. Shown is disease progression in C57BL/6 MOG₃₅₋₅₅ EAE mice injected i.p. every other day for 16 days from day 8 after immunization with PBS or SA-IL-4 (10 μg on an IL-4 basis) (n=8). The number of mice that developed EAE per total mice in each treatment group is indicated. To see the long-term effect SA-IL-4 administration, mice were monitored until day 24. Data are mean±SEM. Two experimental replicates. Statistical analyses were performed using a two-tailed Student's t-test. **P<0.01.

FIGS. 8A-8B—SA-IL-4 did not affect the number of macrophages and dendritic cells in the spinal cord and draining LN. Mice were injected with wt IL-4, SA-IL-4, or PBS i.p. or SA-IL-4 s.c. every other day for 10 days from day 8 after immunization. FTY720 1 mg/kg body weight was administered orally every day from day 8 after immunization. 17 days after immunization, cells from the draining LN (dLN) and spinal cord were isolated and analyzed by flow cytometry. The frequencies of (FIG. 8A) F4/80⁺ macrophage within CD11b⁺ cells, and (FIG. 8B) CD11b⁺ CD11c⁺ DCs within CD45⁺ cells were analyzed. Data are mean±SEM (n=7). The experiment was performed once. Statistical analyses were performed using one-way ANOVA with Tukey's test.

FIGS. 9A-9I—SA-IL-4 treatment inhibits leukocyte infiltration to the spinal cord and induces immune suppressive cells in the draining LN. Mice were injected with wt IL-4, SA-IL-4, or PBS i.p. or SA-IL-4 s.c. every other day for 10 days from day 8 after immunization, or FTY720 1 mg/kg body weight was administered orally every day from day 8 after immunization. 17 days after immunization, cells from the draining LN and spinal cord were isolated and analyzed by flow cytometry. FIGS. 9A-9C—The frequencies of (FIG. 9A) CD45⁺ leukocytes and (FIG. 9B) RoRγt⁺ Th17 cells within live cells in the spinal cord and (FIG. 9C) Ly6G⁺Ly6C⁺ G-MDSCs. IL-4 i.p., n=6; other groups, n=7. FIGS. 9D-9I—In the lumbar draining LN (dLN), frequencies of (FIG. 9D) Ly6G⁺Ly6C⁺ G-MDSCs within CD11b⁺ CD45⁺ cells, (FIG. 9E) Ly6G⁻Ly6C⁺ M-MDSCs within CD11b⁺ CD45⁺ cells, (FIG. 9F) RoRγt⁺ Th17 cells within CD4⁺CD3⁺ T cells, (FIG. 9G) CD86+M1 macrophages within F4/80⁺ CD11b⁺ macrophages, (FIG. 9H) CD206⁺ M2 macrophages within F4/80⁺ CD11b⁺ macrophages, and (FIG. 9I) B220⁺ B cells within CD11b⁺ CD45⁺ cells were analyzed. (For all groups, n=7). Data are mean±SEM. The experiment was performed once. Statistical analyses were performed using one-way ANOVA with Tukey's test.

FIGS. 10A-10R—SA-IL-4 treatment activates the PD-1/PD-L1 axis and decreases integrin and cytokine expression in T cells. MOG₃₅₋₅₅-induced EAE mice were injected with PBS, wt IL-4 or SA-IL-4 s.c. on days 8, 10 and 12 after immunization. For PBS treated group, n=7, for all others n=6. The spinal cord and spleen were isolated on day 13 and immune cells were analyzed (FIGS. 10A-10I). Frequencies of (FIG. 10A) Tetramer⁺ (recognizing MOG₃₅₋₅₅) cells within CD4⁺ T cells in the spinal cord. In the spleen, (FIG. 10B) αLβ2 integrin⁺ cells within Tetramer⁺ CD4⁺ T cells, (FIG. 10C) α4β1 integrin⁺ cells within Tetramer⁺ CD4⁺ T cells, (FIG. 10D) αLβ2 integrin⁺ cells within CD8⁺ T cells, and (FIG. 10E) α4β1 integrin⁺ cells within CD8⁺ T cells, are shown. (FIG. 10F) Mean fluorescence intensity (MFI) of PD-1 of central memory (CM) CD44⁺CD62L⁺CD4⁺ T cells, (FIG. 10G) MFI of PD-1 of CM CD44⁺CD62L⁺CD8⁺ T cells, (FIG. 10H) MFI of PD-L1 of Ly6C⁺Ly6G⁻CD11b⁺ M-MDSC, (FIG. 10I) frequency of PD-L1⁺ of Ly6C⁺Ly6G⁻CD11b⁺ M-MDSC, (FIG. 10J) MFI of PD-L1 of Ly6C⁺Ly6G⁺CD11b⁺ G-MDSC, (FIG. 10K) frequency of PD-L1⁺ of Ly6C⁺Ly6G⁺CD11b⁺ G-MDSC, (FIG. 10L) frequency of IL-23R⁺ cells within Tetramer⁺ CD4⁺ T cells, and (FIG. 10M) FoxP3⁺CD25⁺ Treg cells within Tetramer⁺ CD4⁺ T cells. FIGS. 10N-10P—Splenocytes were cultured in vitro in the presence of MOG protein for 3 days. (FIG. 10N) IL-17A, (FIG. 10O) IFNγ and (FIG. 10P) GM-CSF concentrations in the culture media were analyzed by ELISA. FIGS. 10Q-10R—Splenocytes were cultured ex vivo in the presence of MOG₃₅₋₅₅ peptide for 6 h. Cytokine expression within CD4⁺ T cells was characterized by flow cytometry. Data are mean±SEM. The experiment was performed once. Statistical analyses were performed using one-way ANOVA with Tukey's test.

FIGS. 11A-11L—SA-IL-4 treatment in the chronic phase of EAE decreases the clinical score and prevents immune cell infiltration to the spinal cord. EAE was induced in C57BL/6 mice using MOG₃₅₋₅₅. PBS, wt IL-4 or SA-IL-4 was injected i.p. every other day for 10 days from day 21 after immunization. Disease progression (FIG. 11A) and body weight change (FIG. 11B) are shown (n=6). FIGS. 11C-11D—PBS, wt IL-4 or SA-IL-4 was injected s.c. every other day for 12 days from day 21 after immunization. Disease progression (FIG. 11C) and body weight change (FIG. 11D) are shown (n=8 for PBS and SA-IL-4; n=7 for other treatment groups). FIGS. 11E-11H—On day 34, the spinal cord and spleen were collected and immune cells were analyzed by flow cytometry. Graphs represent frequencies of (FIG. 11E) CD45⁺ cells within live cells in spinal cord, (FIG. 11F) CD4⁺CD3⁺CD45⁺ T cells within live cells in spinal cord, (FIG. 11G) Tetramer⁺ (recognizing MOG₃₅₋₅₅) RoRγt⁺CD4⁺ Th17 cells within live cells in spinal cord, (FIG. 11H) IL-23R⁺ cells within Tetramer⁺ CD4⁺ cells in spleen. FIGS. 11I-11J—Splenocytes were cultured in vitro in the presence of MOG protein for 3 days. IL-17A (FIG. 11I) and GM-CSF (FIG. 11J) concentrations in the culture media were analyzed by ELISA. (n=8 for PBS and SA-IL-4; n=7 for other treatment groups) FIGS. 11K-11L—Splenocytes were cultured in vitro in the presence of MOG₃₅₋₅₅ peptide for 6 hr. Cytokine expression within CD4⁺ T cells was characterized by flow cytometry. PBS and SA-IL-4: n=8, other treatment groups: n=7. The experiment was performed once. Data are mean±SEM. Statistical analyses were performed using one-way ANOVA with Tukey's test.

FIGS. 12A-12B—Disease progression with disease incidence (FIG. 12A) and body weight change (FIG. 12B) in C57BL/6 myelin oligodendrocyte glycoprotein (MOG)35-55 experimental autoimmune encephalomyelitis (EAE) mice injected every other day for 10 days from day 8 after immunization with phosphate-buffered saline (PBS) subcutaneously (s.c.), SA-IL-33 (13-39 μg, based on IL-33) subcutaneous., or SA-IL-4 (10 μg, IL-4 base) or dosed with FTY720 1 mg/kg daily by oral administration. n=6-7 per group.

FIGS. 13A-13D—Albumin fusion to IL-10 provided FcRn binding and resulted in LN accumulation. FIG. 13A—SDS-PAGE analysis for wt IL-10 and SA-IL-10. FIG. 13B—Binding analysis of SA-IL-10 to FcRn. FIG. 13C—Splenocytes (i) or single cells from the popliteal LN (ii) were incubated with SA or SA-IL-10 for 30 min on ice. Binding of each protein to immune cells was detected by co-staining with an anti-SA antibody and antibodies for specific markers of each immune cell population. FIG. 13D—Immunofluorescence images of the popliteal LN after intravenous injection of DyLight594-labeled wt IL-10 or SA-IL-10. T cells and high endothelial venules (HEVs) were respectively stained with anti-CD3 or anti-PNAd antibodies.

FIGS. 14A-14B—Albumin fusion to IL-10 provided prolonged blood circulation. FIG. 14A—wt IL-10 or SA-IL-10 (each equivalent to 35 μg of IL-10) were administered to BALB/c mice via tail vein injection. Serum was collected at the indicated time points. The serum concentration of IL-10 was measured by ELISA (mean±SEM; n=5). Plasma half-lives of IL-10 were calculated using two-phase exponential decay: MFI (t)=Ae^{-αt}+Be^{-βt}, t_{1/2, α}, fast clearance half-life; t_{1/2, β}, slow clearance half-life. Area under curve (AUC) was analyzed by GraphPad Prism. FIG. 14B—Arthritis (CAIA) was induced selectively in the right hind paw by passive immunization of anti-collagen antibodies, followed by subcutaneous injection of LPS at right hind footpad (defined as Day 3). On the day following LPS injection, DyLight800-labeled wt IL-10 or SA-IL-10 were intravenously injected in the CAIA mice. Four hours after injection, indicated organs were harvested and analyzed using an IVIS imaging system. (mean±SEM; n=4). Statistical analyses were done using a two-tailed Student's t-test. *P<0.05.

FIGS. 15A-15F—Albumin-fused IL-10 accumulated within and suppressed Th17 activation in LNs. Arthritis (CAIA) was induced by passive immunization of anti-collagen antibodies, followed by intraperitoneal injection of LPS (defined as Day 3). On the day LPS injection, wt IL-10 or SA-IL-10 were intravenously injected into the arthritic mice. IL-10 levels and Th17-relating cytokines in LNs were measured using ELISA. FIG. 15A—Comparison of IL-10 levels 4 hr after injection of each protein. FIG. 15B—Pharmacokinetics of wt IL-10 or SA-IL-10 in LNs after intravenous injection. (mean±SEM; n=4) FIG. 15C—AUC of wt IL-10 and SA-IL-10 in various LNs. FIGS. 15D and 15E—Th17-relating cytokine levels in joint-draining (popliteal) LN (FIG. 15D) and a non-draining (cervical) LN (FIG. 15E). FIG. 15F—GM-CSF levels in the popliteal LN. (mean±SEM; n=7) Statistical analyses were done using analysis of a two-tailed Student's t-test for (FIGS. 15D and 15E) or variance (ANOVA) with Tukey's test for (a and f). *P<0.05; **P<0.01; ****P<0.0001; ns; not significant.

FIGS. 16A-16B—Effect of albumin-fused IL-10 on immune cell populations in the spleen (FIG. 16A) and LNs (FIG. 16B). Arthritis (CAIA) was induced by passive immunization of anti-collagen antibodies, followed by intraperitoneal injection of LPS (defined as Day 3). On Day 3 and Day 6, PBS, wt IL-10, or SA-IL-10 were intravenously injected to mice. Single cells were extracted from the spleen and the popliteal LN on the day following the last injection, followed by flow cytometric analysis. Graphs depict the frequency of CD3⁺ T cells within live cells, CD45⁺ lymphocytes within live cells, CD11b⁺ cells within live cells, CD11c⁺ cells within CD11b cells, CD86⁺ cells within CD11c cells, granulocytic MDSC/neutrophils (Ly6G⁺Ly6C⁺ within CD11b⁺ cells), monocytic MDSC (Ly6G⁻ Ly6C⁺ within CD11b⁺ cells), macrophages (F4/80⁺ within CD11b cells), CD86⁺ cells within macrophages, and M2 macrophages (CD206⁺ F4/80⁺ within CD11b⁺ cells. (mean±SEM; n=6-7) Statistical analyses were done using analysis of variance (ANOVA) with Tukey's test except for the following graphs. For analysis of % CD11c⁺ within CD11b⁺ cells in FIG. 16A and % Ly6G⁺, Ly6C⁺ within CD11b⁺ cells in FIG. 16B, Kruskal-Wallis test followed by Dunn's multiple comparison test was employed.*P<0.05; **P<0.01; ***P<0.001; ****P<0.0001.

FIGS. 17A-17C—Albumin-fused IL-10 suppressed arthritis development more effectively than wt IL-10. FIG. 17A—Arthritis (CAIA) was induced by passive immunization with anti-collagen antibodies, followed by intraperitoneal injection of LPS. On the day of LPS injection, PBS, wt IL-10, or SA-IL-10 (equivalent to 43.5 μg of IL-10) was injected intravenously into the arthritic mice. Arthritis scores represent the mean+SEM from 7 mice. FIG. 17B—Representative H&E images of joints on day 14 in each treatment group. Scale bar, 500 m. The severity of synovial hyperplasia and bone resorption was scored 0 to 4. (mean±SEM; n=7). FIG. 17C—Effect of administration routes on therapeutic effects of SA-IL-10. Arthritis scores represent the mean+SEM from 7 mice. Statistical analyses were done using analysis of variance (ANOVA) with Tukey's test for (a and c) and a two-tailed Student's t-test for (b). **P<0.01; ***P<0.001; ****P<0.0001.

FIGS. 18A-18D—Albumin-fused IL-10 showed improved therapeutic effect on established arthritis. DBA/1J male mice were subcutaneously injected with bovine collagen/CFA emulsion in the tail base. After three weeks, bovine collagen/IFA emulsion was further injected as a boost. When arthritis scores become 2-4 (defined as Day 0), mice were intravenously injected with PBS, SA-IL-10 (each equivalent to 43.5 ag of IL-10), or with 200 μg of anti-TNF-α antibody. For the studies shown in FIGS. 18C and 18D, the same treatments were additionally injected to the mice on Day 3. In FIGS. 18A and 18C, arthritis scores represent the mean+SEM from 9-15 mice. FIGS. 18B and 18D show representative H&E histological image of joints on day 16. Scale bars, 500 m. The severity of synovial hyperplasia and bone resorption was scored 0 to 4 as described in Materials and Methods. Statistical analyses were done using a two-tailed Student's t-test. *P<0.05; **P<0.01; ***P<0.001; ns; not significant.

FIGS. 19A-19B—Albumin-fused IL-10 suppressed inflammatory responses within the paws. Arthritis (CAIA) was induced by passive immunization of anti-collagen antibodies, followed by intraperitoneal injection of LPS. On the day of LPS injection (defined as Day 3), PBS, wt IL-10 or SA-IL-10 were intravenously injected into the arthritic mice. FIG. 19A—Single cells were extracted from the hind paws on day 11, followed by flow cytometric analysis. Graphs depict the frequency of CD45⁺ cells, B cells (B220⁺ cells within CD45⁺ lymphocytes), dendritic cells (CD11c⁺ cells within CD45⁺ lymphocytes), monocytes (CD11b⁺ cells within CD45⁺ lymphocytes), granulocytic MDSC/neutrophils (Ly6G⁺Ly6C⁺CD11b⁺CD45⁺), monocytic MDSC (Ly6G⁻ Ly6C⁺CD11b⁺ CD45⁺), macrophages (F4/80⁺ CD11b⁺ CD45⁺), M2 macrophages (CD206⁺ F4/80⁺ CD11b⁺ CD45⁺), and M1 macrophages (MHC II⁺ F4/80⁺ CD11b⁺ CD45⁺). (mean±SEM; n=7) FIG. 19B—Cytokine levels in hind paws on day 11 (n=5-7). Statistical analyses were done using analysis of variance (ANOVA) with Tukey's test except for % CD11c⁺ in FIG. 19A. For analysis of % CD11c⁺ in FIG. 19A, Kruskal-Wallis test followed by Dunn's multiple comparison test was employed. *P<0.05; **P<0.01; ***P<0.001; ****P<0.0001.

FIGS. 20A-20B—Effect of albumin-fused IL-10 on T cell populations in paws and blood. Arthritis (CAIA) was induced by passive immunization of anti-collagen antibodies, followed by intraperitoneal injection of LPS (defined as Day 3). On the day of LPS injection, PBS, wt IL-10 or SA-IL-10 were intravenously injected to mice. FIG. 20A—Single cells were extracted from the hind paws on day 11, followed by flow cytometric analysis. Graphs depict the frequency of NK1.1⁺ CD3⁻ NK cells within CD45⁺ lymphocytes, CD3⁺ T cells within CD45⁺ lymphocytes, CD3⁺CD4⁺ T cells within CD45⁺ lymphocytes, Treg (Foxp3⁺CD25⁺) of CD3⁺CD4⁺ T cells, CD3⁺CD8⁺ T cells within CD45⁺ lymphocytes, effector memory T cells (CD62L⁻CD44⁺) of CD3⁺CD8⁺ T cells, central memory T cells (CD62L⁺CD44⁺) of CD3⁺ CD8⁺ T cells, PD-1⁺ cells of CD3⁺CD8⁺ T cells. FIG. 20B—Lymphocytes were extracted from blood on day 11, followed by flow cytometric analysis. Graphs depict the frequency of CD3⁺ T cells within CD45⁺ lymphocytes, CD3⁺CD4⁺ T cells within CD45⁺ lymphocytes, Treg (Foxp3⁺CD25⁺) of CD3⁺CD4⁺ T cells, CD3⁺CD8⁺ T cells within CD45⁺ lymphocytes. (mean±SEM; n=5-7) Statistical analyses were done using analysis of variance (ANOVA) with Tukey's test except for the following graphs: for analysis of % NK1.1⁺ within CD45⁺ cells, % Foxp3⁺ within CD4⁺ cells, % CD44⁺/CD62L⁻ within CD8⁺ cells, % CD44⁺/CD62L⁺ within CD8⁺ cells and % PD-1⁺ within CD8⁺ cells in (a), Kruskal-Wallis test followed by Dunn's multiple comparison test was employed. *P<0.05; **P<0.01; ***P<0.001.

FIGS. 21A-21B—Safety assessments of albumin-fused IL-10. PBS, wt IL-10 or SA-IL-10 were intravenously injected to healthy BALB/c mice. FIG. 21A—Two days after injection, white blood cell counts, red blood cell counts, platelet counts, the concentration of hemoglobin in blood and the weight of spleen were assessed. FIG. 21B—The concentrations of alanine transaminase (ALT), amylase, blood urea nitrogen (BUN), serum calcium, creatine kinase (CK), CO₂, total bilirubin (TBli) and total proteins in serum were assessed using biochemistry analyzer. (mean±SEM; n=5) Statistical analyses were done using analysis of variance (ANOVA) with Tukey's test. *P<0.05; **P<0.01.

FIGS. 22A and 22B—PBS and 100 μg anti-TNF-α were injected intraperitoneally every two days beginning on day 0 for 14 days. FTY720 (1 mg/kg body weight) was administered orally every day. SA-IL-10 (equivalent to 43.5 μg of IL-10) was injected subcutaneously on days 0 and 8. Mice were challenged subcutaneously in the front hocks on day 5 with 10 μg endotoxin-free ovalbumin, 50 μg alum, and 5 μg MPLA. Mice were bled on days 13 (a) and 19 (b), and plasma was analyzed for anti-ovalbumin total IgG titers. (mean±SEM; n=5) Statistical analyses were done using analysis of variance (ANOVA) with Tukey's test. ns; not significant.

FIG. 23 shows results from studies described in Example 3.

FIGS. 24A-24B show results from studies described in Example 4.

FIG. 25 shows results from studies described in Example 5.

FIG. 26. Percent re-epithelization of each wound measured via H&E staining.

FIG. 27. Percent re-epithelization of each wound measured via H&E staining

FIG. 28. Use of albumin fused cytokines for scleroderma therapy described in Example 7.

FIG. 29A-D. Dosing optimization for SA-IL-4. Mice were dosed subcutaneously either once or three times a week with SA-IL-4. A) Mouse weight over time. B) Expression level of CD23 on B cells isolated from peripheral blood mononuclear cells after 4 weeks of treatment. C. Total IgE levels in serum of mice isolated from mice after 4 weeks of treatment. D) Expression level of CD206 on macrophages isolated from peripheral blood mononuclear cells after 1 week of treatment.

FIG. 30A-B. Wild type IL-33 causes severe toxicity in EAE-bearing mice. Mice were induced with EAE on day 0 and treated with 26 μg of wild type (WT) IL-33, equimolar SA-IL-33, or PBS subcutaneously every other day beginning on day 8. A) Survival curve of mice treated with wild type IL-33, SA-IL-33, or PBS. B) EAE clinical score of mice. Surviving mice receiving WT-IL-33 were removed from clinical score data after day 11 due to the insufficient number of mice to power the study.

FIG. 31A-D. Three doses of SA-IL-33 is sufficient to prevent from EAE while mitigating toxicity. Healthy C57BL/6 mice received various doses of wild type (WT) or equimolar SA-IL-33 subcutaneously every other day (A-B). Mice were bled and IgE levels were measured in the serum at A) day 5 and B) day 9. Mice were induced with EAE at day 0 and treated every other day subcutaneously with 26 μg SA-IL-33 at day 8 for either 3 doses or 8 doses. C) EAE clinical score over time. D) Clinical score at day 20.

FIG. 32. Representative SA IL-33 size exclusion chromatography plot following affinity and size exclusion chromatography on AKTA pure.

FIG. 33. SA IL-33 SDS page (S) under non-reducing conditions with Coomassie blue staining and anti-histidine Western blot (W).

FIG. 34. Binding Affinity of Fc-ST2 to SA IL-33 on NTA chip measured by SPR.

FIG. 35A-B. SA Fusion to IL-33 prolongs its concentration in blood plasma after subcutaneous injection. (A) SA IL-33 in vivo pharmacokinetics study overview. (B) Comparison of WT vs. SA IL-33 plasma pharmacokinetics.

FIG. 36A-G. SA IL-33 treatment prevents the onset of MOG-induced EAE in the acute phase. (A) SA IL-33 dose escalation study in prophylactic EAE overview. Mice were treated with 13 ug, 26 ug, or 39 ug (wt IL-33 molar equivalent dose) by subcutaneous injection on days 8, 10, 12 and 14 post EAE immunization. (B) Treatment with 26 ug and 39 ug SA IL-33 per injection prevents the onset of EAE. EAE clinical scores from day 7 to 15 and clinical scores on day 15. (C) EAE mice treated with 26 ug SA IL-33 maintain body weight whereas PBS-treated mice lose weight after day 10 post-immunization. (D) SA IL-33 treatment increases the frequency of ST2+ FoxP3⁺CD25⁺ regulatory T cells in the spinal cord draining lymph nodes and spleen in acute phase MOG-induced EAE mice. (E) SA IL-33 treatment increases the frequency of Th2 CD4⁺ T cells in the spinal cord draining lymph nodes & spleen and M2 macrophages in spleen of acute phase EAE mice. (F) SA IL-33 treatment increases the frequency of group 2 innate lymphoid cells in the spleen of acute phase MOG-induced EAE mice. (G) SA IL-33 treatment reduces lymphocyte infiltration and cytokine production in the spinal cord of acute phase MOG-induced EAE mice.

FIG. 37A-F. SA IL-33 treatment in the chronic phase of EAE reduces clinical score, increases body weight, and reduces immune-cell infiltration. (A) SA IL-33 treatment in chronic EAE overview. Mice were treated with 26 ug (wt IL-33 molar equivalent dose) by subcutaneous injection on days 20, 22, 24, 26, 28, 30, 32, and 34 post EAE immunization. (B) Treatment with 26 ug SA IL-33 per injection in the chronic phase reduces EAE clinical score. EAE clinical scores from day 7 to 34 and clinical scores on day 34. (C) SA IL-33 treatment in the chronic phase of EAE induces weight gain. (D) SA IL-33 treatment in the chronic phase of EAE increases the frequency of ST2+ FoxP3+ CD25+ regulatory T cells in the spinal cord draining lymph nodes in the chronic phase of MOG-induced EAE mice. (E) SA IL-33 treatment reduces lymphocyte infiltration and cytokine production in the spinal cord of chronic phase MOG-induced EAE mice. (F) SA IL-33 treated EAE mouse splenocytes restimulated in vitro with MOG protein or peptide showed reduced TNFa, IL-17A and IL-17F production compared to PBS treated mice.

FIG. 38A-C. SA IL-33 treatment prevents the onset of MOG-induced EAE in the acute phase for at least ten days after cessation of treatment. (A) SA IL-33 dose number study in prophylactic EAE overview. Mice were treated with 26 ug (wt IL-33 molar equivalent dose) by subcutaneous injection on days 8, 10, 12 post EAE immunization or days 8, 10, 12, 14, 16, 18, 20, 22 post EAE immunization. (B) Treatment with three doses of 26 ug SA IL-33 per injection in the acute phase prevents the onset of EAE for at least ten days after cessation of treatment. EAE clinical scores from day 7 to day 20 and clinical scores on day 20. (C) EAE mice treated with three doses of 26 ug SA IL-33 per injection in the acute phase maintain body weight for at least ten days after cessation of treatment.

FIG. 39A-B. EAE mice experience greatly reduced toxicity compared to naïve mice. C57BL/6 mice were induced with EAE as previously described. On day 21, EAE mice were treated with PBS (EAE) or 10 μg SA-IL-4 (EAE+SA-IL-4) subcutaneously every other day starting on day 0. Simultaneously, aged-matched C57BL/6 mice were treated with PBS (PBS) or 10 μg SA-IL-4 (SA-IL-4) every other day starting on day 0. A) Survival curve of mice to day 15 after 1^{st} injection. B) Mouse weights to day 15 after 1^{st} injection.

FIG. 40A-B. Non-obese diabetic (NOD) mice experience reduced toxicity compared to naïve mice. NOD mice were treated 3 times per week subcutaneously with 10 μg SA-IL-4 (NOD) beginning on day 0. Simultaneously, aged-matched C57BL/6 mice were treated with PBS (PBS) or 10 μg SA-IL-4 (SA-IL-4) every other day starting on day 0. A) Survival curve of mice to day 45 after 1^{st} injection. B) Mouse weights to day 45 after 1^{st} injection.

FIG. 41A-B. Design of SA IL-35 fusion protein. (A) Diagram of SAIL-35 plasmid design. (B) Purified fraction of SA IL-35 after affinity and size exclusion chromatography. Image of ladder (left) and SDS page SA IL-35 (right).

FIG. 42. Albumin-fused IL-35 suppressed arthritis development. Arthritis (CAIA) was induced by passive immunization with anti-collagen antibodies, followed by intraperitoneal injection of LPS on day 3. On the day of LPS injection, PBS, SA-IL-10, SA-IL-27, SA-IL-35, or SA-IL-37 was injected subcutaneously into the arthritic mice. Mice were sacrificed on day 11. Arthritis scores represent the mean+SEM from 7 mice. Arthritis scores represent the mean+SEM from 7 mice.

### DETAILED DESCRIPTION

Aspects of this disclosure fulfill certain needs in the art by providing compositions comprising anti-inflammatory cytokines, in some cases linked to an albumin protein, and methods for treatment of autoimmune and inflammatory conditions and for promoting wound healing. The present disclosure is based, at least in part, on the surprising discovery that administration of an anti-inflammatory cytokine linked to an albumin protein is effective in treating various autoimmune or inflammatory conditions, as well as in promoting wound healing. Also described herein are methods for targeting an anti-inflammatory cytokine to a lymph node of a subject by linking the cytokine to an albumin protein.

### I. PROTEINS

Aspects of the disclosure are directed to various proteins and methods of use. As used herein, a “protein” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, or more contiguous amino acids or nucleic acids, or any range derivable therein, of any of SEQ ID NOs:1-51.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, or 304, 305, or 306 (or any derivable range therein) of any of SEQ ID NOs:1-51.

In some aspects, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, or 306 contiguous amino acids of any of SEQ ID NOs:1-51 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-51.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, or 305 of any of SEQ ID NOS1-51 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, or 306 (or any derivable range therein) contiguous amino acids of any of SEQ ID NOS:1-51.

The polypeptides of the disclosure may comprise a substitution at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS:1-51 (or any derivable range therein) and may be a substitution with alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

A. Anti-Inflammatory Polypeptides

Aspects of the present disclosure are directed to comparisons comprising anti-inflammatory polypeptides and their methods of use. In some aspects, disclosed are anti-inflammatory polypeptides useful in methods for treatment of an autoimmune or inflammatory condition. In some aspects, disclosed are anti-inflammatory polypeptides useful in methods for promoting wound healing. “Anti-inflammatory polypeptides” describe any polypeptide capable of reducing, inhibiting, preventing, or eliminating an inflammatory response in a subject. In some aspects, an anti-inflammatory polypeptide is capable of decreasing a number and/or function of Th17 cells in a subject. Examples of anti-inflammatory polypeptides include anti-inflammatory cytokines, polypeptides comprising an anti-inflammatory cytokine and an albumin protein, and polypeptides capable of binding to and inhibiting activity of an inflammatory cytokine. In some aspects, an anti-inflammatory polypeptide of the present disclosure comprises an anti-inflammatory cytokine. In some aspects, an anti-inflammatory polypeptide of the present disclosure is an anti-inflammatory cytokine. In some aspects, an anti-inflammatory cytokine is operatively linked (e.g., covalently linked or non-covalently linked) to one or more additional polypeptides. In some aspects, an anti-inflammatory cytokine is operatively linked to an albumin protein. In some aspects, an anti-inflammatory polypeptide of the disclosure is an anti-inflammatory cytokine covalently linked to an albumin protein. In some aspects, an anti-inflammatory polypeptide of the disclosure is an anti-inflammatory cytokine covalently linked to an albumin binding protein. An anti-inflammatory cytokine may be linked to an additional polypeptide (e.g., an albumin protein, an albumin binding protein) via one or more linkers. An additional polypeptide (e.g., an albumin protein, an albumin binding protein) may be linked at an N-terminus of an anti-inflammatory cytokine. An additional polypeptide (e.g., an albumin protein, an albumin binding protein) may be linked at a C-terminus of an anti-inflammatory cytokine. It is also contemplated that an anti-inflammatory cytokine may be conjugated to albumin, such as through chemical conjugation. In some aspects, the linker is a non-amino acid linker, such as an azide or thiol linker, which are widely available.

Certain, non-limiting examples of anti-inflammatory polypeptides contemplated herein are provided in Table 1.

TABLE 1

| Anti-inflammatory | | SEQ ID |
|---|---|---|
| polypeptide | Sequence | NO: |
| Human IL-4 linked to | DAHKSEVAHRFKDLGEENFKALVLIAFAQYLQQC | 5 |
| human albumin via | PFEDHVKLVNEVTEFAKTCVADESAENCDKSLHT | |
| (GGGS)2 linker | LFGDKLCTVATLRETYGEMADCCAKQEPERNECF | |
| | LQHKDDNPNLPRLVRPEVDVMCTAFHDNEETFLK | |
| | KYLYEIARRHPYFYAPELLFFAKRYKAAFTECCQA | |
| | ADKAACLLPKLDELRDEGKASSAKQRLKCASLQ | |
| | KFGERAFKAWAVARLSQRFPKAEFAEVSKLVTDLT | |
| | KVHTECCHGDLLECADDRADLAKYICENQDSISS | |
| | KLKECCEKPLLEKSHCIAEVENDEMPADLPSLAA | |
| | DFVESKDVCKNYAEAKDVFLGMFLYEYARRHPD | |
| | YSVVLLLRLAKTYETTLEKCCAAADPHECYAKVF | |
| | DEFKPLVEEPQNLIKQNCELFEQLGEYKFQNALLV | |
| | RYTKKVPQVSTPTLVEVSRNLGKVGSKCCKHPEA | |
| | KRMPCAEDYLSVVLNQLCVLHEKTPVSDRVTKC | |
| | CTESLVNRRPCFSALEVDETYVPKEFNAETFTFHA | |
| | DICTLSEKERQIKKQTALVELVKHKPKATKEQLKA | |
| | VMDDFAAFVEKCCKADDKETCFAEEGKKLVAAS | |
| | QAALGLGGGSGGGSHKCDITLQEIIKTLNSLTEQK | |
| | TLCTELTVTDIFAASKNTTEKETFCRAATVLRQFYS | |
| | HHEKDTRCLGATAQQFHRHKQLIRFLKRLDRNLW | |
| | GLAGLNSCPVKEANQSTLENFLERLKTIMREKYS | |
| | KCSS | |
| Mouse IL-4 linked to | EAHKSEIAHRYNDLGEQHFKGLVLIAFSQYLQKCS | 6 |
| mouse albumin via | YDEHAKLVQEVTDFAKTCVADESAANCDKSLHTL | |
| (GGGS)2 linker | FGDKLCAIPNLRENYGELADCCTKQEPERNECFL | |
| | QHKDDNPSLPPFERPEAEAMCTSFKENPTTFMGH | |
| | YLHEVARRHPYFYAPELLYYAEQYNEILTQCCAEA | |
| | DKESCLTPKLDGVKEKALVSSVRQRMKCSSMQKF | |
| | GERAFKAWAVARLSQTFPNADFAEITKLATDLTKV | |
| | NKECCHGDLLECADDRAELAKYMCENQATISSKL | |
| | QTCCDKPLLKKAHCLSEVEHDTMPADLPAIAADF | |
| | VEDQEVCKNYAEAKDVFLGTFLYEYSRRHPDYSV | |
| | SLLLRLAKKYEATLEKCCAEANPPACYGTVLAEF | |
| | QPLVEEPKNLVKTNCDLYEKLGEYGFQNAILVRYT | |
| | QKAPQVSTPTLVEAARNLGRVGTKCCTLPEDQRL | |
| | PCVEDYLSAILNRVCLLHEKTPVSEHVTKCCSGSL | |
| | VERRPCFSALTVDETYVPKEFKAETFTFHSDICTLP | |
| | EKEKQIKKQTALAELVKHKPKATAEQLKTVMDDF | |
| | AQFLDTCCKAADKDTCFSTEGPNLVTRCKDALAG | |
| | GGGSGGGSMHIHGCDKNHLREIIGILNEVTGEGTP | |
| | CTEMDVPNVLTATKNTTESELVCRASKVLRIFYLK | |
| | HGKTPCLKKNSSVLMELQRLFRAFRCLDSSISCTM | |
| | NESKSTSLKDFLESLKSIMQMDYS | |
| Human IL-33 linked to | DAHKSEVAHRFKDLGEENFKALVLIAFAQYLQQC | 9 |
| human albumin via | PFEDHVKLVNEVTEFAKTCVADESAENCDKSLHT | |
| (GGGS)2 linker | LFGDKLCTVATLRETYGEMADCCAKQEPERNECF | |
| | LQHKDDNPNLPRLVRPEVDVMCTAFHDNEETFLK | |
| | KYLYEIARRHPYFYAPELLFFAKRYKAAFTECCQA | |
| | ADKAACLLPKLDELRDEGKASSAKQRLKCASLQ | |
| | KFGERAFKAWAVARLSQRFPKAEFAEVSKLVTDLT | |
| | KVHTECCHGDLLECADDRADLAKYICENQDSISS | |
| | KLKECCEKPLLEKSHCIAEVENDEMPADLPSLAA | |
| | DFVESKDVCKNYAEAKDVFLGMFLYEYARRHPD | |
| | YSVVLLLRLAKTYETTLEKCCAAADPHECYAKVF | |
| | DEFKPLVEEPQNLIKQNCELFEQLGEYKFQNALLV | |
| | RYTKKVPQVSTPTLVEVSRNLGKVGSKCCKHPEA | |
| | KRMPCAEDYLSVVLNQLCVLHEKTPVSDRVTKC | |
| | CTESLVNRRPCFSALEVDETYVPKEFNAETFTFHA | |
| | DICTLSEKERQIKKQTALVELVKHKPKATKEQLKA | |
| | VMDDFAAFVEKCCKADDKETCFAEEGKKLVAAS | |
| | QAALGLGGGSGGGSAFGISGVQKYTRALHDSSIT | |
| | GISPITEYLASLSTYNDQSITFALEDESYEIYVEDLK | |
| | KDEKKDKVLLSYYESQHPSNESGDGVDGKMLMV | |
| | TLSPTKDFWLHANNKEHSVELHKCEKPLPDQAFF | |
| | VLHNMHSNCVSFECKTDPGVFIGVKDNHLALIKV | |
| | DSSENLCTENILFKLSET | |
| Mouse IL-33 linked to | EAHKSEIAHRYNDLGEQHFKGLVLIAFSQYLQKCS | 10 |
| mouse albumin via | YDEHAKLVQEVTDFAKTCVADESAANCDKSLHTL | |
| (GGGS)2 linker | FGDKLCAIPNLRENYGELADCCTKQEPERNECFL | |
| | QHKDDNPSLPPFERPEAEAMCTSFKENPTTFMGH | |
| | YLHEVARRHPYFYAPELLYYAEQYNEILTQCCAEA | |
| | DKESCLTPKLDGVKEKALVSSVRQRMKCSSMQKF | |
| | GERAFKAWAVARLSQTFPNADFAEITKLATDLTKV | |
| | NKECCHGDLLECADDRAELAKYMCENQATISSKL | |
| | QTCCDKPLLKKAHCLSEVEHDTMPADLPAIAADF | |
| | VEDQEVCKNYAEAKDVFLGTFLYEYSRRHPDYSV | |
| | SLLLRLAKKYEATLEKCCAEANPPACYGTVLAEF | |
| | QPLVEEPKNLVKTNCDLYEKLGEYGFQNAILVRYT | |
| | QKAPQVSTPTLVEAARNLGRVGTKCCTLPEDQRL | |
| | PCVEDYLSAILNRVCLLHEKTPVSEHVTKCCSGSL | |
| | VERRPCFSALTVDETYVPKEFKAETFTFHSDICTLP | |
| | EKEKQIKKQTALAELVKHKPKATAEQLKTVMDDF | |
| | AQFLDTCCKAADKDTCFSTEGPNLVTRCKDALAG | |
| | GGSGGGSAASVDTLSIQGTSLLTQSPASLSTYNDQ | |
| | SVSFVLENGCYVINVDDSGKDQEQDQVLLRYYES | |
| | PCPASQSGDGVDGKKLMVNMSPIKDTDIWLHAN | |
| | DKDYSVELQRGDVSPPEQAFFVLHKKSSDFVSFE | |
| | CKNLPGTYIGVKDNQLALVEEKDESCNNIMFKLS | |
| | KI | |
| Mouse IgK signal | METDTLLLWVLLLWVPGSTGDGEAHKSEIAHRYN | 52 |
| peptide-Mouse Serum | DLGEQHFKGLVLIAFSQYLQKCSYDEHAKLVQEV | |
| Albumin-Mouse IL-33 | TDFAKTCVADESAANCDKSLHTLFGDKLCAIPNL | |
| | RENYGELADCCTKQEPERNECFLQHKDDNPSLPP | |
| | FERPEAEAMCTSFKENPTTFMGHYLHEVARRHPY | |
| | FYAPELLYYAEQYNEILTQCCAEADKESCLTPKLD | |
| | GVKEKALVSSVRQRMKCSSMQKFGERAFKAWAV | |
| | ARLSQTFPNADFAEITKLATDLTKVNKECCHGDLL | |
| | ECADDRAELAKYMCENQATISSKLQTCCDKPLLK | |
| | KAHCLSEVEHDTMPADLPAIAADFVEDQEVCKNY | |
| | AEAKDVFLGTFLYEYSRRHPDYSVSLLLRLAKKY | |
| | EATLEKCCAEANPPACYGTVLAEFQPLVEEPKNLV | |
| | KTNCDLYEKLGEYGFQNAILVRYTQKAPQVSTPT | |
| | LVEAARNLGRVGTKCCTLPEDQRLPCVEDYLSAIL | |
| | NRVCLLHEKTPVSEHVTKCCSGSLVERRPCFSALT | |
| | VDETYVPKEFKAETFTFHSDICTLPEKEKQIKKQT | |
| | ALAELVKHKPKATAEQLKTVMDDFAQFLDTCCK | |
| | AADKDTCFSTEGPNLVTRCKDALAGGGGSGGGSS | |
| | IQGTSLLTQSPASLSTYNDQSVSFVLENGCYVINV | |
| | DDSGKDQEQDQVLLRYYESPCPASQSGDGVDGK | |
| | KLMVNMSPIKDTDIWLHANDKDYSVELQRGDVS | |
| | PPEQAFFVLHKKSSDFVSFECKNLPGTYIGVKDNQ | |
| | LALVEEKDESCNNIMFKLSKI | |
| Human IL-10 linked to | DAHKSEVAHRFKDLGEENFKALVLIAFAQYLQQC | 13 |
| human albumin via | PFEDHVKLVNEVTEFAKTCVADESAENCDKSLHT | |
| (GGGS)2 linker | LFGDKLCTVATLRETYGEMADCCAKQEPERNECF | |
| | LQHKDDNPNLPRLVRPEVDVMCTAFHDNEETFLK | |
| | KYLYEIARRHPYFYAPELLFFAKRYKAAFTECCQA | |
| | ADKAACLLPKLDELRDEGKASSAKQRLKCASLQ | |
| | KFGERAFKAWAVARLSQRFPKAEFAEVSKLVTDLT | |
| | KVHTECCHGDLLECADDRADLAKYICENQDSISS | |
| | KLKECCEKPLLEKSHCIAEVENDEMPADLPSLAA | |
| | DFVESKDVCKNYAEAKDVFLGMFLYEYARRHPD | |
| | YSVVLLLRLAKTYETTLEKCCAAADPHECYAKVF | |
| | DEFKPLVEEPQNLIKQNCELFEQLGEYKFQNALLV | |
| | RYTKKVPQVSTPTLVEVSRNLGKVGSKCCKHPEA | |
| | KRMPCAEDYLSVVLNQLCVLHEKTPVSDRVTKC | |
| | CTESLVNRRPCFSALEVDETYVPKEFNAETFTFHA | |
| | DICTLSEKERQIKKQTALVELVKHKPKATKEQLKA | |
| | VMDDFAAFVEKCCKADDKETCFAEEGKKLVAAS | |
| | QAALGLGGGSGGGSSPGQGTQSENSCTHFPGNLP | |
| | NMLRDLRDAFSRVKTFFQMKDQLDNLLLKESLLE | |
| | DFKGYLGCQALSEMIQFYLEEVMPQAENQDPDIK | |
| | AHVNSLGENLKTLRLRLRRCHRFLPCENKSKAVE | |
| | QVKNAFNKLQEKGIYKAMSEFDIFINYIEAYMTM | |
| | KIRN | |
| Mouse IL-10 linked to | EAHKSEIAHRYNDLGEQHFKGLVLIAFSQYLQKCS | 14 |
| mouse albumin via | YDEHAKLVQEVTDFAKTCVADESAANCDKSLHTL | |
| (GGGS)2 linker | FGDKLCAIPNLRENYGELADCCTKQEPERNECFL | |
| | QHKDDNPSLPPFERPEAEAMCTSFKENPTTFMGH | |
| | YLHEVARRHPYFYAPELLYYAEQYNEILTQCCAEA | |
| | DKESCLTPKLDGVKEKALVSSVRQRMKCSSMQKF | |
| | GERAFKAWAVARLSQTFPNADFAEITKLATDLTKV | |
| | NKECCHGDLLECADDRAELAKYMCENQATISSKL | |
| | QTCCDKPLLKKAHCLSEVEHDTMPADLPAIAADF | |
| | VEDQEVCKNYAEAKDVFLGTFLYEYSRRHPDYSV | |
| | SLLLRLAKKYEATLEKCCAEANPPACYGTVLAEF | |
| | QPLVEEPKNLVKTNCDLYEKLGEYGFQNAILVRYT | |
| | QKAPQVSTPTLVEAARNLGRVGTKCCTLPEDQRL | |
| | PCVEDYLSAILNRVCLLHEKTPVSEHVTKCCSGSL | |
| | VERRPCFSALTVDETYVPKEFKAETFTFHSDICTLP | |
| | EKEKQIKKQTALAELVKHKPKATAEQLKTVMDDF | |
| | AQFLDTCCKAADKDTCFSTEGPNLVTRCKDALAG | |
| | GGSGGGSSRGQYSREDNNCTHFPVGQSHMLLELR | |
| | TAFSQVKTFFQTKDQLDNILLTDSLMQDFKGYLG | |
| | CQALSEMIQFYLVEVMPQAEKHGPEIKEHLNSLG | |
| | EKLKTLRMRLRRCHRFLPCENKSKAVEQVKSDEN | |
| | KLQDQGVYKAMNEFDIFINCIEAYMMIKMKS | |
| Human IL-35 linked to | RKGPPAALTLPRVQCRASRYPIAVDCSWTLPPAPN | 53 |
| human albumin via | STSPVSFIATYRLGMAARGHSWPCLQQTPTSTSCT | |
| (GGGS)2 linker | ITDVQLFSMAPYVLNVTAVHPWGSSSSFVPFITEHI | |
| | IKPDPPEGVRLSPLAERQLQVQWEPPGSWPFPEIFS | |
| | LKYWIRYKRQGAARFHRVGPIEATSFILRAVRPRA | |
| | RYYVQVAAQDLTDYGELSDWSLPATATMSLGKGG | |
| | GSGGGSGGGSGGGSRNLPVATPDPGMFPCLHHSQ | |
| | NLLRAVSNMLQKARQTLEFYPCTSEEIDHEDITKD | |
| | KTSTVEACLPLELTKNESCLNSRETSFITNGSCLAS | |
| | RKTSFMMALCLSSIYEDLKMYQVEFKTMNAKLL | |
| | MDPKRQIFLDQNMLAVIDELMQALNFNSETVPQK | |
| | SSLEEPDFYKTKIKLCILLHAFRIRAVTIDRVMSYL | |
| | NASGGGSGGGSGGGSGGGSGGGSDAHKSEVAHR | |
| | FKDLGEENFKALVLIAFAQYLQQCPFEDHVKLVN | |
| | EVTEFAKTCVADESAENCDKSLHTLFGDKLCTVAT | |
| | LRETYGEMADCCAKQEPERNECFLQHKDDNPNL | |
| | PRLVRPEVDVMCTAFHDNEETFLKKYLYEIARRHP | |
| | YFYAPELLFFAKRYKAAFTECCQAADKAACLLPK | |
| | LDELRDEGKASSAKQRLKCASLQKFGERAFKAW | |
| | AVARLSQRFPKAEFAEVSKLVTDLTKVHTECCHGD | |
| | LLECADDRADLAKYICENQDSISSKLKECCEKPLL | |
| | EKSHCIAEVENDEMPADLPSLAADFVESKDVCKN | |
| | YAEAKDVFLGMFLYEYARRHPDYSVVLLLRLAKT | |
| | YETTLEKCCAAADPHECYAKVFDEFKPLVEEPQN | |
| | LIKQNCELFEQLGEYKFQNALLVRYTKKVPQVSTP | |
| | TLVEVSRNLGKVGSKCCKHPEAKRMPCAEDYLS | |
| | VVLNQLCVLHEKTPVSDRVTKCCTESLVNRRPCFS | |
| | ALEVDETYVPKEFNAETFTFHADICTLSEKERQIK | |
| | KQTALVELVKHKPKATKEQLKAVMDDFAAFVEKC | |
| | CKADDKETCFAEEGKKLVAASQAALGL | |
| Human IL-37 linked to | DAHKSEVAHRFKDLGEENFKALVLIAFAQYLQQC | 54 |
| human albumin via | PFEDHVKLVNEVTEFAKTCVADESAENCDKSLHT | |
| (GGGS)2 linker | LFGDKLCTVATLRETYGEMADCCAKQEPERNECF | |
| | LQHKDDNPNLPRLVRPEVDVMCTAFHDNEETFLK | |
| | KYLYEIARRHPYFYAPELLFFAKRYKAAFTECCQA | |
| | ADKAACLLPKLDELRDEGKASSAKQRLKCASLQ | |
| | KFGERAFKAWAVARLSQRFPKAEFAEVSKLVTDLT | |
| | KVHTECCHGDLLECADDRADLAKYICENQDSISS | |
| | KLKECCEKPLLEKSHCIAEVENDEMPADLPSLAA | |
| | DFVESKDVCKNYAEAKDVFLGMFLYEYARRHPD | |
| | YSVVLLLRLAKTYETTLEKCCAAADPHECYAKVF | |
| | DEFKPLVEEPQNLIKQNCELFEQLGEYKFQNALLV | |
| | RYTKKVPQVSTPTLVEVSRNLGKVGSKCCKHPEA | |
| | KRMPCAEDYLSVVLNQLCVLHEKTPVSDRVTKC | |
| | CTESLVNRRPCFSALEVDETYVPKEFNAETFTFHA | |
| | DICTLSEKERQIKKQTALVELVKHKPKATKEQLKA | |
| | VMDDFAAFVEKCCKADDKETCFAEEGKKLVAAS | |
| | QAALGLGGGSGGGSGGGSGGGSGGGSVHTSPKV | |
| | KNLNPKKFSIHDQDHKVLVLDSGNLIAVPDKNYIR | |
| | PEIFFALASSLSSASAEKGSPILLGVSKGEFCLYCD | |
| | KDKGQSHPSLQLKKEKLMKLAAQKESARRPFIFY | |
| | RAQVGSWNMLESAAHPGWFICTSCNCNEPVGVT | |
| | DKFENRKHIEFSFQPVCKAEMSPSEVSD | |

B. Anti-Inflammatory Cytokines

Aspects of the present disclosure comprise anti-inflammatory cytokines. An “anti-inflammatory cytokine” describes a cytokine capable of controlling, regulating, or inhibiting an inflammatory (or “proinflammatory”) response. An anti-inflammatory cytokine of the disclosure may be from any species. An anti-inflammatory cytokine may be selected for the disclosed methods based on the desired use and outcome; for example a mouse anti-inflammatory cytokine may be selected for administration to a mouse subject while a human anti-inflammatory cytokine may be selected for administration to a human subject. In some cases, a human anti-inflammatory cytokine may be selected for administration to a mouse subject, for example where the human anti-inflammatory cytokine is capable of having an anti-inflammatory effect in the mouse.

Certain, non-limiting examples of anti-inflammatory cytokines contemplated herein are provided in Table 2.

TABLE 2

| Anti- | | |
|---|---|---|
| inflammatory | | SEQ ID |
| cytokine | Sequence | NO: |
| Human IL-4 | HKCDITLQEIIKTLNSLTEQKTLCTELTVTDIFAASKNTTEKE | 3 |
| | TFCRAATVLRQFYSHHEKDTRCLGATAQQFHRHKQLIRFLK | |
| | RLDRNLWGLAGLNSCPVKEANQSTLENFLERLKTIMREKY | |
| | SKCSS | |
| Mouse IL-4 | HIHGCDKNHLREIIGILNEVTGEGTPCTEMDVPNVLTATKNT | 4 |
| | TESELVCRASKVLRIFYLKHGKTPCLKKNSSVLMELQRLFR | |
| | AFRCLDSSISCTMNESKSTSLKDFLESLKSIMQMDYS | |
| Human IL-33 | AFGISGVQKYTRALHDSSITGISPITEYLASLSTYNDQSITFA | 7 |
| | LEDESYEIYVEDLKKDEKKDKVLLSYYESQHPSNESGDGV | |
| | DGKMLMVTLSPTKDFWLHANNKEHSVELHKCEKPLPDQA | |
| | FFVLHNMHSNCVSFECKTDPGVFIGVKDNHLALIKVDSSEN | |
| | LCTENILFKLSET | |
| Mouse IL-33 | AASVDTLSIQGTSLLTQSPASLSTYNDQSVSFVLENGCYVIN | 8 |
| | VDDSGKDQEQDQVLLRYYESPCPASQSGDGVDGKKLMVN | |
| | MSPIKDTDIWLHANDKDYSVELQRGDVSPPEQAFFVLHKK | |
| | SSDFVSFECKNLPGTYIGVKDNQLALVEEKDESCNNIMFKL | |
| | SKI | |
| Human IL-10 | SPGQGTQSENSCTHFPGNLPNMLRDLRDAFSRVKTFFQMK | 11 |
| | DQLDNLLLKESLLEDFKGYLGCQALSEMIQFYLEEVMPQA | |
| | ENQDPDIKAHVNSLGENLKTLRLRLRRCHRFLPCENKSKAV | |
| | EQVKNAFNKLQEKGIYKAMSEFDIFINYIEAYMTMKIRN | |
| Mouse IL-10 | SRGQYSREDNNCTHFPVGQSHMLLELRTAFSQVKTFFQTK | 12 |
| | DQLDNILLTDSLMQDFKGYLGCQALSEMIQFYLVEVMPQA | |
| | EKHGPEIKEHLNSLGEKLKTLRMRLRRCHRFLPCENKSKAV | |
| | EQVKSDFNKLQDQGVYKAMNEFDIFINCIEAYMMIKMKS | |
| Human IL-1ra | RPSGRKSSKMQAFRIWDVNQKTFYLRNNQLVAGYLQGPNV | 15 |
| | NLEEKIDVVPIEPHALFLGIHGGKMCLSCVKSGDETRLQLE | |
| | AVNITDLSENRKQDKRFAFIRSDSGPTTSFESAACPGWFLCT | |
| | AMEADQPVSLTNMPDEGVMVTKFYFQEDE | |
| Mouse IL-1ra | RPSGKRPCKMQAFRIWDTNQKTFYLRNNQLIAGYLQGPNI | 16 |
| | KLEEKIDMVPIDLHSVFLGIHGGKLCLSCAKSGDDIKLQLEE | |
| | VNITDLSKNKEEDKRFTFIRSEKGPTTSFESAACPGWFLCTT | |
| | LEADRPVSLTNTPEEPLIVTKFYFQEDQ | |
| Human IL-5 | IPTEIPTSALVKETLALLSTHRTLLIANETLRIPVPVHKNHQL | 17 |
| | CTEEIFQGIGTLESQTVQGGTVERLFKNLSLIKKYIDGQKKK | |
| | CGEERRRVNQFLDYLQEFLGVMNTEWIIES | |
| Mouse IL-5 | EIPMSTVVKETLTQLSAHRALLTSNETMRLPVPTHKNHQLC | 18 |
| | IGEIFQGLDILKNQTVRGGTVEMLFQNLSLIKKYIDRQKEKC | |
| | GEERRRTRQFLDYLQEFLGVMSTEWAMEG | |
| Human IL-11 | PGPPPGPPRVSPDPRAELDSTVLLTRSLLADTRQLAAQLRDK | 19 |
| | FPADGDHNLDSLPTLAMSAGALGALQLPGVLTRLRADLLS | |
| | YLRHVQWLRRAGGSSLKTLEPELGTLQARLDRLLRRLQLL | |
| | MSRLALPQPPPDPPAPPLAPPSSAWGGIRAAHAILGGLHLTL | |
| | DWAVRGLLLLKTRL | |
| Mouse IL-11 | MPGPPAGSPRVSSDPRADLDSAVLLTRSLLADTRQLAAQMR | 20 |
| | DKFPADGDHSLDSLPTLAMSAGTLGSLQLPGVLTRLRVDL | |
| | MSYLRHVQWLRRAGGPSLKTLEPELGALQARLERLLRRLQ | |
| | LLMSRLALPQAAPDQPVIPLGPPASAWGSIRAAHAILGGLH | |
| | LTLDWAVRGLLLLKTRL | |
| Human IL-23 | RAVPGGSSPAWTQCQQLSQKLCTLAWSAHPLVGHMDLREE | 21 |
| p19 | GDEETTNDVPHIQCGDGCDPQGLRDNSQFCLQRIHQGLIFY | |
| | EKLLGSDIFTGEPSLLPDSPVGQLHASLLGLSQLLQPEGHH | |
| | WETQQIPSLSPSQPWQRLLLRFKILRSLQAFVAVAARVFAHG | |
| | AATLSP | |
| Human IL-23 | IWELKKDVYVVELDWYPDAPGEMVVLTCDTPEEDGITWTL | 22 |
| p40 | DQSSEVLGSGKTLTIQVKEFGDAGQYTCHKGGEVLSHSLLL | |
| | LHKKEDGIWSTDILKDQKEPKNKTFLRCEAKNYSGRFTCW | |
| | WLTTISTDLTFSVKSSRGSSDPQGVTCGAATLSAERVRGDN | |
| | KEYEYSVECQEDSACPAAEESLPIEVMVDAVHKLKYENYTS | |
| | SFFIRDIIKPDPPKNLQLKPLKNSRQVEVSWEYPDTWSTPHS | |
| | YFSLTFCVQVQGKSKREKKDRVFTDKTSATVICRKNASISV | |
| | RAQDRYYSSSWSEWASVPCS | |
| Human IL-27 | FPRPPGRPQLSLQELRREFTVSLHLARKLLSEVRGQAHRFA | 23 |
| p28 | ESHLPGVNLYLLPLGEQLPDVSLTFQAWRRLSDPERLCFIST | |
| | TLQPFHALLGGLGTQGRWTNMERMQLWAMRLDLRDLQR | |
| | HLRFQVLAAGFNLPEEEEEEEEEEEEERKGLLPGALGSALQ | |
| | GPAQVSWPQLLSTYRLLHSLELVLSRAVRELLLLSKAGHSV | |
| | WPLGFPTLSPQP | |
| Human IL-27 | RKGPPAALTLPRVQCRASRYPIAVDCSWTLPPAPNSTSPVSFI | 24 |
| EBI3 | ATYRLGMAARGHSWPCLQQTPTSTSCTITDVQLFSMAPYV | |
| | LNVTAVHPWGSSSSFVPFITEHIIKPDPPEGVRLSPLAERQLQ | |
| | VQWEPPGSWPFPEIFSLKYWIRYKRQGAARFHRVGPIEATSF | |
| | ILRAVRPRARYYVQVAAQDLTDYGELSDWSLPATATMSLGK | |
| Mouse IL-27 | FPTDPLSLQELRREFTVSLYLARKLLSEVQGYVHSFAESRLP | 25 |
| p28 | GVNLDLLPLGYHLPNVSLTFQAWHHLSDSERLCFLATTLRP | |
| | FPAMLGGLGTQGTWTSSEREQLWAMRLDLRDLHRHLRFQ | |
| | VLAAGFKCSKEEEDKEEEEEEEEEEKKLPLGALGGPNQVSS | |
| | QVSWPQLLYTYQLLHSLELVLSRAVRDLLLLSLPRRPGSAW | |
| | DS | |
| Mouse IL-27 | YTETALVALSQPRVQCHASRYPVAVDCSWTPLQAPNSTRST | 26 |
| EBI3 | SFIATYRLGVATQQQSQPCLQRSPQASRCTIPDVHLFSTVPY | |
| | MLNVTAVHPGGASSSLLAFVAERIIKPDPPEGVRLRTAGQRL | |
| | QVLWHPPASWPFPDIFSLKYRLRYRRRGASHFRQVGPIEATT | |
| | FTLRNSKPHAKYC | |
| Human IL-35 | RNLPVATPDPGMFPCLHHSQNLLRAVSNMLQKARQTLEFY | 27 |
| p35 | PCTSEEIDHEDITKDKTSTVEACLPLELTKNESCLNSRETSFI | |
| | TNGSCLASRKTSFMMALCLSSIYEDLKMYQVEFKTMNAKL | |
| | LMDPKRQIFLDQNMLAVIDELMQALNFNSETVPQKSSLEEP | |
| | DFYKTKIKLCILLHAFRIRAVTIDRVMSYLNAS | |
| Human IL-35 | RKGPPAALTLPRVQCRASRYPIAVDCSWTLPPAPNSTSPVSFI | 28 |
| Ebi3 | ATYRLGMAARGHSWPCLQQTPTSTSCTITDVQLFSMAPYV | |
| | LNVTAVHPWGSSSSFVPFITEHIIKPDPPEGVRLSPLAERQLQ | |
| | VQWEPPGSWPFPEIFSLKYWIRYKRQGAARFHRVGPIEATSF | |
| | ILRAVRPRARYYVQVAAQDLTDYGELSDWSLPATATMSLGK | |
| Mouse IL-35 | RVIPVSGPARCLSQSRNLLKTTDDMVKTAREKLKHYSCTAE | 29 |
| p35 | DIDHEDITRDQTSTLKTCLPLELHKNESCLATRETSSTTRGS | |
| | CLPPQKTSLMMTLCLGSIYEDLKMYQTEFQAINAALQNHN | |
| | HQQIILDKGMLVAIDELMQSLNHNGETLRQKPPVGEADPYR | |
| | VKMKLCILLHAFSTRVVTINRVMGYLSSA | |
| Mouse IL-35 | ALVALSQPRVQCHASRYPVAVDCSWTPLQAPNSTRSTSFIAT | 30 |
| Ebi3 | YRLGVATQQQSQPCLQRSPQASRCTIPDVHLFSTVPYMLNV | |
| | TAVHPGGASSSLLAFVAERIIKPDPPEGVRLRTAGQRLQVLW | |
| | HPPASWPFPDIFSLKYRLRYRRRGASHFRQVGPIEATTFTLR | |
| | NSKPHAKYCIQVSAQDLTDYGKPSDWSLPGQVESAPHKP | |
| Human IL- | VLSGALCFRMKDSALKVLYLHNNQLLAGGLHAGKVIKGEE | 31 |
| 36ra | ISVVPNRWLDASLSPVILGVQGGSQCLSCGVGQEPTLTLEP | |
| | VNIMELYLGAKESKSFTFYRRDMGLTSSFESAAYPGWFLCT | |
| | VPEADQPVRLTQLPENGGWNAPITDFYFQQCD | |
| Mouse IL-36ra | VLSGALCFRMKDSALKVLYLHNNQLLAGGLHAEKVIKGEE | 32 |
| | ISVVPNRALDASLSPVILGVQGGSQCLSCGTEKGPILKLEPV | |
| | NIMELYLGAKESKSFTFYRRDMGLTSSFESAAYPGWFLCTS | |
| | PEADQPVRLTQIPEDPAWDAPITDFYFQQCD | |
| Human IL-37 | VHTSPKVKNLNPKKFSIHDQDHKVLVLDSGNLIAVPDKNYI | 33 |
| | RPEIFFALASSLSSASAEKGSPILLGVSKGEFCLYCDKDKGQ | |
| | SHPSLQLKKEKLMKLAAQKESARRPFIFYRAQVGSWNMLE | |
| | SAAHPGWFICTSCNCNEPVGVTDKFENRKHIEFSFQPVCKA | |
| | EMSPSEVSD | |
| Mouse IL-37 | VPSSAKVNARAPEKFSIRDQDQKVLVLSDETLIAVPNKPYT | 34 |
| | VPETFFVLASHTSSSHEGSPILLAVSKGELCLCCDKDEEQSK | |
| | PSLQLKKNELMKLATQKEKVRLPFVFYRAQVGSCCTLESA | |
| | AHPGWFVCTSRNSGAPVEVTDTSGEGKLMEFSFQQVSETE | |
| | MSPSEVSI | |
| Human | SYNLLGFLQRSSNFQCQKLLWQLNGRLEYCLKDRMNFDIP | 35 |
| interferon-ß | EEIKQLQQFQKEDAALTIYEMLQNIFAIFRQDSSSTGWNETI | |
| | VENLLANVYHQINHLKTVLEEKLEKEDFTRGKLMSSLHLK | |
| | RYYGRILHYLKAKEYSHCAWTIVRVEILRNFYFINRLTGYLR | |
| | N | |
| Mouse | INYKQLQLQERTNIRKCQELLEQLNGKINLTYRADFKIPME | 36 |
| interferon-ß | MTEKMQKSYTAFAIQEMLQNVFLVFRNNFSSTGWNETIVV | |
| | RLLDELHQQTVFLKTVLEEKQEERLTWEMSSTALHLKSYY | |
| | WRVQRYLKLMKYNSYAWMVVRAEIFRNFLIIRRLTRNFQN | |
| Human TGF- | ALDTNYCFSSTEKNCCVRQLYIDFRKDLGWKWIHEPKGYH | 37 |
| ß1 | ANFCLGPCPYIWSLDTQYSKVLALYNQHNPGASAAPCCVP | |
| | QALEPLPIVYYVGRKPKVEQLSNMIVRSCKCS | |
| Mouse TGF-ß1 | ALDTNYCFSSTEKNCCVRQLYIDFRKDLGWKWIHEPKGYH | 38 |
| | ANFCLGPCPYIWSLDTQYSKVLALYNQHNPGASASPCCVP | |
| | QALEPLPIVYYVGRKPKVEQLSNMIVRSCKCS | |
| Human IL-38 | MCSLPMARYYIIKYADQKALYTRDGQLLVGDPVADNCCAE | 55 |
| | KICILPNRGLARTKVPIFLGIQGGSRCLACVETEEGPSLQLED | |
| | VNIEELYKGGEEATRFTFFQSSSGSAFRLEAAAWPGWFLCG | |
| | PAEPQQPVQLTKESEPSARTKFYFEQSW | |

Certain anti-inflammatory cytokines are further described in, for example, Opal SM, DePalo VA. Chest. 2000 April; 117(4):1162-72, incorporated herein by reference in its entirety.

C. Albumin Proteins

Aspects of the disclosure are directed to albumin proteins, polypeptides linked to albumin proteins, and polypeptides comprising an albumin protein. In some aspects, an albumin protein is human albumin (also “human serum albumin,” or “HSA,”). Human albumin is identified at NCBI reference sequence NM_000477.7. In some aspects, an albumin protein is a mouse albumin (also “mouse serum albumin,” or “MSA”). Mouse albumin is identified at NCBI reference sequence NM_009654.4. In some aspects, an albumin protein of the present disclosure is a fully processed albumin protein which does not comprise a signal peptide and/or a propeptide. Certain, non-limiting examples of albumin proteins contemplated herein are provided in Table 3.

TABLE 3

| Albumin | | SEQ ID |
|---|---|---|
| Protein | Sequence | NO: |
| Human | DAHKSEVAHRFKDLGEENFKALVLIAFAQYLQQCPFEDHV | 1 |
| Albumin (fully | KLVNEVTEFAKTCVADESAENCDKSLHTLFGDKLCTVATLR | |
| processed) | ETYGEMADCCAKQEPERNECFLQHKDDNPNLPRLVRPEVD | |
| | VMCTAFHDNEETFLKKYLYEIARRHPYFYAPELLFFAKRYK | |
| | AAFTECCQAADKAACLLPKLDELRDEGKASSAKQRLKCAS | |
| | LQKFGERAFKAWAVARLSQRFPKAEFAEVSKLVTDLTKVHT | |
| | ECCHGDLLECADDRADLAKYICENQDSISSKLKECCEKPLL | |
| | EKSHCIAEVENDEMPADLPSLAADFVESKDVCKNYAEAKD | |
| | VFLGMFLYEYARRHPDYSVVLLLRLAKTYETTLEKCCAAA | |
| | DPHECYAKVFDEFKPLVEEPQNLIKQNCELFEQLGEYKFQN | |
| | ALLVRYTKKVPQVSTPTLVEVSRNLGKVGSKCCKHPEAKR | |
| | MPCAEDYLSVVLNQLCVLHEKTPVSDRVTKCCTESLVNRR | |
| | PCFSALEVDETYVPKEFNAETFTFHADICTLSEKERQIKKQT | |
| | ALVELVKHKPKATKEQLKAVMDDFAAFVEKCCKADDKET | |
| | CFAEEGKKLVAASQAALGL | |
| Mouse | EAHKSEIAHRYNDLGEQHFKGLVLIAFSQYLQKCSYDEHA | 2 |
| Albumin (fully | KLVQEVTDFAKTCVADESAANCDKSLHTLFGDKLCAIPNLR | |
| processed) | ENYGELADCCTKQEPERNECFLQHKDDNPSLPPFERPEAEA | |
| | MCTSFKENPTTFMGHYLHEVARRHPYFYAPELLYYAEQYN | |
| | EILTQCCAEADKESCLTPKLDGVKEKALVSSVRQRMKCSSM | |
| | QKFGERAFKAWAVARLSQTFPNADFAEITKLATDLTKVNKE | |
| | CCHGDLLECADDRAELAKYMCENQATISSKLQTCCDKPLL | |
| | KKAHCLSEVEHDTMPADLPAIAADFVEDQEVCKNYAEAKD | |
| | VFLGTFLYEYSRRHPDYSVSLLLRLAKKYEATLEKCCAEAN | |
| | PPACYGTVLAEFQPLVEEPKNLVKTNCDLYEKLGEYGFQNA | |
| | ILVRYTQKAPQVSTPTLVEAARNLGRVGTKCCTLPEDQRLP | |
| | CVEDYLSAILNRVCLLHEKTPVSEHVTKCCSGSLVERRPCFS | |
| | ALTVDETYVPKEFKAETFTFHSDICTLPEKEKQIKKQTALAE | |
| | LVKHKPKATAEQLKTVMDDFAQFLDTCCKAADKDTCFSTE | |
| | GPNLVTRCKDALA | |

D. Albumin Binding Proteins

Aspects of the disclosure are directed to albumin binding proteins, polypeptides linked to albumin binding proteins, and polypeptides comprising an albumin protein. An “albumin binding protein” describes a protein capable of binding to an albumin protein (e.g., human albumin). In some aspects, an albumin binding protein is an anti-albumin antibody or antibody-like molecule. In some aspects, an albumin binding protein is a polypeptide comprising the sequence DICLPRWGCLW (SEQ ID NO:51).

E. Detection Peptides

In some aspects, the polypeptides described herein may further comprise a detection peptide (also “tag”). Suitable detection peptides include hemagglutinin (HA; e.g., YPYDVPDYA (SEQ ID NO:39); FLAG (e.g., DYKDDDDK (SEQ ID NO:40); c-myc (e.g., EQKLISEEDL; SEQ ID NO:41), His (e.g., HHHHHH; SEQ ID NO:42), and the like. In some aspects, a polypeptide described herein comprises a tag sequence having at least 75%, at least 80%, at least 85%, at least 90%, at least 95%, at least 98%, or 100% amino acid sequence identity to SEQ ID NO:39, SEQ ID NO:40, SEQ ID NO:41, or SEQ ID NO:42. Other suitable detection peptides are known in the art.

F. Peptide Linkers

In some aspects, the polypeptides of the disclosure include peptide linkers (sometimes referred to as a linker). A peptide linker may be used to separate any of the peptide domain/regions described herein. As an example, a linker may be between the albumin protein and the anti-inflammatory cytokine, between the anti-inflammatory cytokine and a detection peptide or tag, at an N-terminus of a polypeptide, and/or at a C-terminus of a polypeptide. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length, or any value or range derivable therein. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers (including, for example, (GS)n, (GSGGS)n (SEQ ID NO:145), (G4S)n and (GGGS)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGGS (SEQ ID NO:43), GGSG (SEQ ID NO:44), GGSGG (SEQ ID NO:45), GSGSG (SEQ ID NO:46), GSGGG (SEQ ID NO:47), GGGSG (SEQ ID NO:48), GSSSG (SEQ ID NO:49), GGGSGGGS (SEQ ID NO:50), and the like.

In further aspects, the linker comprises (EAAAK)n, wherein n is an integer of at least one. In some aspects, n is at least, at most, or exactly 1, 2, 3, 4, 5, 6, 7, 8, 9, or 10 (or any derivable range therein).

### II. NUCLEIC ACIDS

In certain aspects, nucleic acid sequences can exist in a variety of instances such as: isolated segments and recombinant vectors of incorporated sequences or recombinant polynucleotides encoding an anti-inflammatory polypeptide, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding anti-inflammatory polypeptides are provided in certain aspects. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### III. IMMUNOTHERAPY

In some aspects, the methods comprise administration of a cancer immunotherapy and/or the subject is one that is being treated with an immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

A. Immune Checkpoint Blockade Therapy

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the ICB therapy comprises a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the ICB therapy comprises a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the ICB therapy comprises an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as an ICB therapy in the methods and compositions of the disclosure is ipilimumab (also known as 10DI, MDX-010, MDX-101, and Yervoy®) or antigen binding fragments and variants thereof (see, e.g., WOO 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

B. Activation of Co-Stimulatory Molecules

In some aspects, the immunotherapy comprises an activator of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

F. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.[60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### IV. THERAPEUTIC METHODS

The compositions of the disclosure may be used for in vivo, in vitro, or ex vivo administration. The route of administration of the composition may be, for example, intracutaneous, subcutaneous, intravenous, intradermal, intramuscular, local, topical, and/or intraperitoneal administrations. It is specifically contemplated that one or more of these routes of administration are excluded from certain aspects of the disclosure.

In some aspects, a composition of the disclosure is provided via subcutaneous administration (i.e., is provided subcutaneously). In some aspects, a composition of the disclosure is provided via intradermal administration (i.e., is provided intradermally). In some aspects, a composition of the disclosure is provided via intramuscular administration (i.e., is provided intramuscularly). In some aspects, a composition of the disclosure is provided at a site of a wound. In some aspects, a composition of the disclosure is not provided at a site of a wound (e.g., is provided at a site different from the site of the wound).

In some aspects, a therapeutic composition of the disclosure is administered during the cessation of one or more other therapies. For example, in some aspects, disclosed is a method comprising administering to a subject an anti-inflammatory polypeptide (e.g., an anti-inflammatory cytokine linked to an albumin protein) during cessation of an additional anti-inflammatory therapeutic (e.g., fingolimod, interferon-β, dimethyl fumarate, teriflunomide, integrin α4β1, an anti-αLβ2 antibody, an anti-TNFα agent, an anti-IL-6R agent, an anti-IL-6 agent, or a Janus kinase inhibitor.).

A. Autoimmune or Inflammatory Conditions

Aspects of the present disclosure are directed to methods for treating autoimmune or inflammatory conditions. In some aspects, disclosed herein is a method for treating an autoimmune or inflammatory condition comprising administering to a subject a composition comprising an anti-inflammatory cytokine (e.g., IL-4, IL-10, IL-33, IL-35, etc.), where the subject has, is at risk for developing, or is suspected of having an autoimmune or inflammatory condition. Such methods may comprise administrating one or more additional anti-inflammatory agents. Such methods may exclude administering one or more additional anti-inflammatory agents. Additional anti-inflammatory agents include, for example, fingolimod, interferon-β, dimethyl fumarate, teriflunomide, integrin α4β1, an anti-αLβ2 antibody, an anti-TNFα agent, an anti-IL-6R agent, an anti-IL-6 agent, and a Janus kinase inhibitor (e.g., tofacitinib, baricitinib, upadacitinib).

The autoimmune or inflammatory condition (also “disease” or “disorder”) amenable for treatment may include, but not be limited to, conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, endometriosis, and cytokine storm syndrome (also “cytokine release syndrome”) such as cytokine storm syndrome caused by cancer immunotherapy or viral infection (e.g., SARS-CoV-2 infection). It is contemplated that one or more of these conditions or diseases may be excluded in an aspect disclosed herein.

In some aspects, the disclosed methods are for treating a subject for multiple sclerosis. A method for treating a subject for multiple sclerosis may comprise administering to the subject IL-4 linked to an albumin protein. A method for treating a subject for multiple sclerosis may comprise administering to the subject IL-33 linked to an albumin protein.

In some aspects, the disclosed methods are for treating a subject for arthritis. In some aspects, the disclosed methods are for treating a subject for rheumatoid arthritis. A method for treating a subject for arthritis may comprise administering to the subject IL-10 linked to an albumin protein. A method for treating a subject for arthritis may comprise administering to the subject IL-35 linked to an albumin protein.

In some aspects, the disclosed methods are for treating a subject for type 1 diabetes. In some aspects, the disclosed methods are for treating a subject for diabetic peripheral neuropathy. In some aspects, the disclosed methods are for treating a subject for psoriasis. In some aspects, the disclosed methods are for treating a subject for inflammatory bowel disease. In some aspects, the disclosed methods are for treating a subject for Crohn's disease. In some aspects, the disclosed methods are for treating a subject for systemic scleroderma. In some aspects, the disclosed methods are for treating a subject for cytokine storm syndrome (including, for example, cytokine storm syndrome caused by cancer immunotherapy and cytokine storm syndrome caused by a viral infection such as SARS-CoV-2 infection). In some aspects, the disclosed methods are for treating a subject for acute respiratory distress syndrome (ARDS).

B. Wound Healing

Aspects of the present disclosure are directed to methods for promoting wound healing. In some aspects, disclosed herein is a method for promoting wound healing comprising administering to a subject a composition comprising an anti-inflammatory cytokine (e.g., IL-4, IL-10, IL-33, IL-35, etc.) operatively linked to an albumin protein, where the subject has a wound. In some aspects, the wound is a chronic wound. In some aspects, the wound is a diabetic ulcer. In some aspects, disclosed is a method for promoting wound healing comprising administering to a subject a composition comprising IL-4 operatively linked to an albumin protein. Such methods may comprise administrating one or more additional wound healing agents.

C. Anti-Inflammatory Cytokine Targeting

Aspects of the present disclosure are directed to methods for targeting an anti-inflammatory cytokine to a lymph node of a subject. In some aspects, an anti-inflammatory cytokine is targeted to a lymph node of a subject by linking the cytokine to an albumin protein or an albumin binding protein. The linked polypeptides may then be administered to a subject to target the anti-inflammatory cytokine to a lymph node of the subject. In some aspects, following administration, the anti-inflammatory cytokine remains in the lymph node at least 0.5, 1, 2, 3, 4, 5, 6, 7, 8, 9, 10, 11, 12, 13, 14, 15, 16, 17, 18, 19, 20, 21, 22, 23, or 24 hours (or any range derivable therein), or more, after administering the composition to the subject.

D. Administration of Therapeutic Compositions

Aspects of the disclosure relate to compositions and methods comprising therapeutic compositions. Different therapies may be administered in one composition or in more than one composition, such as 2 compositions, 3 compositions, or 4 compositions. Various combinations of agents may be employed.

The therapeutic agents of the disclosure (e.g., anti-inflammatory polypeptides, anti-inflammatory cytokines) may be administered by one or more routes of administration. In some aspects, the therapeutic agent is administered intravenously, intramuscularly, subcutaneously, topically, orally, transdermally, intraperitoneally, intraorbitally, by implantation, by inhalation, intrathecally, intraventricularly, or intranasally. It is specifically contemplated that one or more of these routes of administration are excluded from certain aspects of the disclosure. In some aspects, the therapeutic agent is administered subcutaneously. In some aspects, the therapeutic agent is administered intramuscularly. In some aspects, the therapeutic agent is administered intradermally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose (also “effective amount”) is understood to refer to an amount necessary to achieve a particular effect. In the practice in certain aspects, it is contemplated that doses in the range from 0.1 mg/kg to 50 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

E. Treating a Subject with Cancer

In aspects of the disclosure, the polypeptides are used for treating subjects having cancer. The cancer may include, but is not limited to, tumors of all types, locations, sizes, and characteristics. In some aspects, the cancer comprises a solid tumor. In some aspects, the cancer comprises, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, lung cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and Wilms tumor (kidney cancer).

### V. EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Prolonged Residence of Albumin-Fused IL-4 in the Secondary Lymphoid Organs Ameliorates Experimental Autoimmune Encephalomyelitis

A. Results

1. SA-IL-4 Binds to Immune Cells and Inhibits Th17 Differentiation

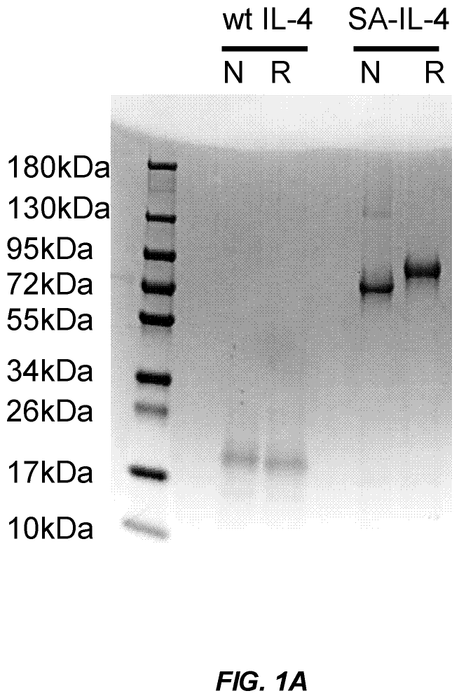

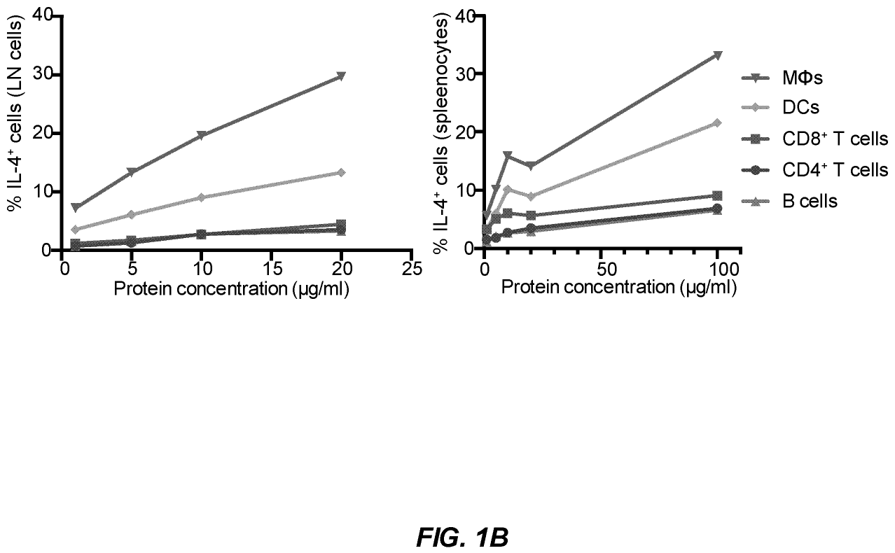

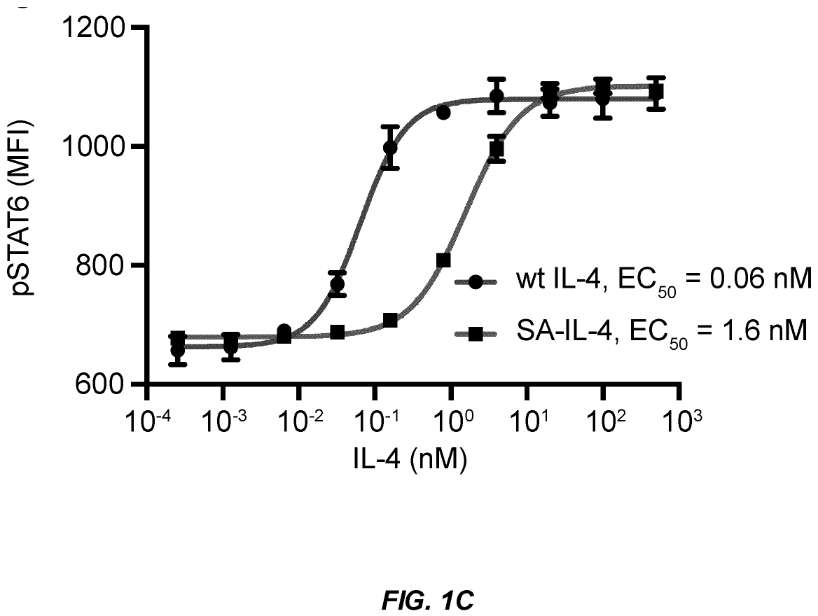

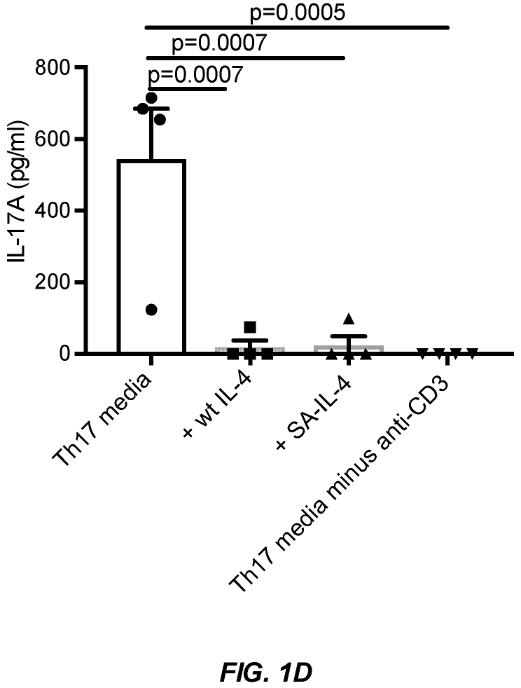

Wild-type (wt) mouse IL-4 and mouse serum albumin (SA)-fused mouse IL-4 were recombinantly expressed (FIG. 1A). SDS-PAGE revealed that the molecular size was increased by SA fusion to IL-4. When added to freshly isolated immune cells from lymph node (LN) and spleen, SA-IL4 preferentially bound to antigen presenting cells (APCs), such as macrophages and dendritic cells (DCs) in vitro, compared to other immune cells (FIG. 1).

IL-4 receptor is known to be expressed on T cells when stimulated¹³. SA-IL-4 induced downstream phosphorylation of STAT6 in T cells with 32 times higher EC50 than wt IL-4. This suggested that wt IL-4 is more active than SA-IL-4 in vitro (FIG. 1C). However, although STAT6 phosphorylation was reduced with the engineered IL-4, it was found that both wt IL-4 and SA-IL-4 were equivalently effective in inhibiting Th17 differentiation of naïve CD4⁺ T cells cultured in Th17 cell differentiation media (FIG. 1D). Taken together, the results demonstrated that the inventors successfully made functionally active SA-IL-4 fusion protein.

2. SA-IL-4 Increased Blood Half-Life and Persistence in the SLOs, Both in LNs and Spleen

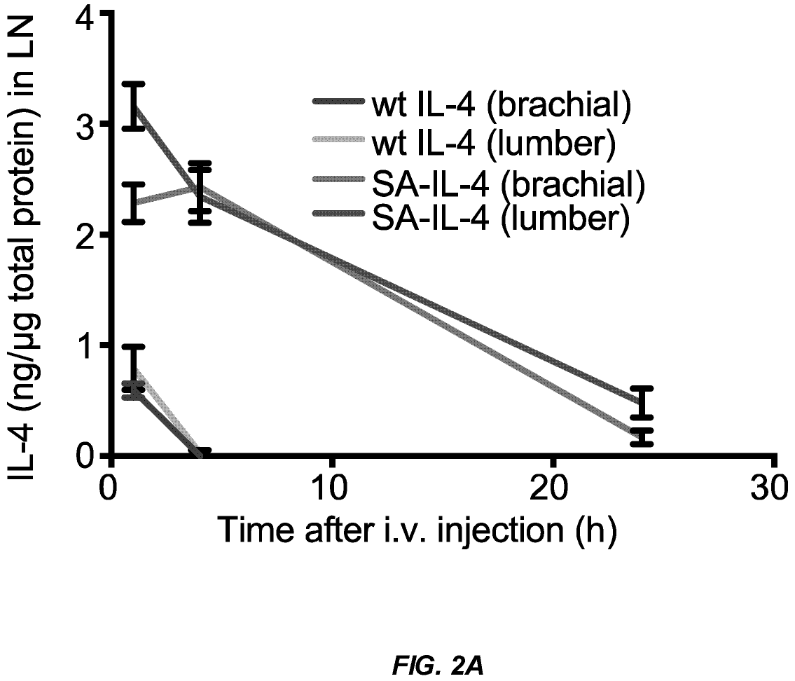

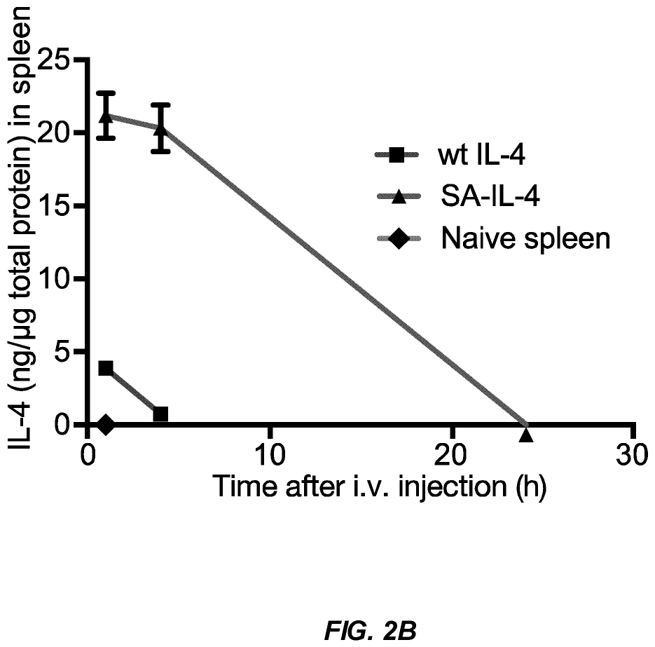

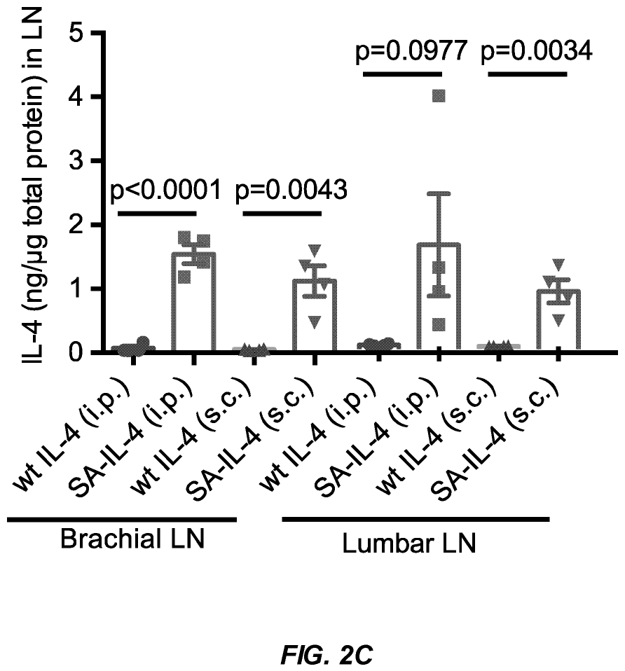

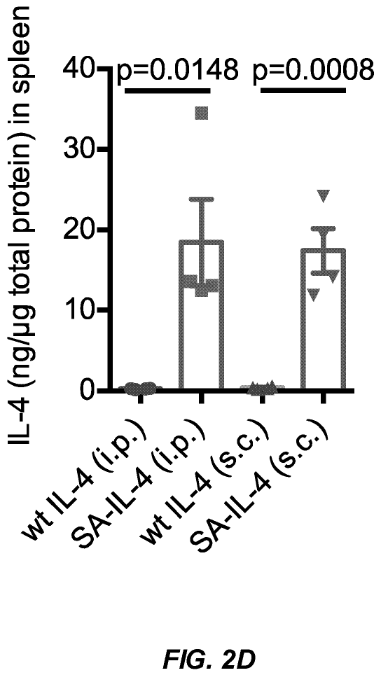

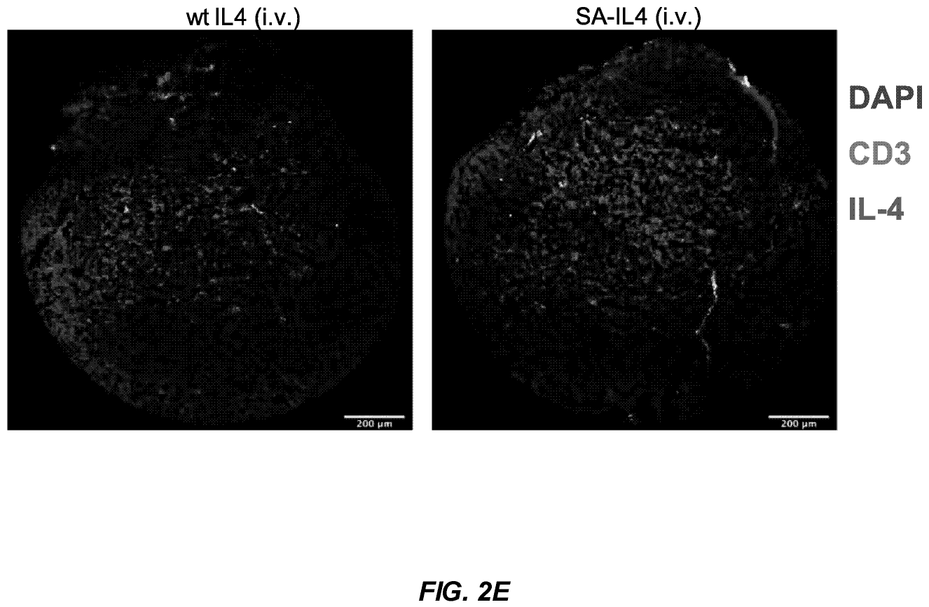

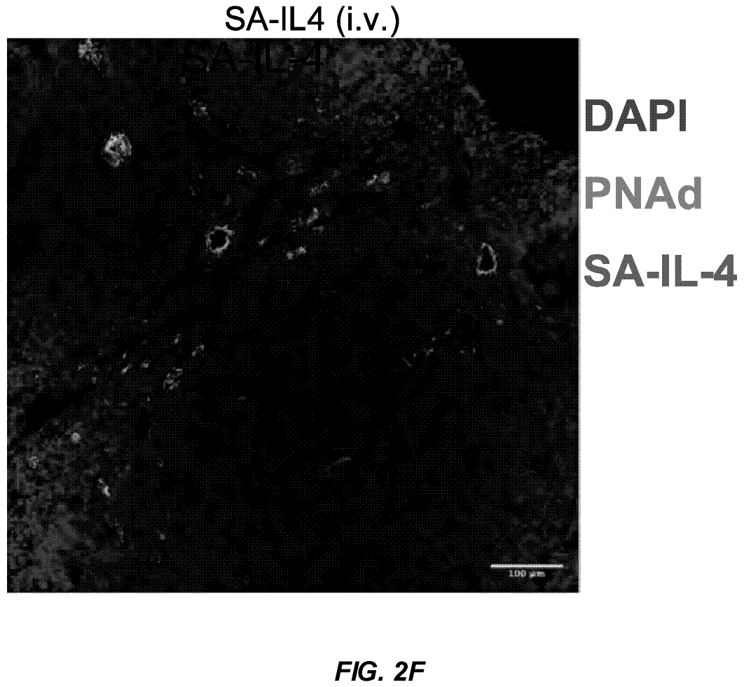

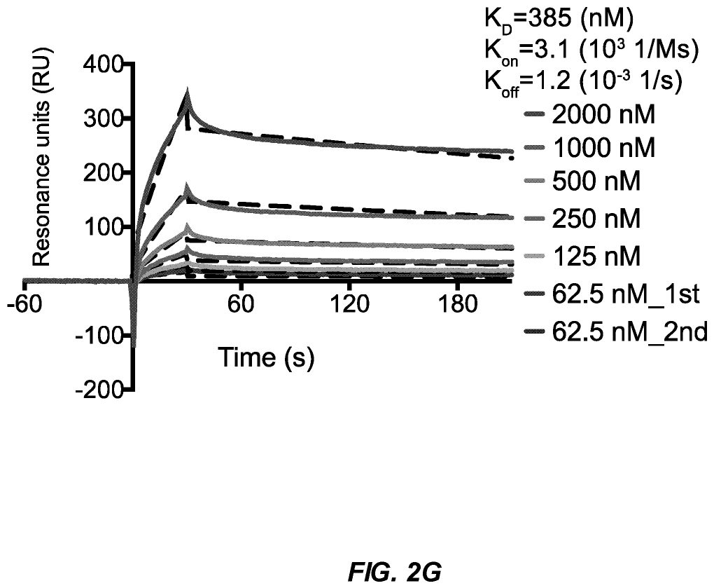

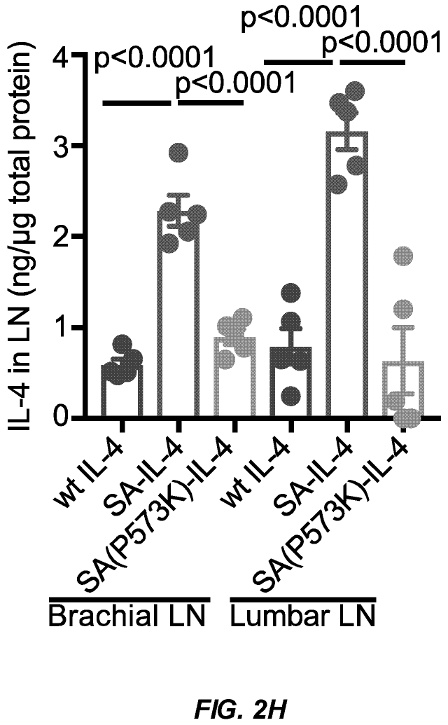

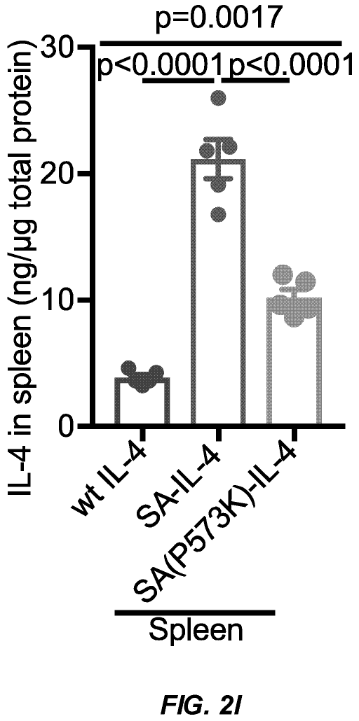

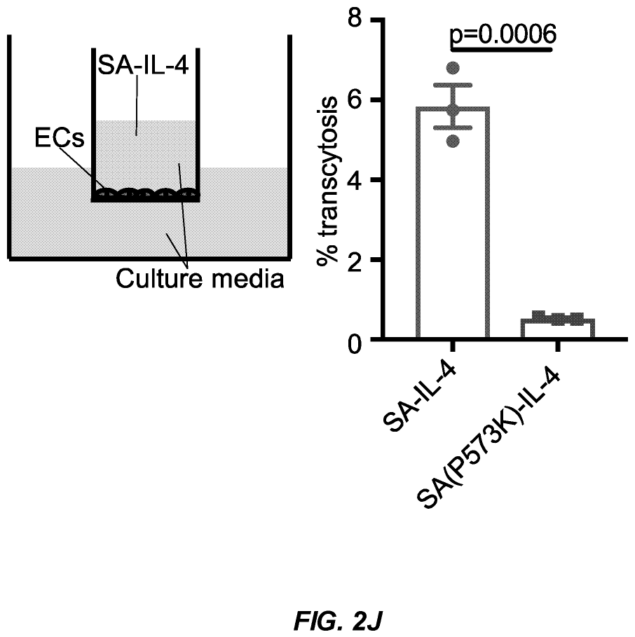

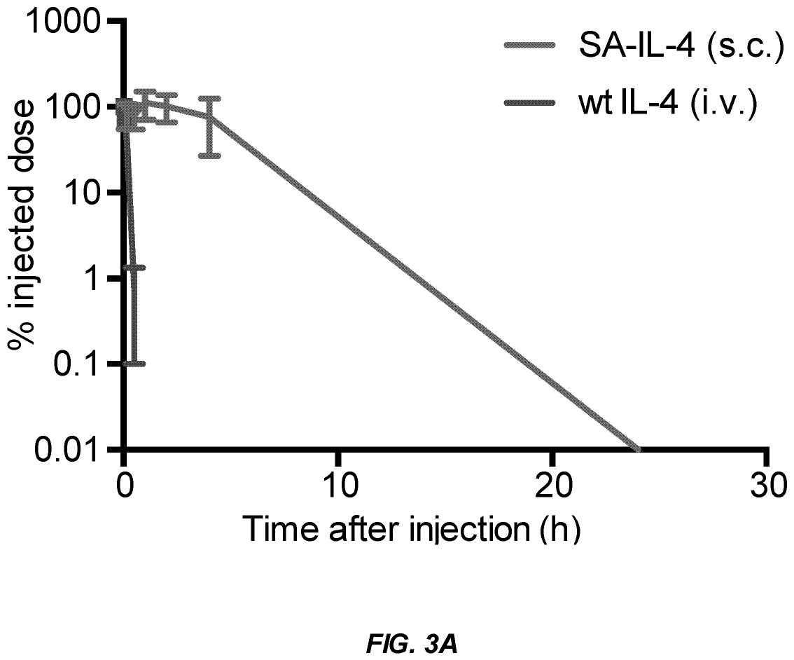

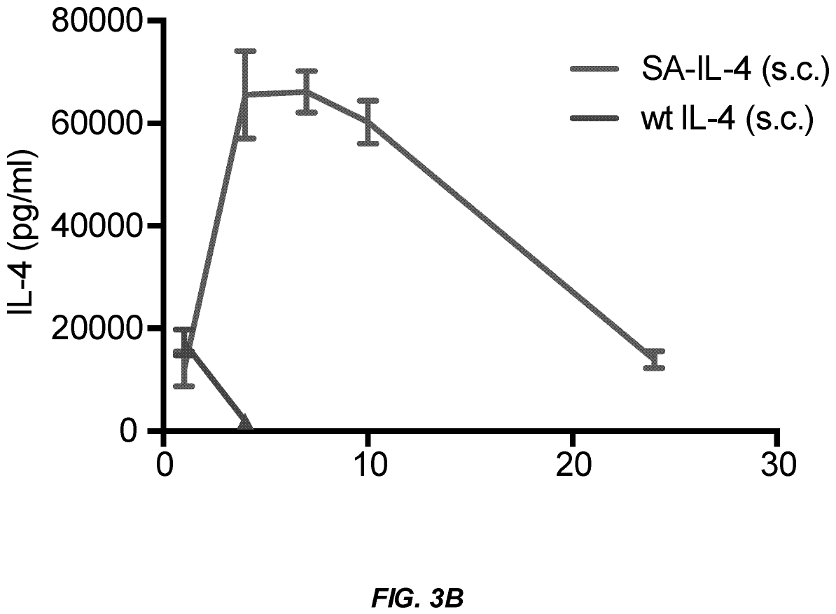

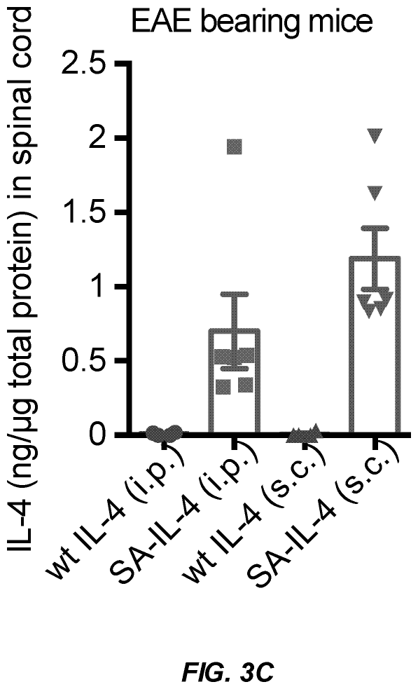

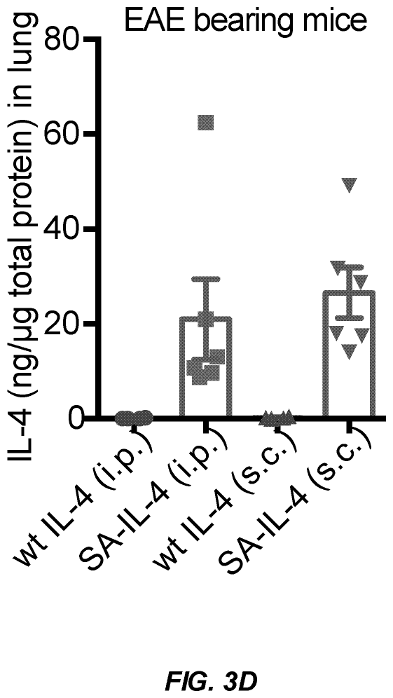

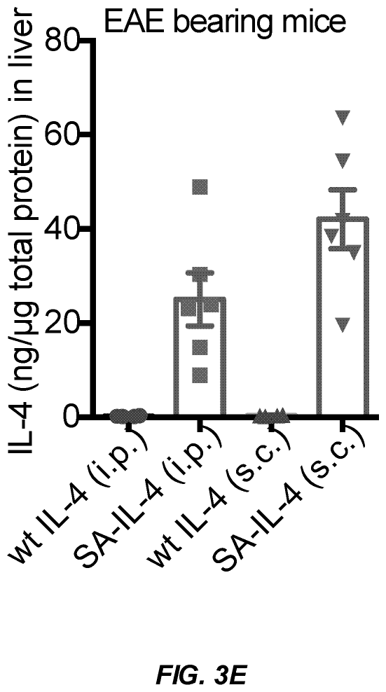

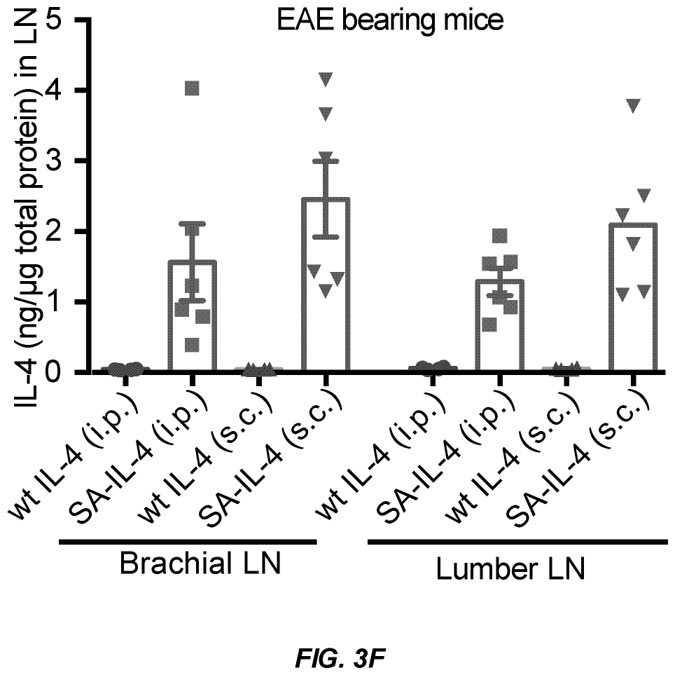

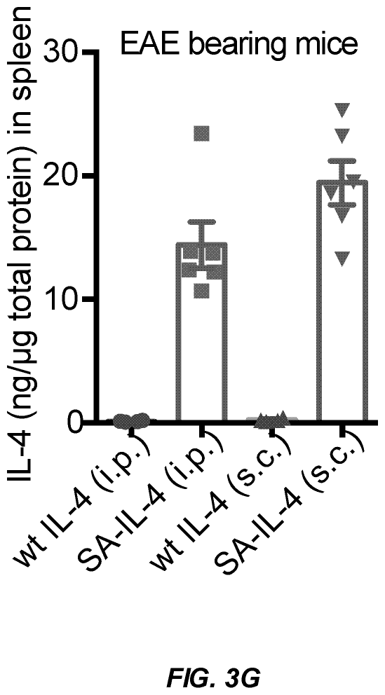

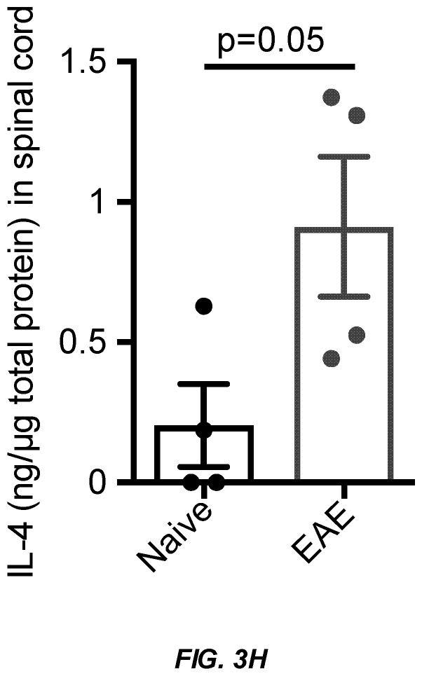

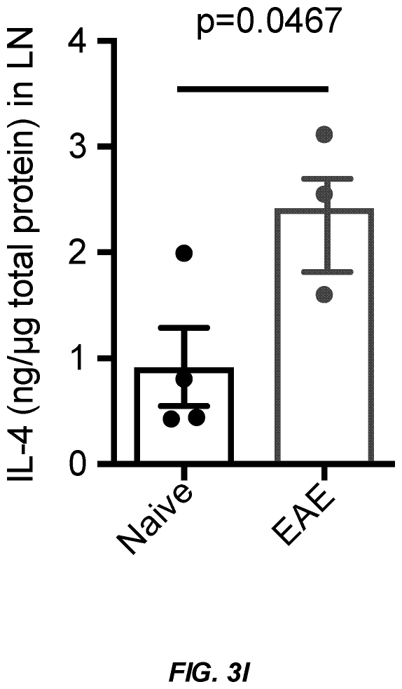

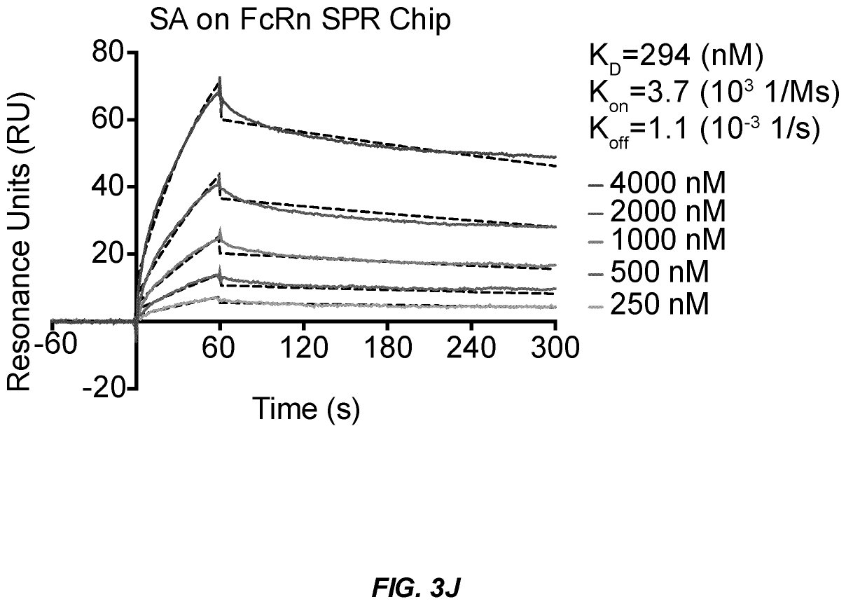

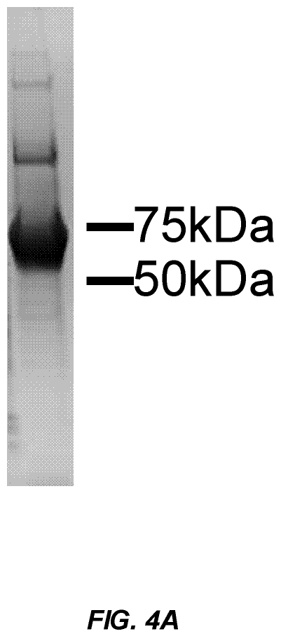

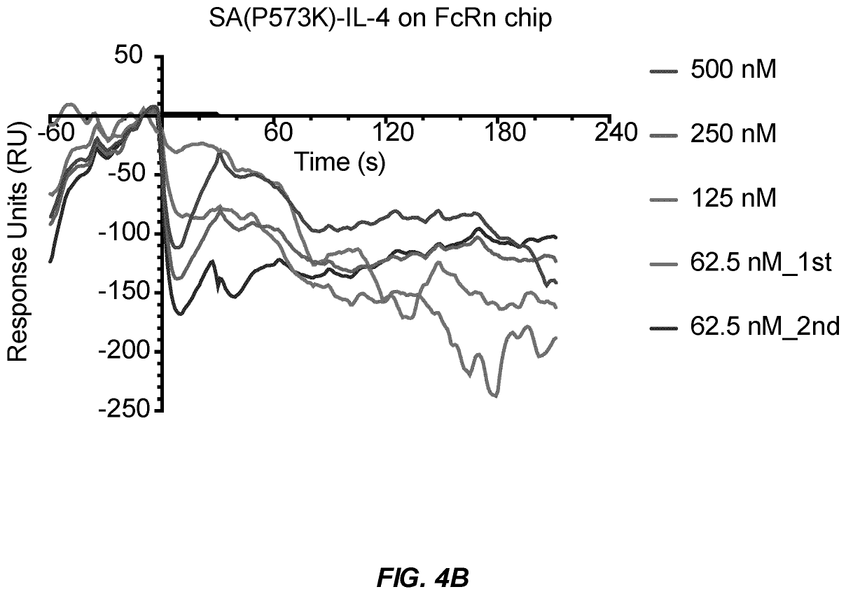

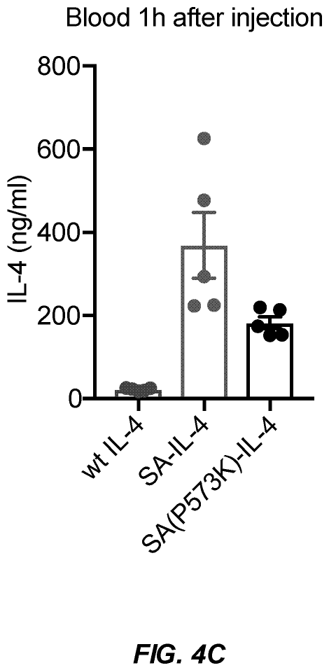

The inventors tested whether intravenous (i.v.) injection of SA-IL-4 caused accumulation in the spleen and LN using naïve mice. SA fusion to IL-4 substantially increased the amount of IL-4 in both the lumbar and brachial LNs and in the spleen after i.v. injection (FIGS. 2A and 2B). In addition, subcutaneously (s.c.) and intraperitoneally (i.p.) injected SA-IL-4 showed markedly higher amounts in both the lumbar and brachial LNs and in the spleen (FIGS. 2C and 2D). The amount of SA-IL-4 was also increased in various organs, such as liver and lung, possibly due to extended plasma half-life (FIGS. 3A-3G). EAE mice showed higher accumulation of SA-IL-4 in the lumbar LN and spinal cord (FIGS. 3J-3I). Fluorescence-based biodistribution analysis also showed enhanced SA-IL-4 accumulation in the lumbar LN compared to wt IL-4. Surface plasmon resonance (SPR) analysis showed that SA-IL-4 bound to FcRn with a dissociation constant (K_{D}) of 385 nM (FIG. 2E), which was similar to the affinity of SA to FcRn (FIG. 3J). Plasma half-life after i.v. and subcutaneous (s.c.) injection of SA-IL-4 was extended substantially compared to wt IL-4, which was cleared from plasma within a few minutes (FIGS. 3A and 3B). To test the involvement of FcRn in SA-IL-4 accumulation in the LN, the inventors made a P573K point mutation of SA, which abolishes FcRn binding (FIGS. 4A and 4B)¹⁴. The SA(P573K) mutation decreased the amount of IL-4 in the LN compared to SA-IL-4, down to a similar level as wt IL-4 (FIG. 2E). SA(P573K)-IL-4 had a longer plasma half-life compared to wt IL-4, likely due to an increase in molecular size, but a shorter half-life than SA-IL-4 due to impaired FcRn binding (FIG. 4C). It was also verified that FcRn is expressed in a variety of cells from LNs, liver, and lung, suggesting that LN localization of SA-IL-4 is not due to FcRn specific expression in the LN. Taken together, these data suggested that SA trafficking to the LN required FcRn binding. The SA(P573K)-IL-4 amount in the spleen was lower than with SA-IL-4 (FIG. 2F).

It was hypothesized that transcytosis by blood endothelial cells was inhibited by the SA(P573K) mutation, resulting in reduced trafficking of SA-IL-4 to the LN. To test this, the inventors performed a transcytosis assay using human endothelial cells cultured on the cell culture insert (FIG. 2G). The SA(P573K) mutation significantly reduced SA-IL-4 transcytosis by endothelial cells. Importantly, mouse SA binds to human FcRn¹⁵ and mouse IL-4 does not bind to human IL-4Rα¹⁶, thus the inventors could test FcRn-mediated transcytosis in this assay. Taken together, it was shown that SA fusion to IL-4 increases persistence of IL-4 within the LN and spleen at least in part through FcRn binding.

3. SA-IL-4 Treatment Strongly Suppressed EAE Disease Development in Prophylactic Treatment

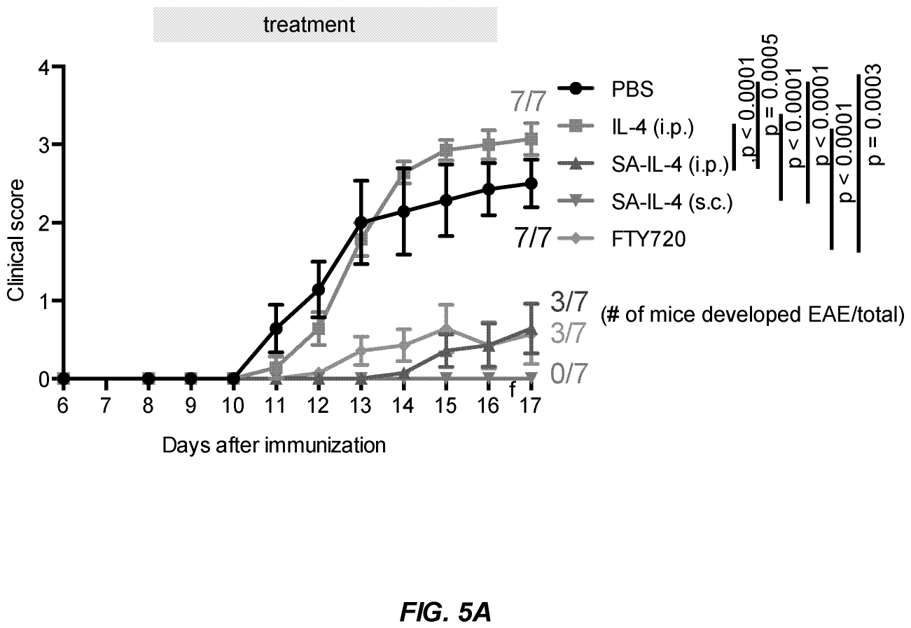

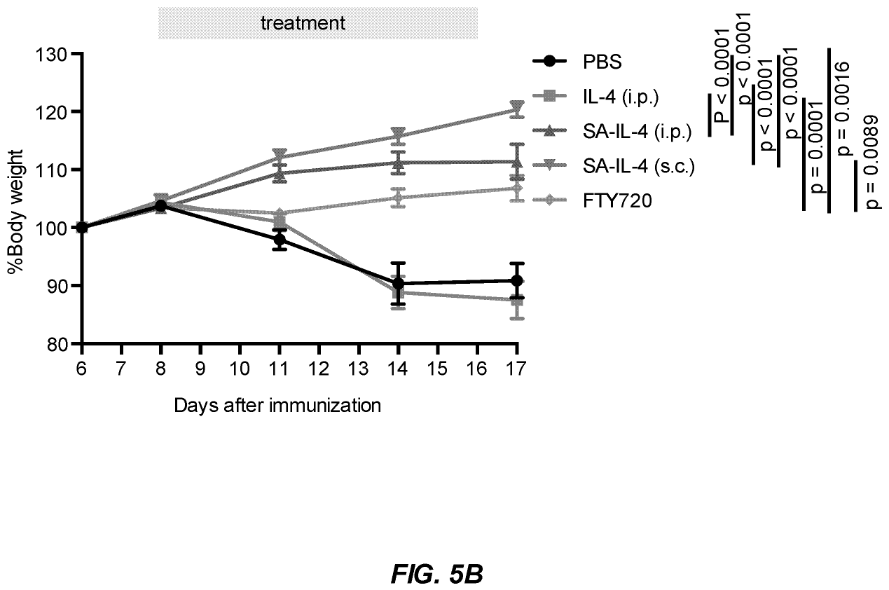

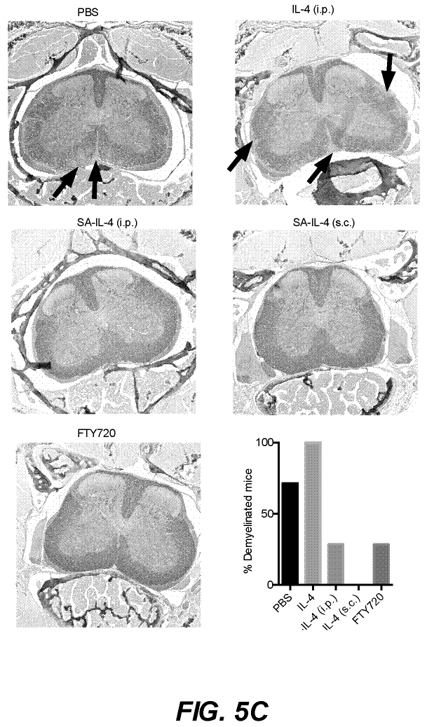

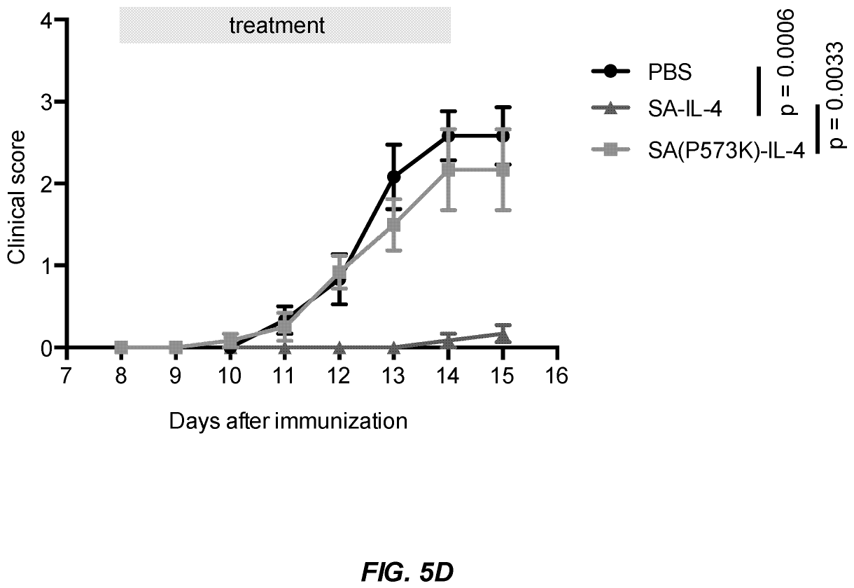

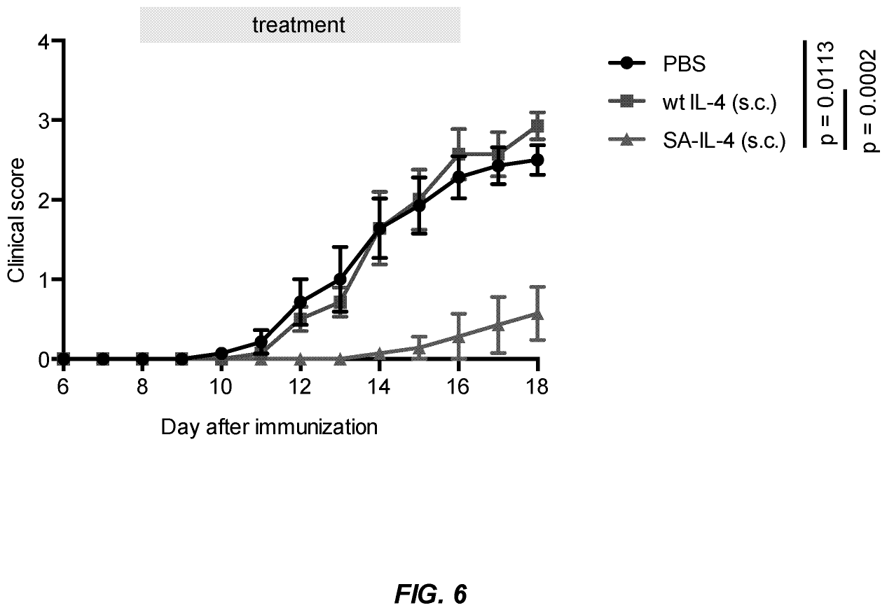

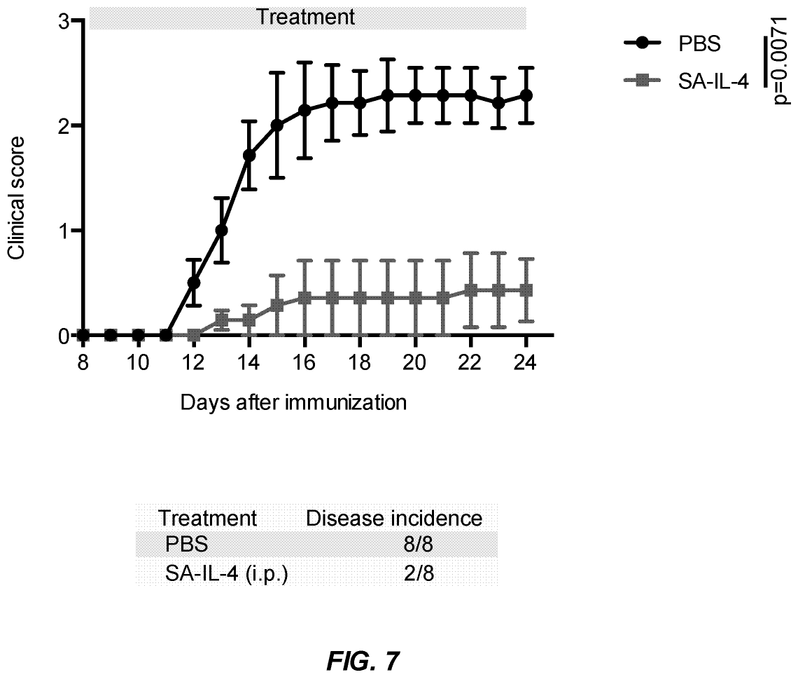

The inventors next treated myelin oligodendrocyte glycoprotein (MOG) antigen-induced EAE, in which disease is induced by administration of MOG₃₅₋₅₅ in complete Freund's adjuvant, with SA-IL-4 in the acute phase of EAE (FIGS. 5A-5D). SA-IL-4 was injected s.c. or i.p. S.c. injection was chosen because it is clinically convenient. Injection i.p. was done as a surrogate for i.v. because the tail becomes flaccid in mice that have developed EAE, and tail vein injection thus becomes difficult. The inventors further compared the therapeutic effect of SA-IL-4 with FTY720⁴. Although, there was no statistical difference among SA-IL-4 (both i.p. and s.c. injections) and FTY720 groups in terms of clinical score, s.c. injection of SA-IL-4 completely suppressed disease development in all mice (FIG. 5A). SA-IL-4 injected i.p. and FTY720 prevented EAE development in 4 of 7 mice and suppressed disease severity in the rest. Wt IL-4 treatment did not show EAE clinical score suppression compared to the PBS treated group, all of which mice developed disease. Using body weight changes as a clinical indicator of health, mice treated with PBS and wt IL-4 were observed to markedly lose weight (FIG. 5B). Mice injected s.c. with SA-IL-4 gained more weight than all other groups, indicating good health. The i.p. SA-IL-4 group gained weight on average, while FTY720 treated mice maintained their body weight. Next, demyelination of the spinal cord was analyzed, which is the main histomorphological manifestation of EAE disease (FIG. 5C). Mice injected s.c. with SA-IL-4 did not have any detectable demyelination, demonstrating prevention of damage in the spinal cord. All wt IL-4-treated mice showed demyelination. Mice were then monitored long-term, until day 24, where SA-IL-4 i.p. injection inhibited disease development and progression (FIG. 6). It was confirmed that wt IL-4 did not show a marked therapeutic effect even when injected subcutaneously (FIG. 7). It was also found that introducing the SA(P573K) mutation abolished the therapeutic effect of SA-IL-4, suggesting that FcRn binding of SA-IL-4 plays a critical role for EAE therapy. These data demonstrated that SA fusion to IL-4 strongly improved the therapeutic effects of IL-4 on EAE disease suppression.

4. SA-IL-4 Treatment Suppressed Immune Cell Infiltration into the Spinal Cord and Induced an Immune-Suppressive Environment in the dLN

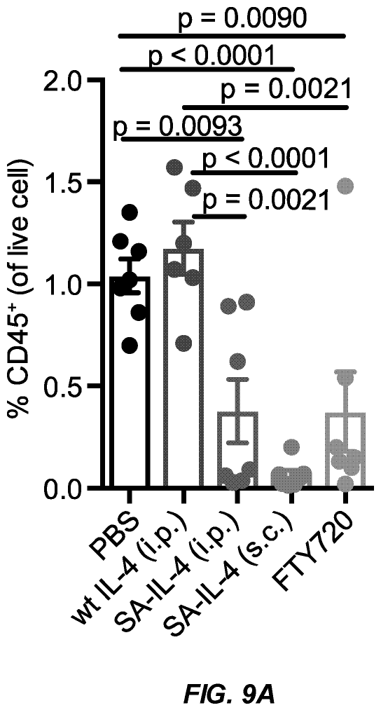

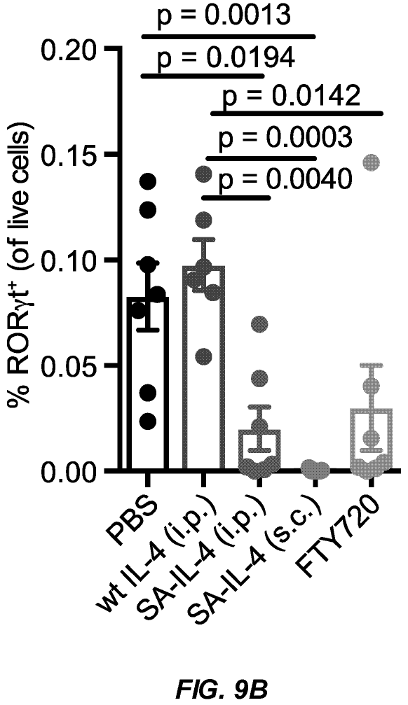

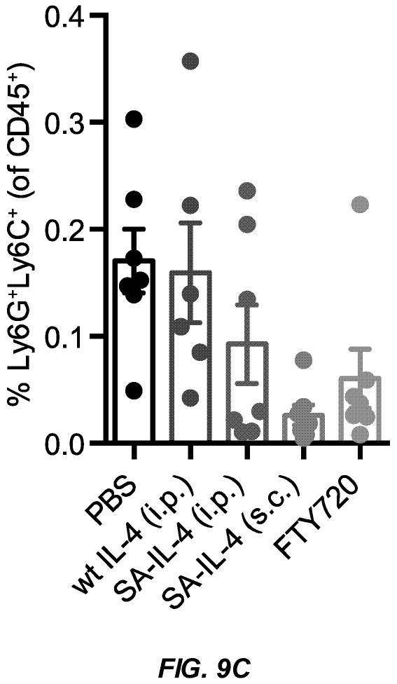

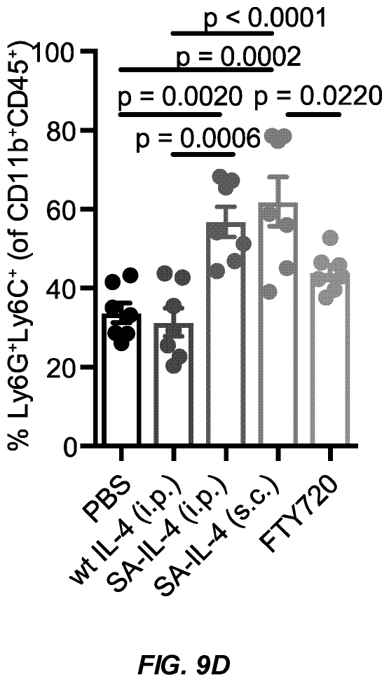

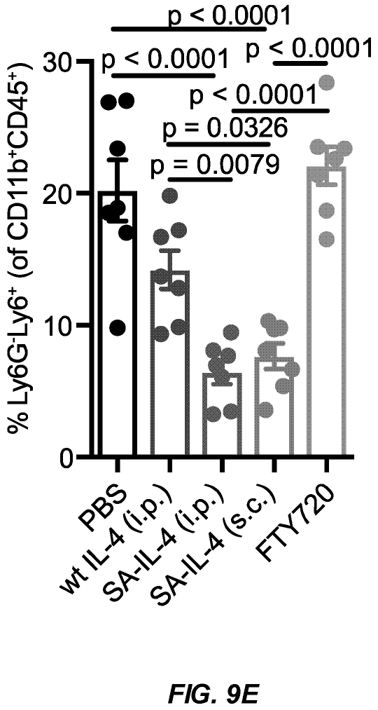

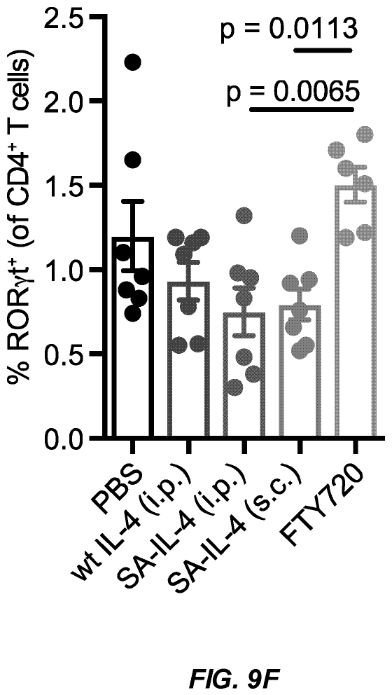

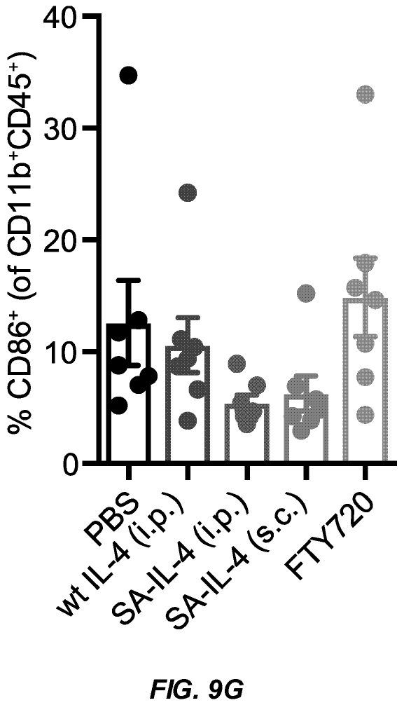

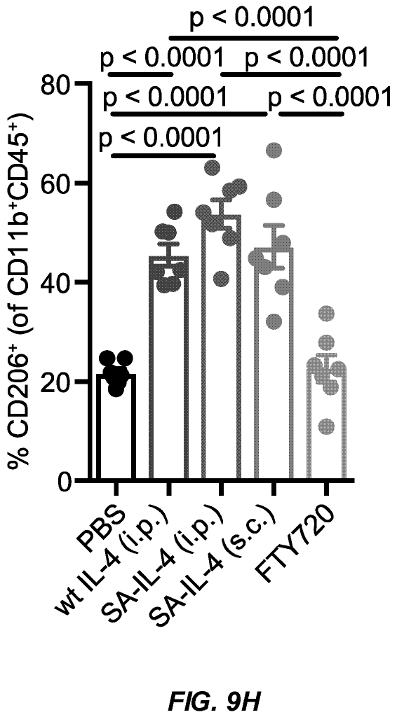

Next, immune cells in the spinal cord and spinal cord-draining lymph node (dLN) were analyzed after treatment. Strikingly, s.c. injection of SA-IL-4 significantly suppressed immune cell infiltration into the CNS; very few CD45⁺ immune cells were detected within the spinal cord (FIG. 9A). Hence, Th17 (RORγt+ cells) cells in the spinal cord were barely detectable in the s.c. SA-IL4 treatment group (FIG. 9B). I.p. injection of SA-IL-4 suppressed immune cell infiltration in 4 of 7 mice, which corresponded to the incidence of EAE disease in that group. FTY720 administration also suppressed immune cell infiltration into the spinal cord, as expected. Wt IL-4 did not show any effects on immune cell infiltration, including Th17, into the spinal cord compared to PBS treatment.

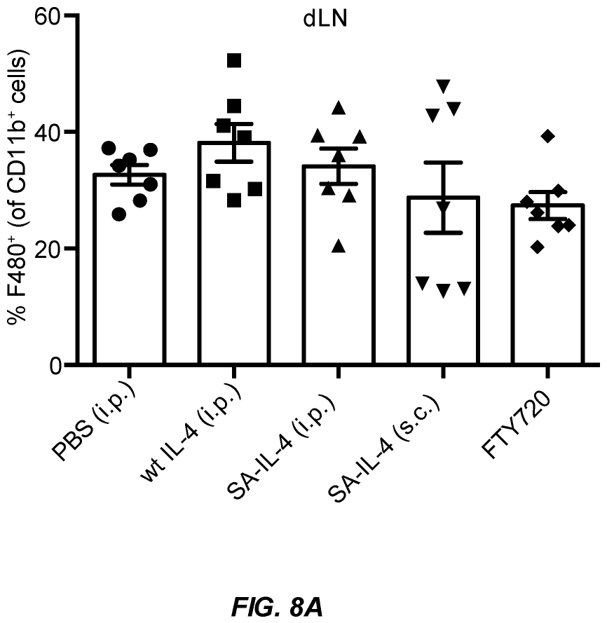

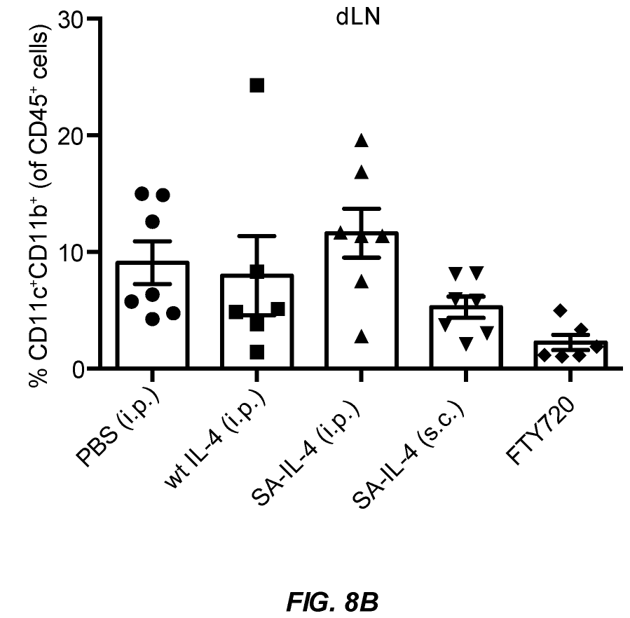

The inventors then analyzed immune cells in the lumbar dLN. SA-IL-4 increased granulocyte-like myeloid-derived suppressor cells (G-MDSCs), but reduced monocyte-like MDSCs (M-MDSCs) (FIGS. 9C and 9D). The frequency of Th17 cells within CD4⁺ T cells in the dLN was also reduced by SA-IL-4 treatment (both i.p. and s.c.), compared to FTY720 treatment (FIG. 9E). FTY720 treatment trended to increase Th17 cell frequency in the dLN compared to PBS group, probably because FTY720 inhibits lymphocyte egress from LNs. SA-IL-4 treatment reduced the frequency of M1 macrophages and increased M2 macrophages in the dLN (FIG. 9F). Wt IL-4 did not decrease the frequency of M1 macrophages but increased M2 macrophages. The frequency of macrophages within CD11b⁺ cells was maintained (FIG. 8A), as well as the frequency of DCs within CD45⁺ cells (FIG. 8B). B cells reportedly promote induction of EAE by facilitating reactivation of T cells¹⁷. SA-IL-4 (s.c.) decreased the frequency of B cells compared to both PBS and FTY720 treatment groups (FIG. 9H). Taken together, these data demonstrate that SA-IL-4 treatment generates an immunosuppressive environment in dLN and prevents immune cell infiltration into the spinal cord.

5. SA-IL-4 Treatment Decreased IL-17-Related Cytokine and Integrin Expression on Antigen-Reacting CD4⁺ T Cells

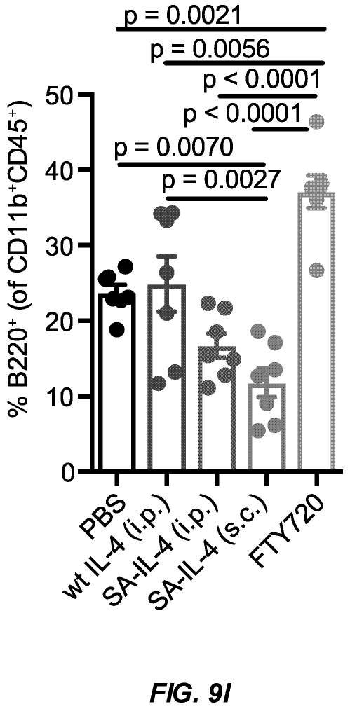

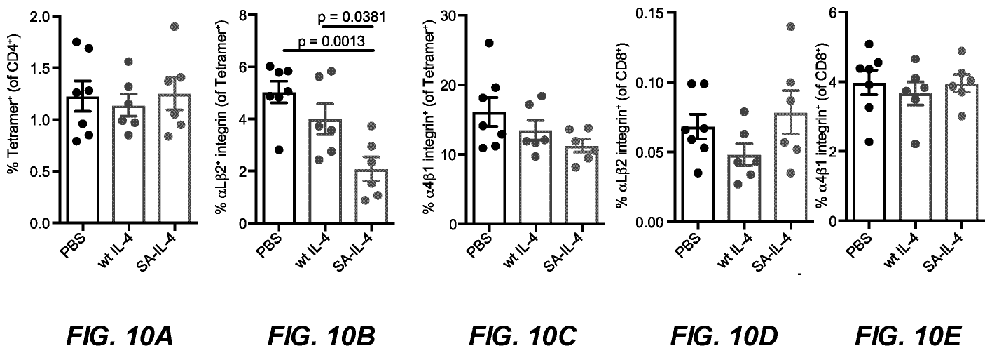

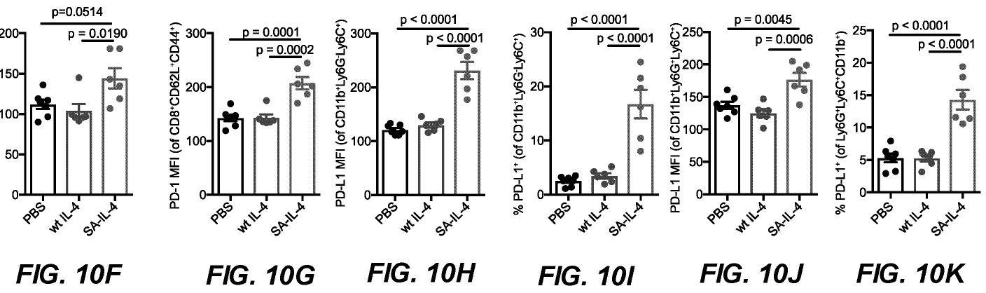

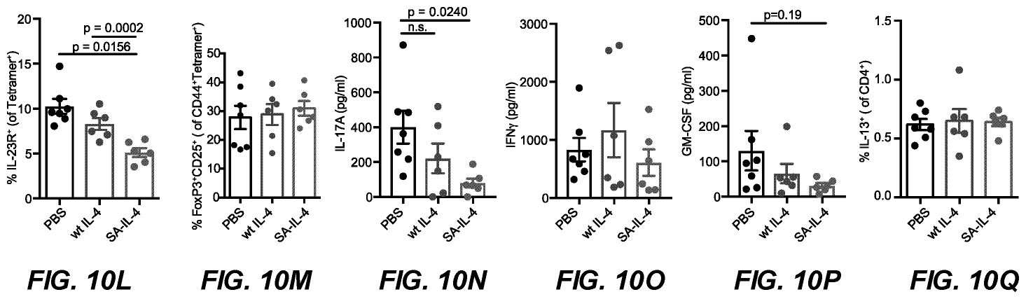

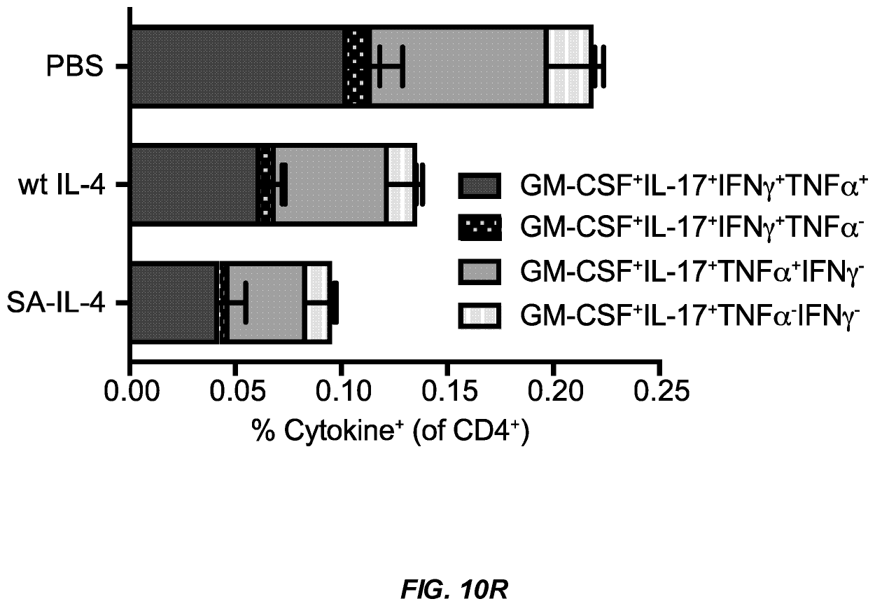

The inventors next analyzed the molecular mechanisms of decreased immune cell infiltration in the spinal cord and of the complete EAE disease prevention by s.c. injection of SA-IL-4. It was found that the number of MOG₃₅₋₅₅-reactive T cells in the dLN was maintained in all treatment groups, suggesting that SA-IL-4 does not change antigen recognition (FIG. 10A). Thus, it was hypothesized that SA-IL-4 changes T cell functionality. First, the migratory ability of T cells was tested. Expression levels of αLβ2 (LFA-1) and α4β1 (VLA-4) integrins, crucial adhesion molecules for lymphocyte migration¹⁸, are reportedly decreased by IL-4¹⁹. It was found that SA-IL-4 treatment significantly decreased αLβ2 integrin expression on MOG₃₅₋₅₅-reactive CD4⁺ T cells, but not on total CD8⁺ T cells (FIGS. 10B-10E). Expression of α4β1 integrin was not significantly changed on MOG₃₅₋₅₅-reactive CD4⁺ T cells or on total CD8⁺ T cells. Because αLβ2 integrin is indispensable for Th17 cell infiltration into the spinal cord¹⁸, this suggests that down-regulation of integrin expression is one of the mechanisms by which SA-IL-4 decreases lymphocyte migration to the spinal cord.

The inventors then tested PD-1 expression on T cells and PD-L1 expression on MDSCs (FIG. 10F-10K), as PD-1/PD-L1 interactions suppress T cell activation²⁰. Strikingly, SA-IL-4 but not wt IL-4 increased the expression of PD-1 on both central memory CD4⁺ T cells and central memory CD8⁺ T cells (FIGS. 10F and 10G). Moreover, SA-IL-4 but not wt IL-4 increased the expression levels of PD-L1 and the frequency of PD-L1-expressing cells on both M-MDSCs and G-MDSCs (FIG. 10H-10K). These data suggested T cell suppression may be induced through MDSCs and the PD-1/PD-L1 axis.

The inventors next analyzed Th17-related protein expression. IL-23 is a crucial cytokine for Th17 functionality. IL-4 reportedly binds to APCs and silences IL-23 and concordant Th17 differentiation²¹. It was found that SA-IL-4 treatment decreases the frequency of IL-23R⁺ cells within the MOG₃₅₋₅₅-reactive T cell repertoire (FIG. 10L). SA-IL-4 did not affect the frequency of Tregs (FIG. 10M).

Next, the inventors re-stimulated splenocytes harvested at day 13 with MOG protein (FIG. 10N-10P). ELISA of culture supernatant revealed a decrease in IL-17A expression with SA-IL-4 treatment, but not wt IL-4 treatment, compared to PBS (FIG. 10N). The reduction in IL-17 expression implies a decreased number and/or level of activity of MOG₃₅₋₅₅-reactive Th17 cells in the SA-IL-4 treated group. The IFNγ concentration was maintained, suggesting little effect of SA-IL-4 on Th1 cells (FIG. 10O). SA-IL-4 trended toward a decreased level of GM-CSF, a reportedly pathogenic cytokine for EAE²² (FIG. 10P). The inventors then tested cytokine expression within T cells by flow cytometry after MOG₃₅₋₅₅ peptide re-stimulation of splenocytes (FIGS. 10Q and 10R). IL-13 was analyzed, to determine if the IL-4 treatment was skewing cells towards a Th2 lineage, as well as the pathogenic cytokines associated with EAE, namely GM-CSF, IL-17, IFNγ and TNFα. SA-IL-4 did not increase IL-13 production upon restimulation, suggesting that there was not skewing towards Th2 (FIG. 10Q). SA-IL-4 decreased the frequency of cytokine-expressing cells within the CD4⁺ T cell compartment, compared to other treatments. These results strongly suggest that Th17 cells in the SA-IL-4-treated mice are fewer and less pathogenic compared to other treatment groups (FIG. 10R). Taken together, these data indicate that SA-IL-4 modulated multiple immune cell responses in the SLOs and suppressed EAE disease development through preventing immune cell infiltration, especially T cell infiltration, into the spinal cord.

6. SA-IL-4 Recovers Paralysis Due to EAE in the Chronic Phase

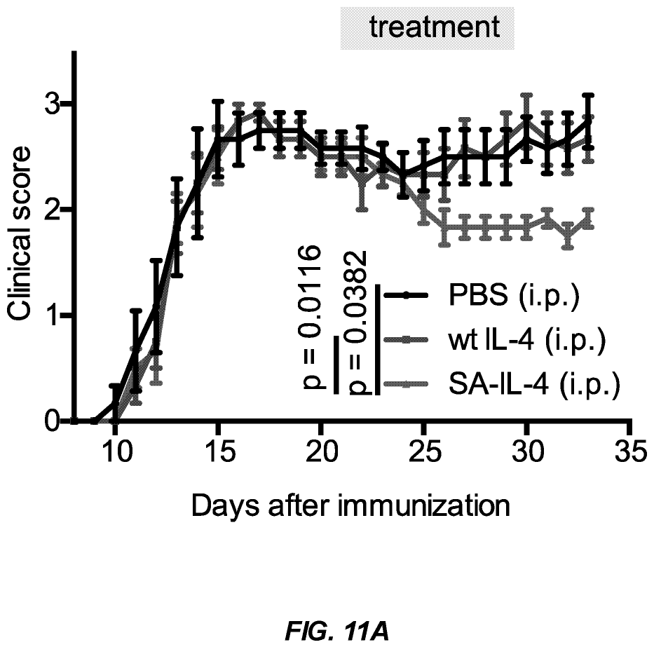

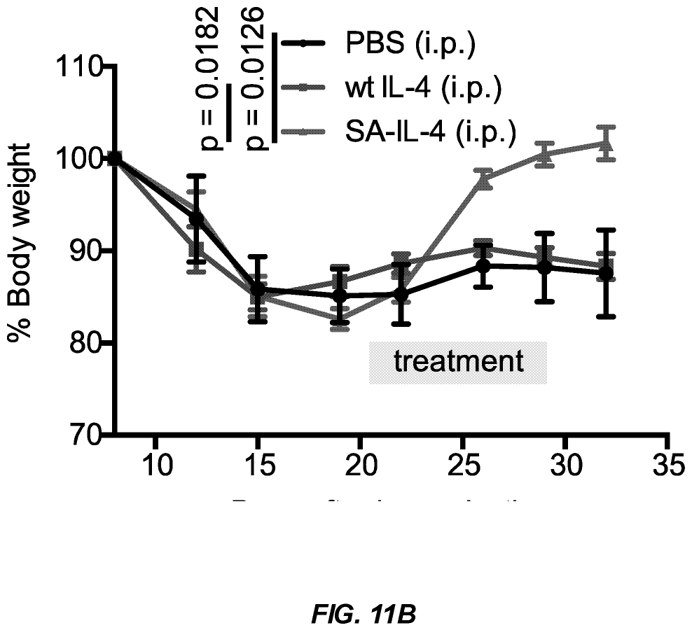

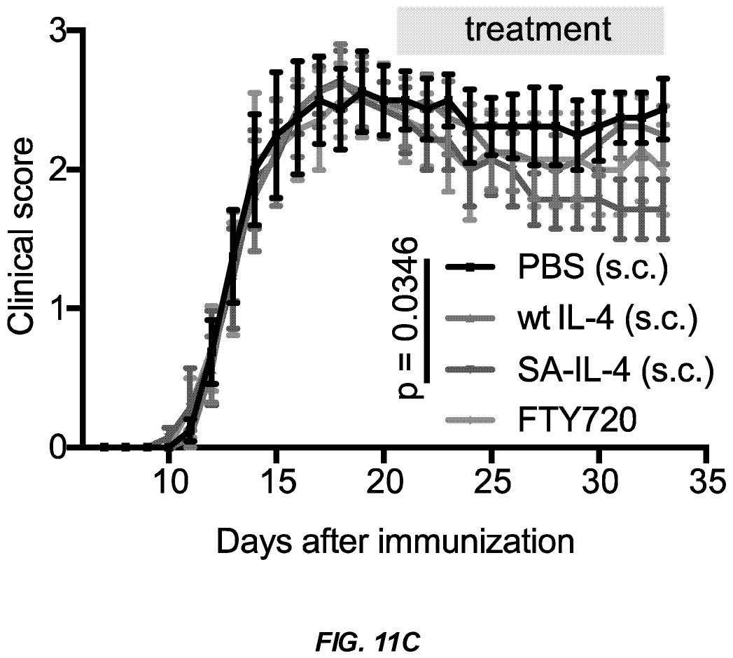

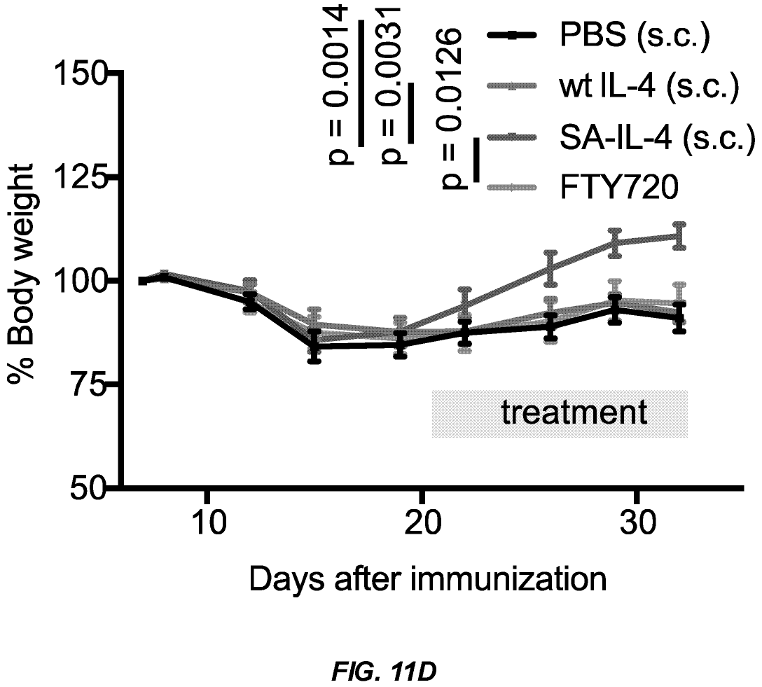

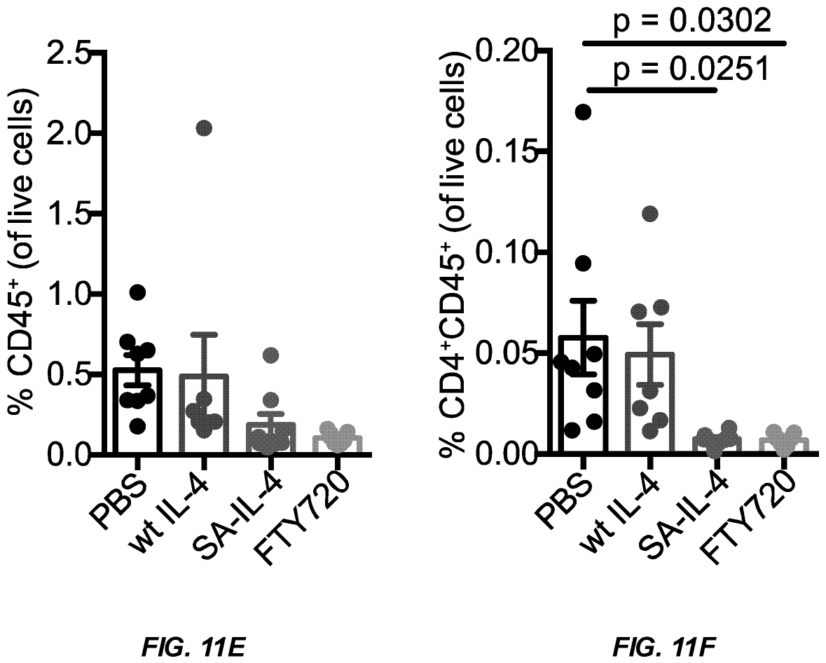

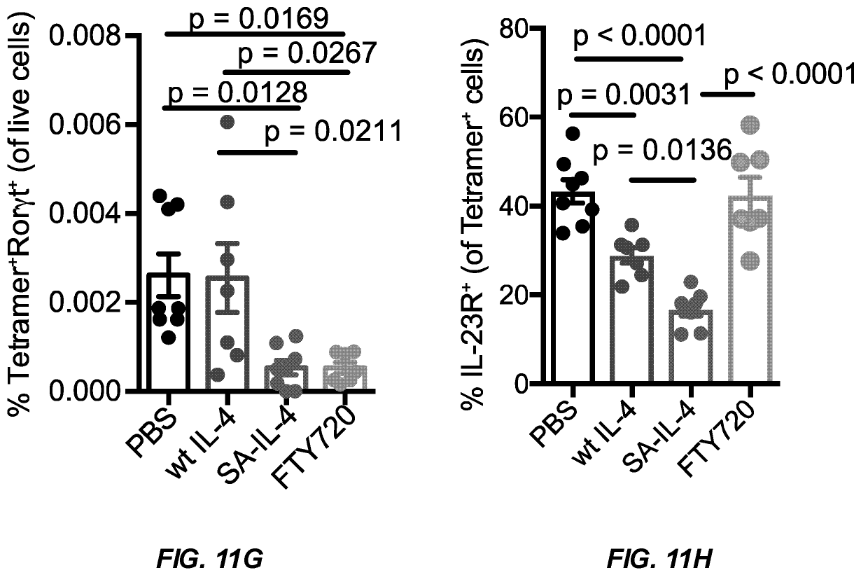

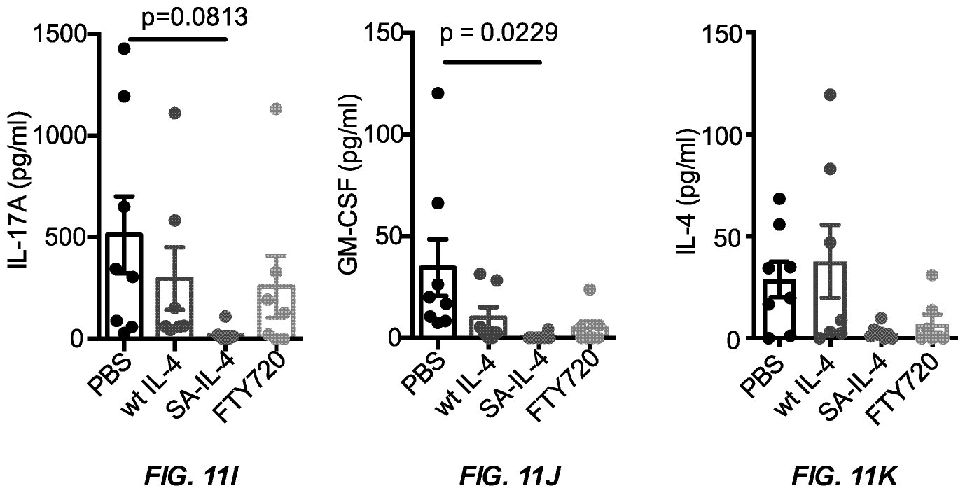

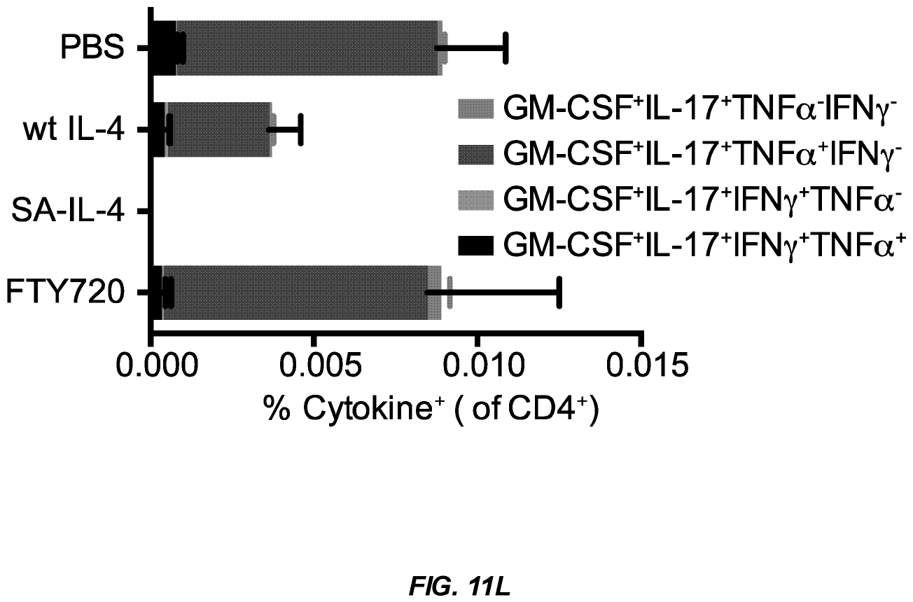

To determine if SA-IL-4 had a therapeutic effect in the chronic phase of EAE, the inventors designed an experiment involving treating mice that had already reached a stage of severe paralysis. I.p. injection of IL-4 was initiated at day 21 after induction (FIGS. 11A and 11B). Strikingly, SA-IL-4 but not wt IL-4 demonstrated therapeutic efficacy even when administered at this late time point. SA-IL-4—but not wt IL-4-treated mice gained weight, indicating disease recovery. The inventors next tested the effect of SA-IL-4 in the chronic phase via s.c. injection, and compared with oral FTY720 therapy (FIGS. 11C and 11D). SA-IL-4-treated mice had toward a decrease of the clinical score compared to the PBS treatment group. Mice receiving SA-IL-4 gained weight compared to other groups. FTY720- and wt IL-4-treated mice did not gain weight compared to PBS-treated mice.

The inventors then tested immune cell infiltration into the spinal cord at day 34 after induction by flow cytometry (FIG. 11E-11G). SA-IL-4 and FTY720 treatment decreased the number of spinal cord infiltrated immune cells, including CD4⁺ T cells and MOG₃₅₋₅₅-reactive Th17 cells, compared to PBS and wt IL-4 treatment. SA-IL-4 decreased IL-23R expressing cells within MOG₃₅₋₅₅-reactive CD4⁺ T cells in the spleen compared to other treatment groups (FIG. 11H). Finally, splenocyte re-stimulation with MOG protein was performed. ELISA of the culture supernatant revealed decreased IL-17A and GM-CSF concentrations in the SA-IL-4 treatment group, but not after wt IL-4 and FTY720 treatment, compared to PBS (FIGS. 11I and 11J). IL-4 expression was not changed by SA-IL-4 treatment (FIG. 11K), confirming a lack of Th2 skewing. Flow cytometric analysis after MOG₃₅₋₅₅ peptide re-stimulation showed that SA-IL-4 decreased the frequency of cytokine-expressing cells within the CD4⁺ T cell compartment compared to other treatments (FIG. 11L). Taken together, these results indicate that SA-IL-4 treatment has a potent therapeutic effect on the EAE chronic phase.

7. SA-IL-4 Did not Show Marked Toxicity after Systemic Injection

To test if SA-IL-4 exhibits any adverse effects, the inventors analyzed serum using a biochemistry analyzer and blood using a hematology analyzer. SA-IL-4 treatment did not increase organ damage markers nor change blood cell counts. SA-TL-4 and wt IL-4 induced splenomegaly. Wt IL-4 induced pulmonary edema, indicated by water content increase in the lung, whereas SA-IL-4 did not. These data suggest that SA-IL-4 is safe after systemic administration.

8. SA-IL-33 and SA-IL-4 Prevent EAE Disease Progression and Development

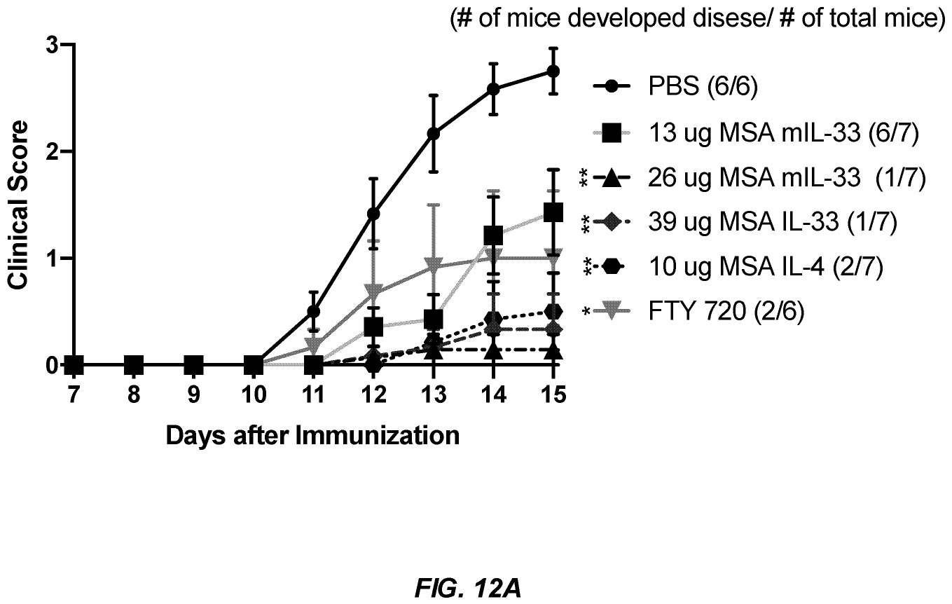

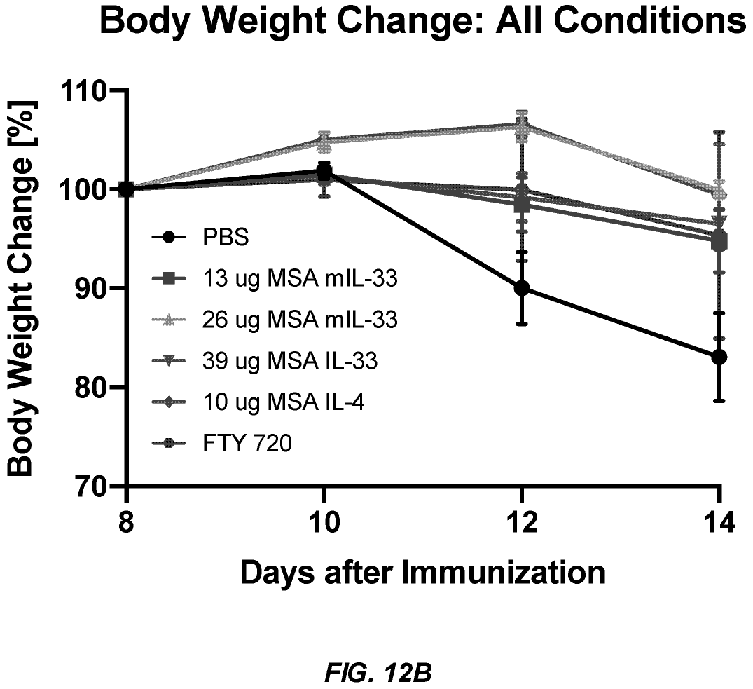

Myelin oligodendrocyte glycoprotein (MOG)35-55 experimental autoimmune encephalomyelitis (EAE) mice were injected every other day for 10 days from day 8 after immunization with phosphate-buffered saline (PBS) subcutaneously (s.c.), SA-IL-33 (13-39 g, based on IL-33, as shown in FIG. 12A) subcutaneous, or SA-IL-4 (10 μg, IL-4 base) or dosed with FTY720 1 mg/kg daily by oral administration. n=6-7 per group. FIG. 12A shows the clinical score progression for all groups. SA-IL-33 and SA-IL-4 treatment significantly reduced disease progression and severity compared with the PBS-treated group. FIG. 12B shows body weight progression for all groups.

B. Materials and Methods

1. Production and Purification of Recombinant Proteins

The sequences encoding for mouse SA without pro-peptide (25 to 608 amino acids of whole serum albumin), mouse IL-4, and a (GGGS)₂ linker were synthesized (SEQ ID NO:X6) and subcloned into the mammalian expression vector pcDNA3.1(+) by Genscript. A sequence encoding for 6 His was added at the C-terminus for further purification of the recombinant protein. The amino acid sequence of the protein is shown in Table 1. Suspension-adapted HEK-293F cells were routinely maintained in serum-free FreeStyle 293 Expression Medium (Gibco). On the day of transfection, cells were inoculated into fresh medium at a density of 1×10⁶ cells/ml. 2 μg/ml plasmid DNA, 2 μg/ml linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO₂. Seven days after transfection, the cell culture medium was collected by centrifugation and filtered through a 0.22 μm filter. Culture media was loaded into a HisTrap HP 5 ml column (GE Healthcare), using an ÄKTA pure 25 (GE Healthcare). After washing the column with wash buffer (20 mM NaH₂PO₄, 0.5 M NaCl, pH 8.0), protein was eluted with a gradient of 500 mM imidazole (in 20 mM NaH₂PO₄, 0.5 M NaCl, pH 8.0). The protein was further purified by size exclusion chromatography using a HiLoad Superdex 200PG column (GE Healthcare) using PBS as an eluent. All purification steps were carried out at 4° C. The expressed proteins were verified as >90% pure by SDS-PAGE. Purified proteins were tested for endotoxin via the HEK-Blue TLR4 reporter cell line, and endotoxin levels were confirmed to be less than 0.01 EU/ml. Protein concentration was determined through absorbance at 280 nm using NanoDrop (Thermo Scientific).

2. Mice

C57BL/6 female mice at 8 weeks of age mice were obtained from the Charles River Laboratories. Mice were housed at the University of Chicago Animal facility for at least 1 week before immunization. All experiments were performed with approval from the Institutional Animal Care and Use Committee of the University of Chicago.

3. Binding of the Proteins to Splenocytes or LN-Derived Cells

Single-cell suspensions were obtained by gently disrupting the spleen or popliteal lymph node through a 70-μm cell strainer. Red blood cells were lysed with ACK lysing buffer (Quality Biological) for splenocytes. Cells were counted and re-suspended in RPMI-1640 supplemented with 10% FBS and 1% penicillin/streptomycin (all from Life Technologies). 1××10⁵ cells/well were seeded in a 96 well microplate and were incubated with 2 μg/100 μl of SA, SA-IL4 for 30 min on ice. After 4-times washing by PBS, cells were further incubated with Rabbit monoclonal anti-mouse serum albumin antibody (clone EPR20195 abcam) for 20 min on ice. After 3-times washing by PBS, cells were incubated with 1 μg/ml Alexa Fluor 647-labeled anti-Rabbit IgG, anti-B220, anti-CD3, anti-CD4, anti-CD8, anti-CD11c, anti-CD45 and anti-F4/80 antibodies for 20 min on ice. Cells were analyzed by flow cytometry as described below.

4. Analysis of STAT6 Phosphorylation by Flow Cytometry

Mouse CD4⁺ T cells were purified from spleens of C57BL/6 mice using EasySep mouse CD4⁺ T cell isolation kit (Stem Cell). Purified CD4⁺ T cells (10⁶ cells/ml) were activated in six-well plates precoated with 5 μg/ml anti-CD3 antibody (clone 17A2, Bioxcell) and supplemented with soluble 2 μg/ml anti-CD28 antibody (clone 37.51, BioLegend) for 2 days. Culture medium was IMDM (Gibco) containing 10% heat-inactivated FBS, 1% Penicillin/Streptomycin and 50 μM 2-mercaptoethanol (Sigma Aldrich). After 2 days of culture, activated CD4⁺ T cells were stimulated with 50 ng/ml recombinant murine IL-2 (Peprotech) for 3 hr to induce IL-4Ra expression. After stimulation with IL-2, cells were washed and rested in fresh medium for 3 hr. Cells were then transferred into 96-well plates (50,000 cells/well). Indicated amounts of wt IL-4 or SA-IL-4 were applied to CD4⁺ T cells for 15 min at 37° C. to induce STAT6 phosphorylation. Cells were fixed immediately using BD Phosflow Lyse/Fix buffer for 10 min at 37° C. and then permeabilized with BD Phosflow Perm Buffer III for 30 min on ice. Cells were stained with Alexa Fluor 647 anti-pSTAT6 antibody (Clone J71-773.58.11, BD) recognizing phosphorylation of Tyr641. Staining was performed for 1 hr at room temperature (RT) in the dark. Cells were acquired on BD LSR and data were analyzed using FlowJo (Treestar). Mean fluorescence intensity (MFI) of pSTAT6⁺ population was plotted against cytokine concentration. Dose-response curve was fitted using Prism (v8, GraphPad).

5. Surface Plasmon Resonance (SPR)

SPR measurements were made with a Biacore X100 SPR system (GE Healthcare).

Murine FcRn recombinant protein (Acro Biosystems) was immobilized via amine coupling on a C1 chip (GE Healthcare) for ˜200 resonance units (RU) according to the manufacturer's instructions. SA-IL4 or SA (mouse SA, Sigma-Aldrich) was flowed at decreasing concentrations in the running buffer (0.01 M monobasic anhydrous sodium phosphate, pH 5.8, 0.15 M NaCl) at 30 μl/min. The sensor chip was regenerated with PBS, pH 7.4 for every cycle. Specific bindings of SA fusion proteins to FcRn were calculated by comparison to a non-functionalized channel used as a reference. Experimental results were fitted with Langmuir binding kinetics using BIAevaluation software (GE Healthcare).

6. Th17 Cell Differentiation In Vitro in Culture

Naïve CD4⁺ T cells were isolated from splenocytes using EasySep™ Mouse Naïve CD4⁺ T Cell Isolation Kit (STEMCELL Technologies) according to the manufacturer's instructions. 10⁵ cells were plated in the 96 well plate and cultured for 3 days. As Th17 induction media, 20 ng/mL rmIL-6 (peprotech), 10 ng/mL rmTGF-β (peprotech), 10 ng/mL rmIL-23 (peprotech), 5 μg/mL anti-IFNγ (BioXcell) containing IMDM with 5% FBS was used. 96 well plates were coated with 2 μg/mL anti-CD3 (clone 2C11, BioXcell) and 2 μg/mL anti-CD28 (clone 37.51, BioXcell) was added in the media. IL-17A concentrations in the culture media were measured by IL-17 Ready-Set-Go! Mouse Uncoated ELISA kit (Invitrogen) according to the manufacturer's protocol. Data were analyzed using Prism software (v6, GraphPad).

7. Plasma Pharmacokinetics of the Proteins

Wt IL-4 or SA-IL-4 (equivalent to 10 μg of IL-4) was injected intravenously into female C57BL/6 mice. Blood samples were collected in protein-low binding tubes at 1 min, 10 min, 30 min, 1 hr, 2 hr, 4 hr and 24 hr after injection. IL-4 concentrations in plasma were measured by IL-4 Ready-Set-Go! Mouse Uncoated ELISA kit (Invitrogen) according to the manufacturer's protocol.

8. Lymph Node, Spleen, Lung, Liver, and Spinal Cord Pharmacokinetics of the Proteins

Wt IL-4, SA-IL-4 or SA(P573K)-IL-4 (equivalent to 40 μg of IL-4) was injected intravenously into healthy or EAE-induced (day 16 after induction, see induction protocol below) C57BL/6 mice. Lumbar and brachial LNs, spleen, liver, lung, and spinal cord were collected after injection, and were subsequently homogenized using Lysing Matrix D and FastPrep-24 5G (MP Biomedical) for 40 s at 5000 beats/min in T-PER tissue protein extraction reagent (Thermo Scientific) with cOmplete™ proteinase inhibitor cocktail (Roche). After homogenization, samples were incubated overnight at 4° C. Samples were centrifuged (5000 g, 5 min), and the total protein concentration and IL-4 concentration were analyzed using a BCA assay kit (Thermo Fisher) and IL-4 Mouse Uncoated ELISA kit (Invitrogen), respectively. Simultaneously, cytokine levels in LN extract were measured using Mouse Uncoated ELISA kit (Invitrogen) or Ready-SET-Go! ELISA kits (eBioscience) according to the manufacturer's protocol.

9. Fluorescence Based IL-4 Detection in the LN

To make fluorescently labeled wt IL-4 and SA-IL-4, proteins were incubated with 8-fold molar excess of using DyLight 800 NHS ester (Thermo Fisher) for 1 hr at RT, and unreacted dye was removed by a Zebaspin spin column (Thermo Fisher) according to the manufacturer's instruction. 10 μg of DyLight 800-labeled wt IL-4 and SA-IL-4 with equivalent fluorescence were injected i.v. to naïve C57BL/6 mice. After 4 hr, the iliac LN was imaged with the Xenogen IVIS Imaging System 100 (Xenogen) under the following conditions: f/stop: 2; optical filter excitation 745 nm; excitation 800 nm; exposure time: 5 sec; small binning.

10. Analysis of FcRn Expression in Cells from Various Organs

C57BL/6 mice were euthanized, and the brachial, axillary and inguinal lymph nodes together with the liver and lung lobes were isolated and digested. Briefly, the lung lobes were cut into small pieces with a scissor and then digested in 5 ml DMEM (Gibco) with 5% FBS, 1 mg/ml Collagenase IV (LS004188, Worthington Biochemical), 3.3 mg/ml Collagenase D (11088866001, Sigma), 20 μg/ml DNAse I (LS006333, Worthington Biochemical) and 1.2 mM CaCl₂) for 1 hr at 37° C. on a shaker. The lymph nodes were punctured with syringe needles and digested in 750 μl DMEM (Gibco) with 5% FBS, 1 mg/ml Collagenase IV, 40 μg/ml DNAse I and 1.2 mM CaCl₂) for 30 min at 37° C. with magnetic stirring. Then 750 μl of 3.3 mg/ml Collagenase D, 40 μg/ml DNAse I and 1.2 mM CaCl₂) in DMEM with 5% FBS was added and each sample was further digested for another 15 min. Livers were cut into small pieces and digested in 5 ml DMEM with 5% FBS, 1 mg/ml Collagenase IV, 1 mg/ml Collagenase D, 40 μg/ml DNase I, 1.2 mM CaCl₂) for 1 hr at 37° C. on a shaker. After quenching the media with 5 mM EDTA, single cell suspensions were prepared using a 70 μm cell strainer (22-363-548, Fisher). Liver samples were centrifuged at 50×g for 5 min to pellet and discard hepatocytes. Finally, red blood cells were lysed with 1 ml ACK buffer for 90 s and neutralized with 10 ml DMEM media with 5% FBS. Single cell suspensions were counted after digestion and 1-2 million cells were stained. For antibodies against surface targets, the staining was done in PBS with 2% FBS, and for intracellular cells were stained for 2 hr in 0.5% saponin in PBS with 2% FBS. The following anti-mouse antibodies were used for flow cytometry: CD45 APC-Cy7 (clone 30-F11, BioLegend), CD31 BUV395 (clone 390, BD Biosciences), gp38 PE-Cy7 (clone 8.1.1, BioLegend), FcRn (R and D systems, 1:50 dilution), F4/80 PE (clone BM8, BioLegend), CD11c BV421 (clone N418, BioLegend), CD11b BV786 (clone M1/70, BD Biosciences), CD146 BV605 (clone ME-9F1, BD Biosciences). For FcRn staining, cells were stained with Alexa Fluor 647 donkey anti-goat IgG (Jackson ImmunoResearch, 1:400 dilution). Cell viability was determined using the fixable viability dye eFluor 455UV dye (65-0868-14, eBioscience).

11. Transendothelial Transport Assay

Human umbilical vein endothelial cells (HUVEC; Lonza) were maintained in EGM-2 medium (Lonza) and used until passage 9. 10⁵ cells were seeded in 6.5 mm-diameter, 0.4 μm-pore inserts (Corning) pre-coated with 50 μg/ml rat tail collagen Type I (Corning) in PBS and incubated for 3 days to obtain a confluent monolayer. Medium was changed in inserts and bottom wells to EGM-2 without growth factors and incubated for 2 hr. SA-IL-4 or SA(P573K)-IL-4 (10 μg/ml) was added in the inserts (apical side) followed by 3 hr incubation. Medium from both inserts (apical side) and bottom wells (basal side) was collected and IL-4 was measured by mouse IL-4 ELISA (R&D systems). Transendothelial transport was computed as the fraction of IL-4 transported to the basal side of the insert over the total amount of IL-4 applied on the apical side. To verify monolayer integrity, cells on insert membranes were fixed in PBS 2% PFA for 15 min, permeabilized with TBS 1% Triton for 10 min, stained with goat anti-human VE-Cadherin (R&D systems) in TBS 0.5% casein for 1 hr at RT followed by donkey anti-goat 594 (Invitrogen) secondary antibody in TBS 0.5% casein for 1 hr at RT. Membranes were mounted in Prolong Gold Antifade Reagent with DAPI (Invitrogen) and imaged with an Olympus IX2-DSU fluorescence microscope and a 60× objective. Z-projections were generated from fluorescence image stacks using ImageJ and Stack Focuser plugin.

12. Immunofluorescence

Wt IL-4 and SA-IL-4 were fluorescently labeled with DyLight 594 NHS ester (Thermo Fisher), as described above. 1 hr after i.v. injection of fluorescently-labeled IL-4 (40 μg for wt TL-4 and same fluorescent amount for SA-IL-4), mice were sacrificed. Mouse LNs were harvested and fixed in 2% PFA in PBS overnight and washed with PBS. After overnight incubations in 30% sucrose solutions, LNs were embedded in Optimum Cutting Temperature compound. Then, 5 μm cryosections were cut using a cryostat. Sections were then blocked with 2% BSA in PBS at RT and incubated with the following primary antibodies for 2 hr at RT: 10 μg/ml hamster anti-mouse CD3ε antibody (clone: 145-2C11, BioLegend) and 2.5 μg/ml rat anti-mouse PNAd (clone: MECA-79, BioLegend) antibody. After washing with PBS-T, tissues were stained for 1 hr at RT with the following fluorescently-labeled secondary antibodies were used: Alexa Fluor 647 goat anti-hamster (1:400, Jackson ImmunoResearch) and Alexa Fluor 488 donkey anti-rat (1:400, Jackson ImmunoResearch). The tissues were washed three times and then covered with ProLong gold antifade mountant with 4′,6-diamidino-2-phenylindole (DAPI; Thermo Fisher Scientific). An IX83 microscope (Olympus) was used for imaging with 10× magnification for CD3 staining, and a Leica SP8 3D Laser Scanning Confocal microscope with 20× magnification for PNAd staining. Images were processed using ImageJ software (NIH).

13. EAE Model

C57BL/6 young female mice aged 9 to 12 wk were immunized subcutaneously at the dorsal flanks with an emulsion of MOG₃₅₋₅₅ in complete Freund's adjuvant (CFA), followed by intraperitoneal administration of pertussis toxin (PTX) in PBS, first on the day of immunization and then again the following day. MOG₃₅₋₅₅/CFA Emulsion and PTX were purchased from Hooke Laboratories. Following the first immunization, the severity of EAE was monitored and clinical scores were measured daily from day 8 after immunization. The clinical scores were determined by A.I., M.N. or A.S. based on the Hooke Laboratories criteria (available on the World Wide Web at hookelabs.com/services/cro/eae/MouseEAEscoring.html), under blinding to the treatment grouping. IL-4, SA-IL-4, PBS was administered i.p. or s.c. (in the mouse left back flank; approximately 2 cm away from emulsion injection site) in 100 μl PBS, every other day. FTY720 (1 mg/kg body weight) was administered orally every day.

14. Histology of Spinal Cord

The thoracic and lumbar spines of EAE mice were harvested and cut out at the thoracolumbar junction. Tissues were fixed in 2% PFA overnight. After PBS wash, tissues were decalcified using Decalcifier II (Leica Biosystem) overnight. Then, tissues were embedded in paraffin. After paraffin embedding, blocks were cut into 5 mm sections. After deparaffinization and rehydration, tissue sections were treated with target retrieval solution (S1699, DAKO) and heated in a steamer for 20 min at temperature >95° C. Tissue sections were incubated with anti-mouse aMBP (abcam ab40390) for 1 hr incubation at RT in a humidity chamber. Following a TBS wash, the tissue sections were incubated with biotinylated anti-rat IgG (10 mg/mL, Vector laboratories) for 30 min at RT. The antigen-antibody binding was detected by Elite kit (PK-6100, Vector Laboratories) and DAB (DAKO, K3468) system. Slides were imaged by EVOS FL Auto (Life Technologies).

15. Flow Cytometry

EAE mice were treated with PBS, wt IL-4, or SA-IL-4 (equivalent to 10 μg of IL-4) every other day, starting 8 days after immunization. Thirteen, 17 or 34 days after immunization, the spinal cord, spleen, and lumbar LNs were harvested. Spinal cord tissues were digested in Dulbecco's Modified Eagle Medium (DMEM) supplemented with 2% FBS, 2 mg/ml collagenase D (Sigma-Aldrich) and 40 μg/ml DNase I (Roche) for 30 min at 37° C. Single-cell suspensions were obtained by gently disrupting through a 70-μm cell strainer. For spleen, red blood cells in blood were lysed with ACK lysing buffer (Quality Biological), followed by antibody staining for flow cytometry. Antibodies against the following molecules were used: anti-mouse CD38 (145-2C11, BD Biosciences), CD4 (RM4-5, BD Biosciences), anti-mouse CD8α (53-6.7, BD Biosciences), anti-mouse CD45 (30-F11, BD Biosciences), CD44 (IM7, BD Biosciences), CD62L (MEL-14, BD Biosciences), F4/80 (T45-2342, BD Biosciences), CD86 (GL1, BD Biosciences), CD206 (C068C2, BioLegend), Ly6G (1A8, BioLegend), Ly6C (HK1.4, BioLegend), CD11b (M1/70, BioLegend), CD11c (HL3, BD Biosciences), B220 (RA3-6B2, BioLegend), PD-1 (29F.1A12, BD Biosciences), PD-L1 (MIH7, BioLegend), IL-23R (078-1208, BD Biosciences), integrin aL (HI111, BD Biosciences), integrin 32 (M18/2, BD Biosciences), integrin β1 (HMb1-1, BD Biosciences), integrin α4 (R1-2, BD Biosciences), GM-CSF (MP1-22E9, BD Biosciences), IL-13 (17-7222-80, eBioscience), IL-17 (TC11-18H10.1, BD Biosciences), IFNγ (XMG1.2, BD Biosciences), TNFα (eBioscience, MP6-XT22), and FoxP3 (MF23, BD Biosciences), RoRγt antibody (Q31-378, BD Biosciences). For detection of MOG-recognizing T cells, T-Select I-Ab MOG₃₅₋₅₅ Tetramer-PE (MBL International Corporation) or MOG₃₈₋₄₉ Tetramer-PE (NIH Tetramer Core Facility) was used. Non-specific binding of MOG₃₈₋₄₉ Tetramer was not considered. Fixable live/dead cell discrimination was performed using Fixable Viability Dye eFluor 455 (eBioscience), Live/Dead Fixable Violet (eBioscience), or Live/Dead Fixable Aqua (eBioscience), according to the manufacturer's instructions. Staining was carried out on ice for 20 min. For intracellular staining, Cytofix/Cytoperm (BD Bioscience) was used to fix cells for 20 min at 4° C. For permeabilization, perm/wash buffer (BD Bioscience) was used, and cells were stained in perm/wash buffer for 30 min at 4° C. Following a washing step, cells were stained with specific antibodies for 20 min on ice prior to fixation. All flow cytometric analyses were done using a Fortessa (BD Biosciences) flow cytometer and analyzed using FlowJo software (Tree Star).

16. Re-Stimulation of Splenocytes

Single cell suspensions were created from the dLNs and spleen. For analysis of cytokine production, 5×10⁵ lymphocytes and 2×10⁶ splenocytes were plated in a 96 well round bottom plate. Cells were stimulated with 10 μM MOG₃₅₋₅₅ peptide (Genscript). After 2 hr, GolgiPlug (brefeldin A) and GolgiStop (Monensin) were added per manufacturer's protocol to block secretion of intracellular cytokines. Four hours after addition of GolgiPlug and GolgiStop, cells were stained for flow cytometry. For fixation, Cytofix/Cytoperm (BD Bioscience) was used for 20 min at 4° C. For permeabilization, perm/wash buffer (BD Bioscience) was used, and cells were stained in perm/wash buffer for 30 min at 4° C. For 3 day restimulation, 2.5×10⁵ lymphocytes or 1×10⁶ splenocytes were plated in a 96 well round bottom plate. Cells were stimulated with 10 μM MOG₃₅₋₅₅ (for 6 hr culture followed by flow cytometry) or 100 μg/ml MOG protein (for 72 hr culture) (Anaspec). After 72 hr, supernatant was collected for analysis by ELISA using Ready-Set-Go! Kit (Invitrogen) or LEGEND MAX mouse GM-CSF ELISA kit (BioLegend).

17. Safety Assessments of SA-IL-4

C57BL/6 mice were intravenously injected with PBS, wt IL-4, or SA-IL4 (equivalent to 10 μg of IL-4). Two days after, blood samples collected from mice were analyzed using a COULTER Ac⋅T 5diff CP hematology analyzer (Beckman Coulter) according to the manufacturer's instructions. Lung and spleen were harvested and weighed. Water content in the lung was determined by weighing before and after overnight lyophilization using a FreeZone 6 Benchtop Freeze Dryer (Labconco). Serum samples collected from PBS, wt IL-4, and SA-IL-4-injected mice were analyzed using a Biochemistry Analyzer (Alfa Wassermann Diagnostic Technologies) according to the manufacturer's instructions.

18. Statistical Analysis

Statistically significant differences between experimental groups were determined using Prism software (v6, GraphPad). Where one-way ANOVA followed by Tukey's HSD post hoc test was used, variance between groups was found to be similar by the Brown-Forsythe test. For single comparisons, a two-tailed Student's t-test was used. P values less than 0.05 are considered significantly different.

### Example 2—Enhanced Lymph Node Trafficking of Engineered IL-10 Suppresses Rheumatoid Arthritis in Murine Models

A. Results

1. Albumin-Fused IL-10 Binds to Neonatal Fc Receptor (FcRn) and APCs and Accumulates within the LNs

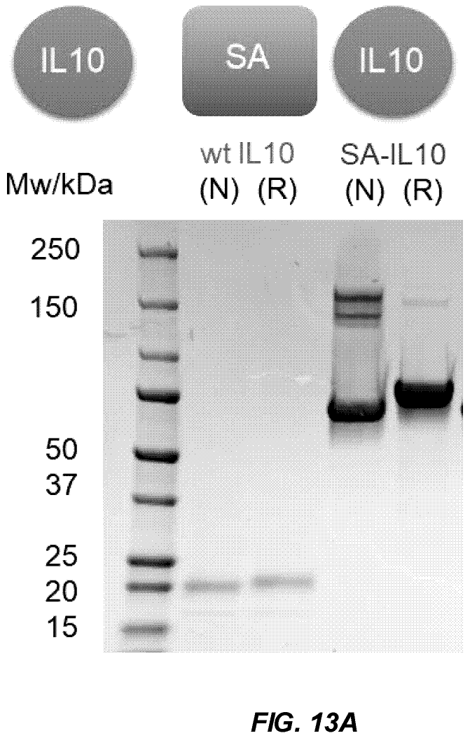

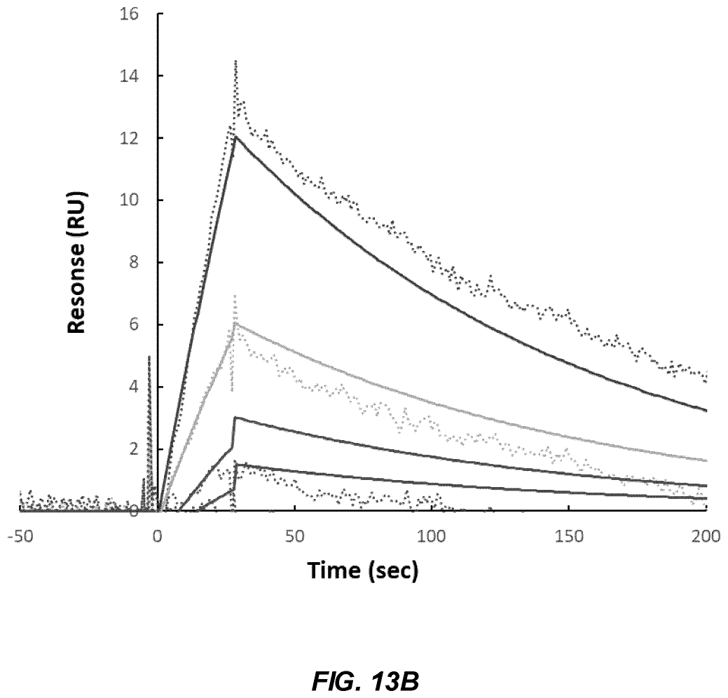

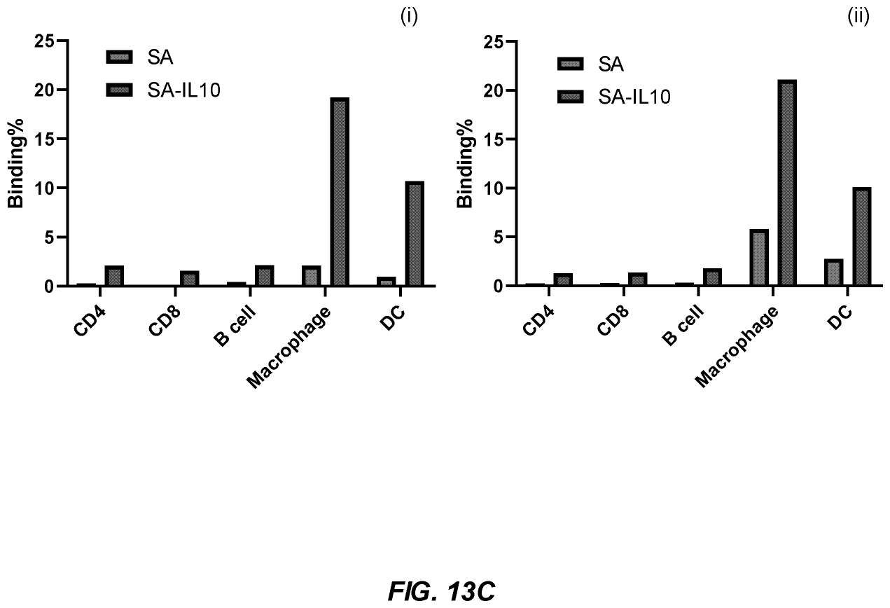

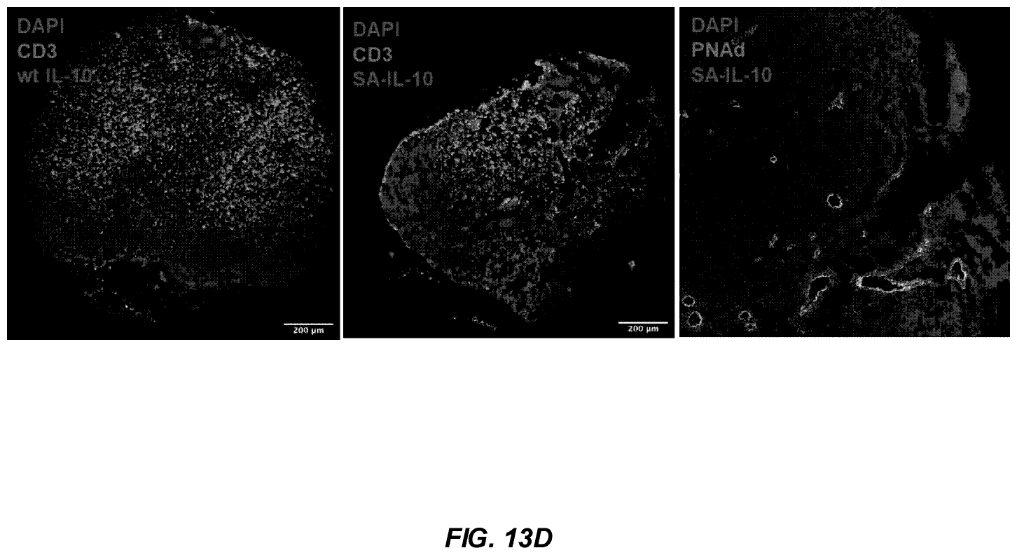

Wild type (wt) mouse IL-10, and SA-fused mouse IL-10 were recombinantly expressed, and the molecular weight of the fusion protein was correspondingly higher than for wt IL-10 as determined by SDS-PAGE; in addition, most of the SA-IL-10 existed as a monomer under non-reducing conditions (FIG. 13A). Surface plasmon resonance (SPR) analysis revealed that SA-IL-10 binds to FcRn with micromolar order of K_{d} (FIG. 13B). The binding ability of these proteins to splenocytes and single cells isolated from the popliteal LN was further evaluated by flow cytometry (FIG. 13C). SA-fused IL-10 exhibited high binding to macrophages and dendritic cells in both splenocytes and LN-derived cells. After intravenous administration of fluorescently-labeled SA-IL-10, significantly higher fluorescence signals were observed within the popliteal LN compared with wt IL-10 (FIG. 13D). Interestingly, higher fluorescence signals were located surrounding high endothelial venules (HEVs), where antigen presenting cells (APCs) reside (43).

2. Albumin-Fused IL-10 Shows Prolonged Blood Circulation

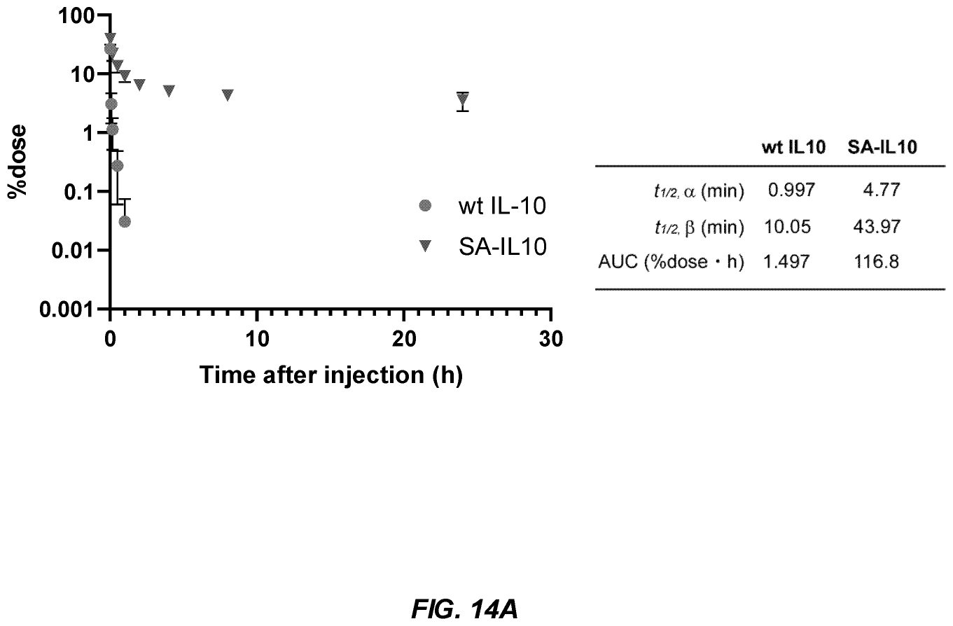

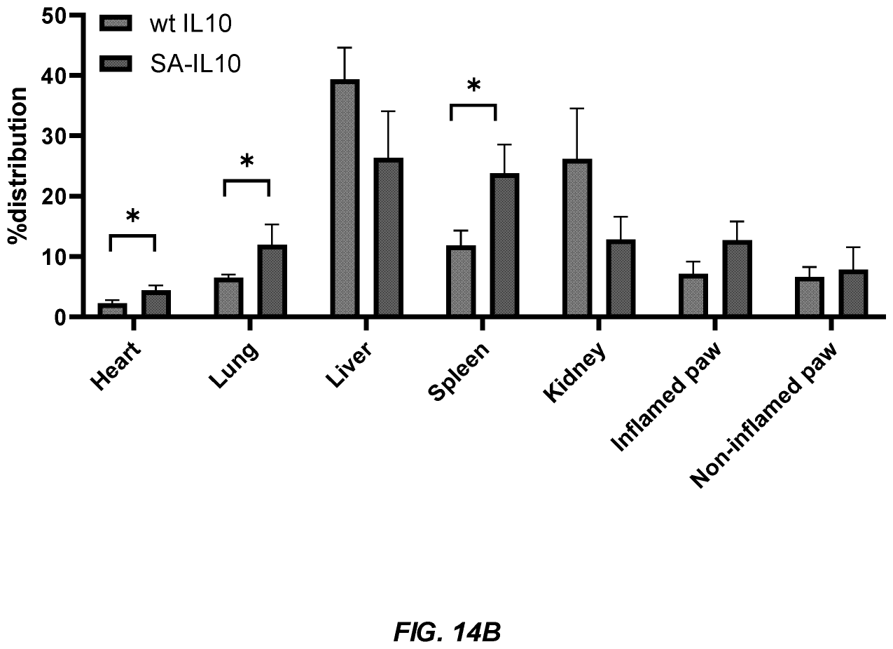

SA is known to demonstrate long circulation via FcRn-mediated recycling on vascular endothelial cells (44,45). SA-IL-10 showed significantly prolonged blood circulation compared with wt IL-10 (FIG. 14A). FIG. 14B shows the fluorescence signals from major organs of mice intravenously injected with DyLight800-labeled proteins. Reflecting its long circulation properties, SA-IL-10 showed higher signals in the heart, lungs and spleen than that of wt IL-10.

3. Albumin-Fused IL-10 Reduces Immune Activity after Accumulation within LNs

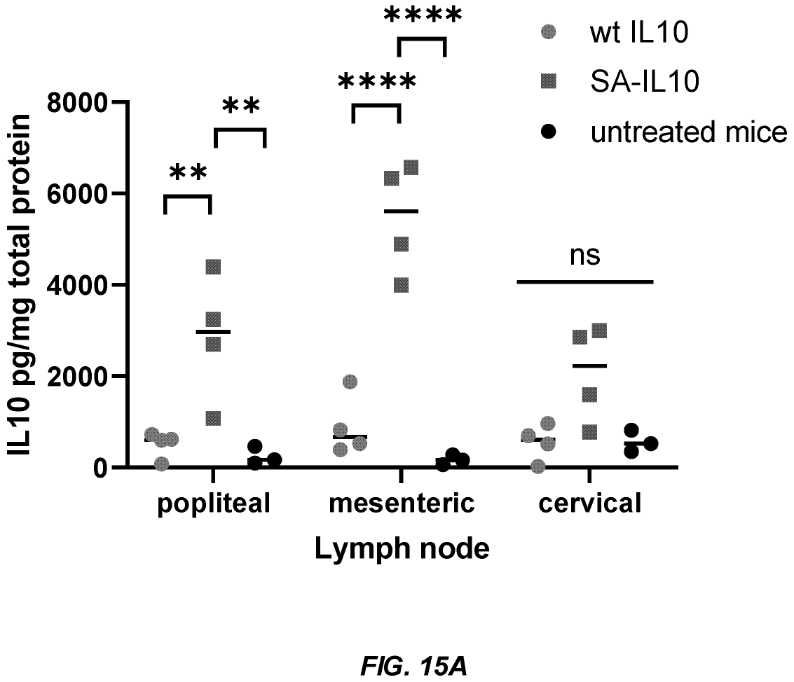

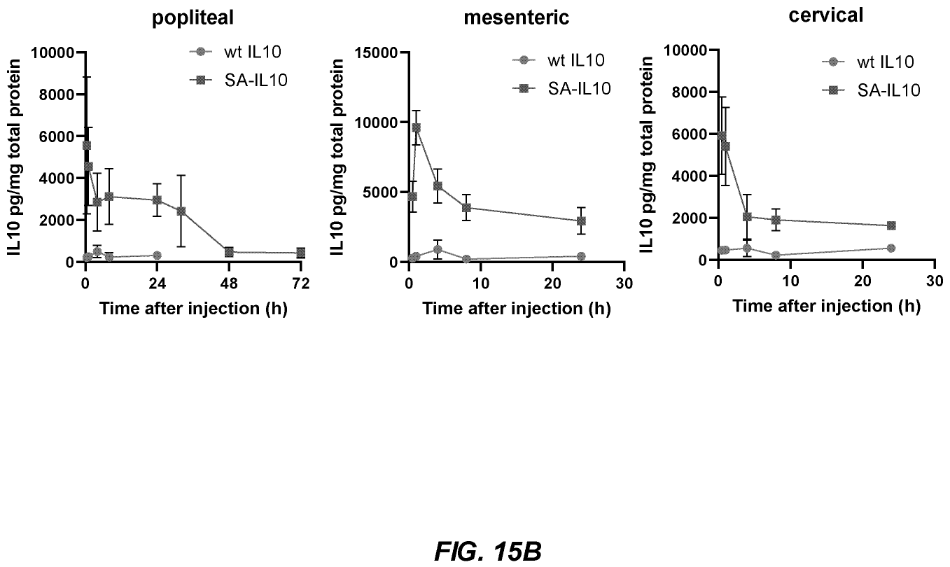

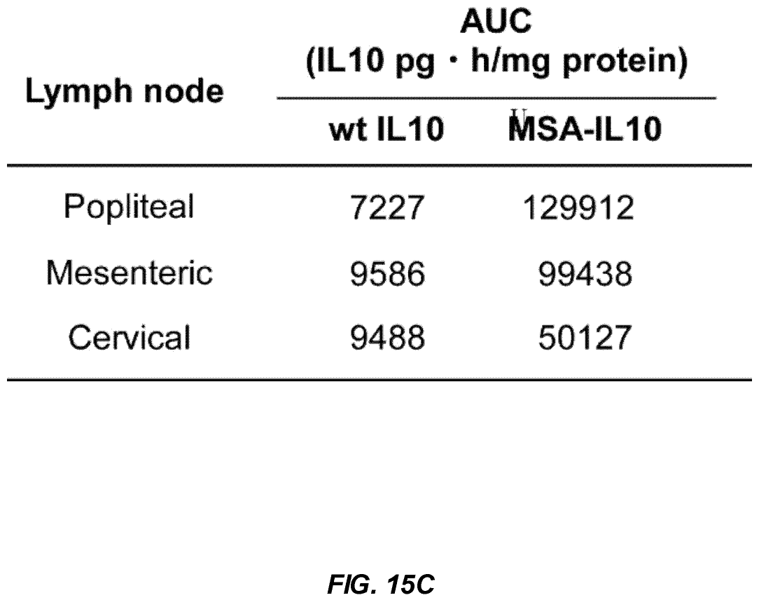

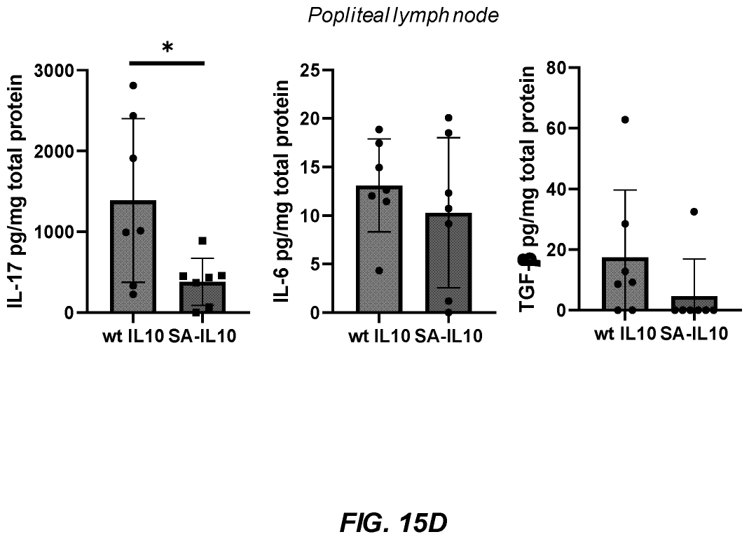

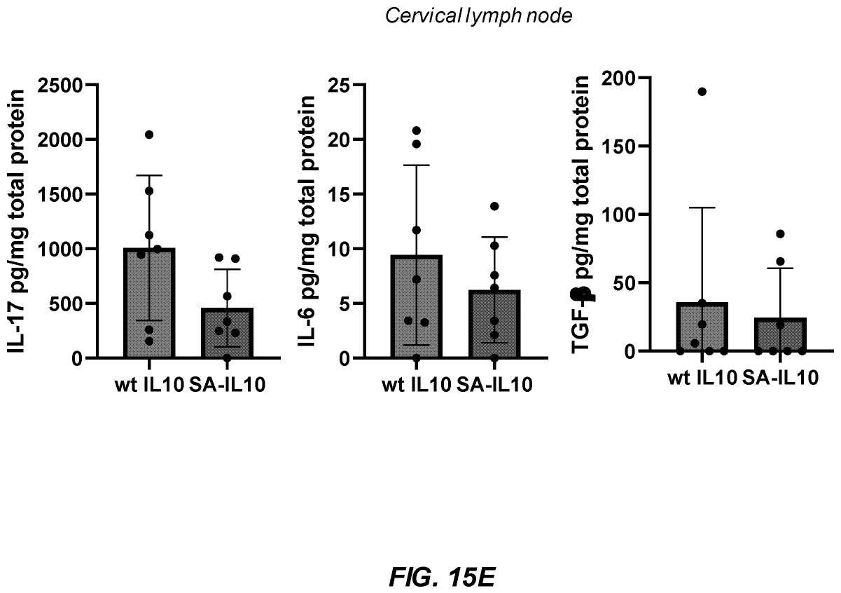

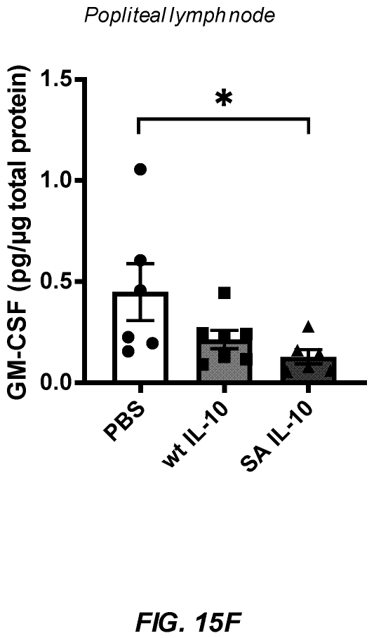

SA-fused IL-10 showed micromolar affinity to FcRn (FIG. 13B) and accumulation within LNs after intravenous injection (FIG. 13D). Next, the amounts of IL-10 and its pharmacokinetics in the LNs were quantitatively evaluated (FIG. 15A-15C). After intravenous injection of wt IL-10, or SA-IL-10 within CAIA mice, IL-10 concentrations in the LNs at various time points were detected using ELISA. SA-IL-10 showed significantly higher IL-10 signals in the joint-draining (popliteal) LN, the mesenteric LN and relatively high signals in a non-draining (cervical) LN compared with wt IL-10 at 4 hr after injection (FIG. 15A). SA-IL-10-injected mice also showed a peak for IL-10 concentration at around 1 hr after injection (FIG. 15B) and 5-10 times higher AUC than wt IL-10 in the LNs (FIG. 15C). These data indicated that SA-IL-10 immediately accumulated within LNs after intravenous injection and showed higher retention in the LNs compared with wt IL-10.

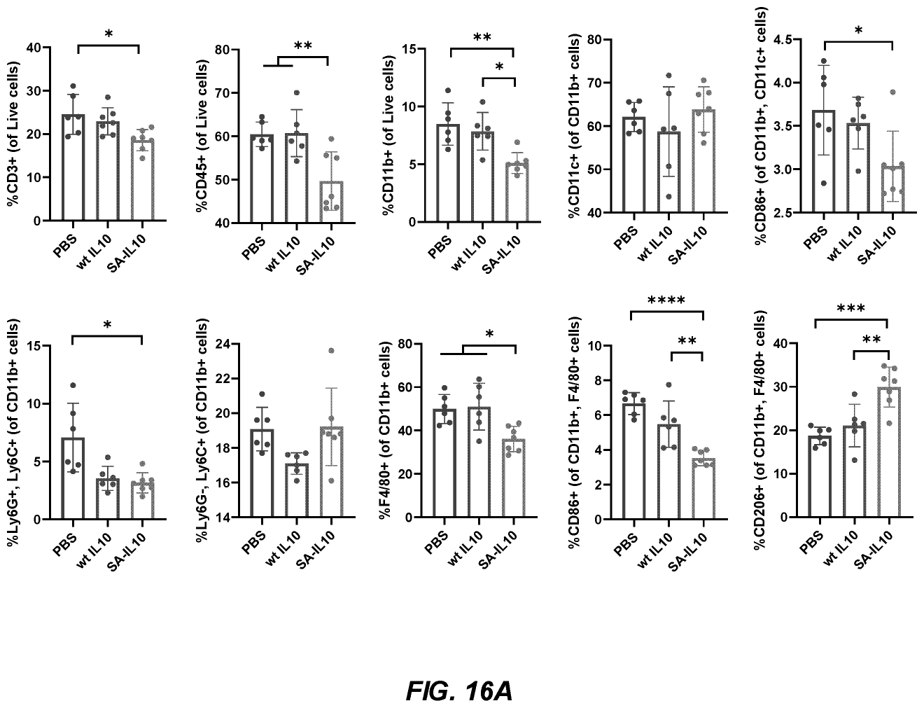

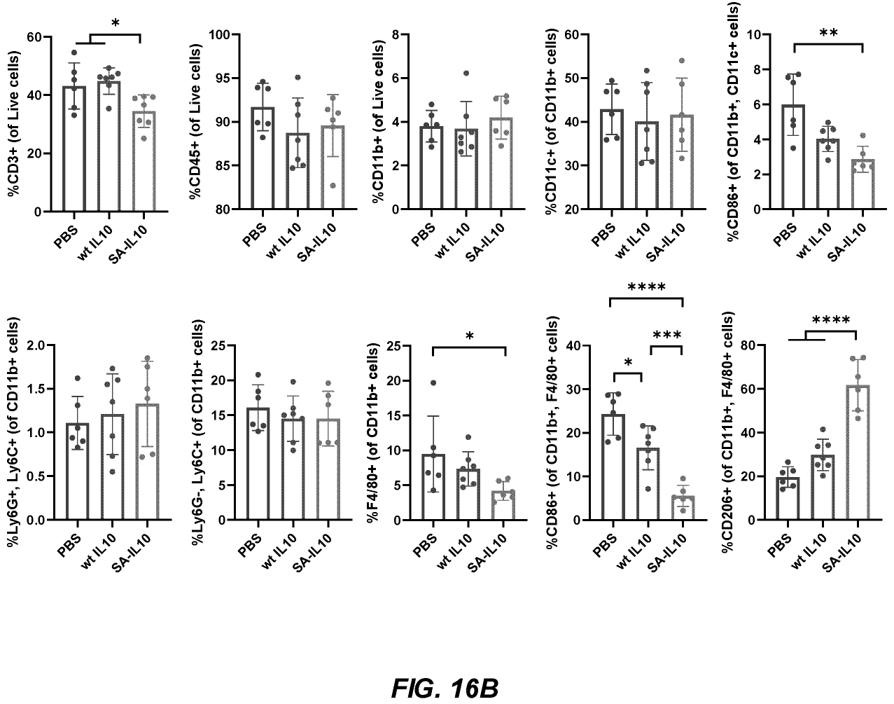

High concentrations and AUC of SA-IL-10 in the LNs may affect the phenotypes of various immune cells in LNs and other secondary lymphoid organs. Therefore, immune cell populations in the spleen and popliteal LN were analyzed by flow cytometry (FIGS. 16A-B). Intravenous injection of SA-IL-10 induced a significant decrease in the frequency of CD3⁺ T cells and CD45⁺ lymphocytes in the spleen (FIG. 16A). In addition, the frequencies of CD86⁺ dendritic cells, granulocytic myeloid-derived suppressor cells (G-MDSCs), and CD86⁺ M1 macrophages decreased and that of CD206⁺ M2 macrophages increased after injection of SA-IL-10 compared with PBS or wt IL-10. A similar tendency was observed within the popliteal LNs (FIG. 16B). These data suggested that SA-IL-10 suppressed the activity of APCs and simultaneously activated immunosuppressive M2 macrophages. Deactivation of APCs in the LNs, and high accumulation of IL-10 might suppress the activity of Th17 cells, which play a crucial role in the development of RA (46, 47). Th17-relating cytokines (IL-17, IL-6 and TGF-3) were measured in the LNs in the joint-draining (popliteal) and a non-draining (cervical) LN: compared to treatment with wt IL-10, IL-17 was statistically reduced in the popliteal LN after treatment by SA-IL-10, and levels in the cervical LN were not statistically reduced by either IL-10 variant (FIGS. 15D and 15E). Treatment by SA-IL-10 reduced the concentration of GM-CSF in the popliteal LN, whereas wt IL-10 did not (FIG. 15F).

4. Albumin-Fused IL-10 Suppresses the Development of Rheumatoid Arthritis

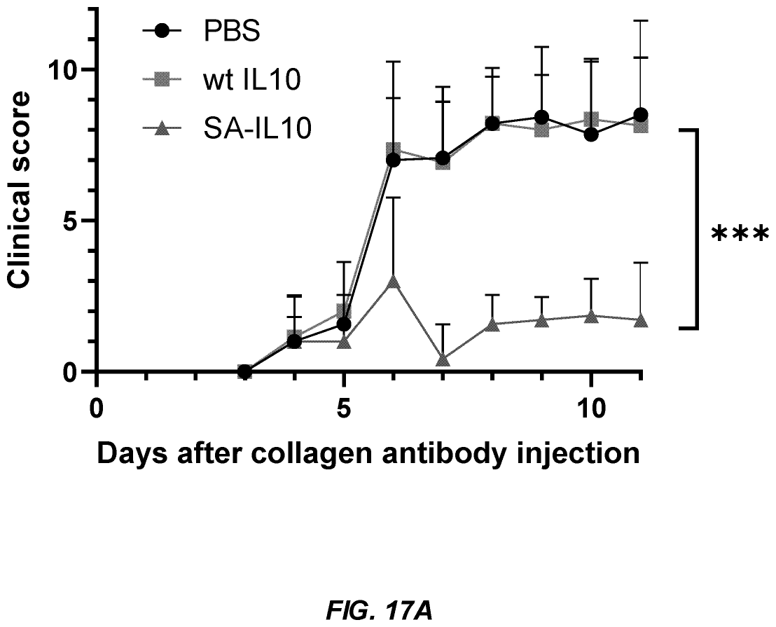

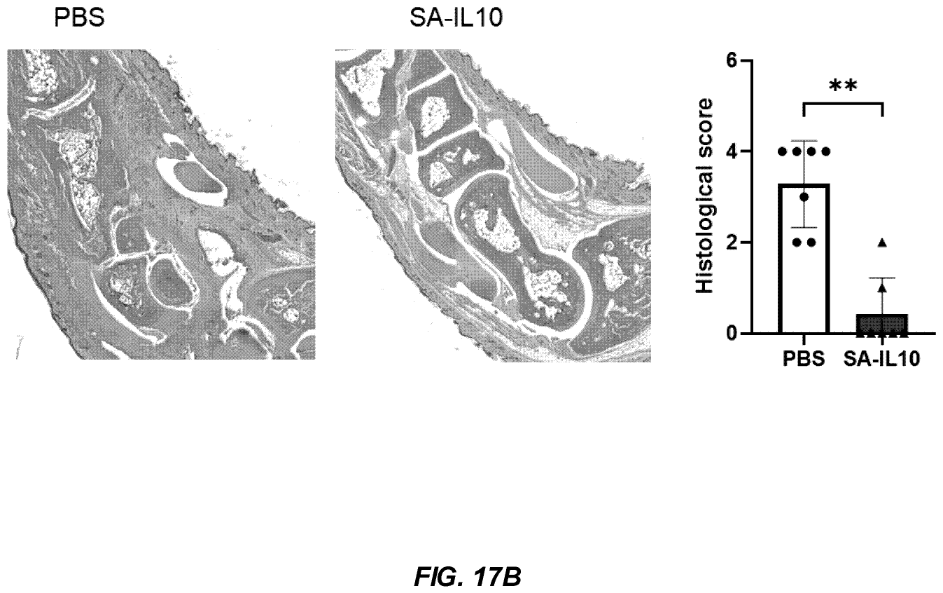

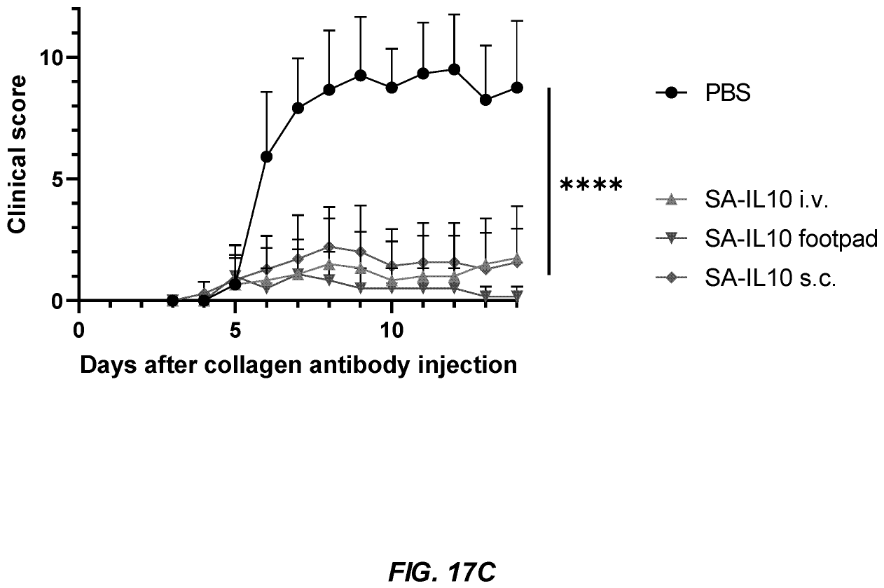

The therapeutic effects of engineered IL-10 in the passive collagen antibody-induced arthritis (CAIA) model were evaluated (FIGS. 17A-17C). Intravenous injection of SA-IL-10 significantly suppressed the development of arthritis, whereas PBS- or wt IL-10-injected mice exhibited severe inflammation in the paws (FIG. 17A). From histological analysis, intravenous administration of SA-IL-10 significantly suppressed the inflammatory responses in the paws compared with PBS-treated mice and reduced joint pathology (FIG. 17B). The effect of the administration route on therapeutic efficacy was also investigated, comparing intravenous, local (footpad), and subcutaneous (at a distant site, mid-back) administration (FIG. 17C). Strikingly, SA-IL-10 showed quite high suppression effects on CAIA by all of the administration routes tested (FIG. 17C).

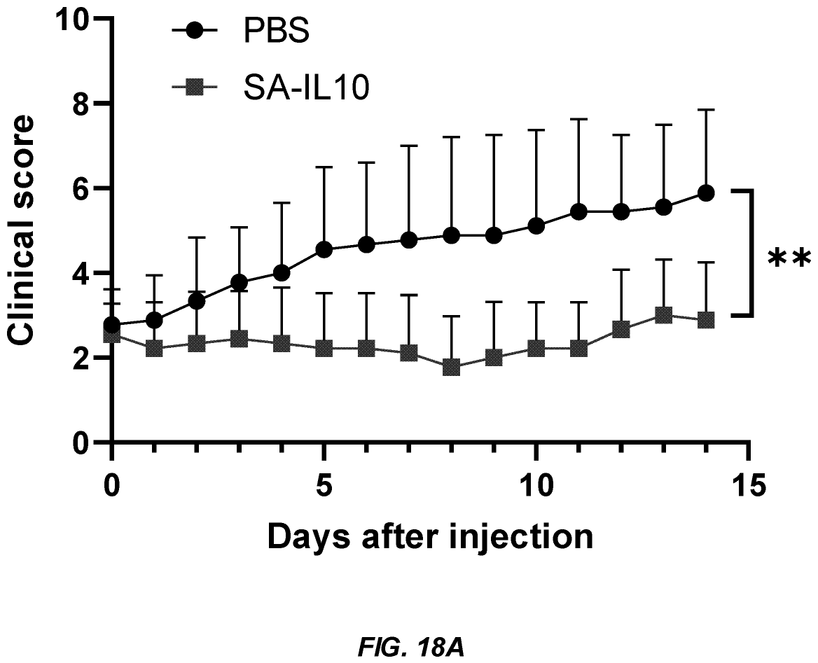

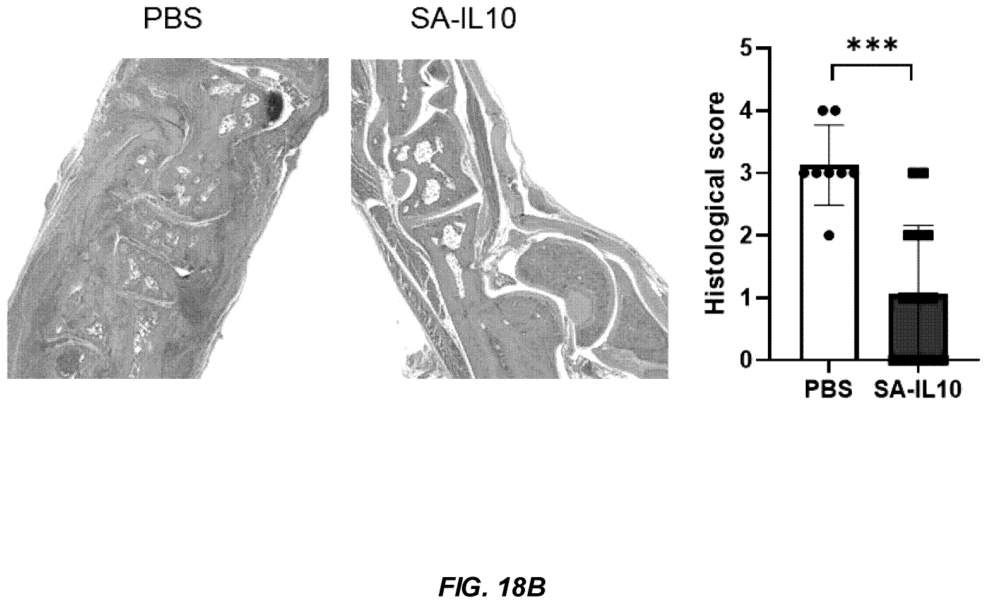

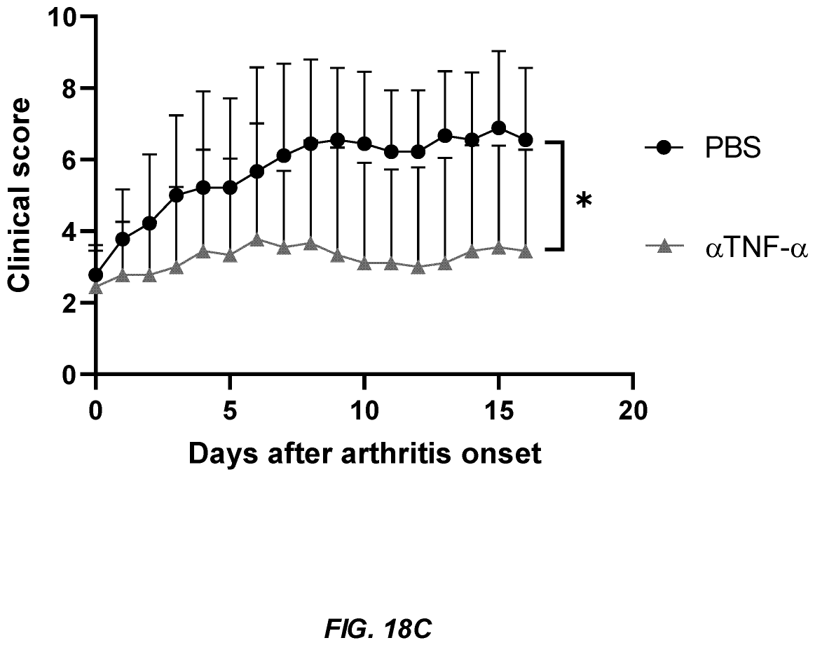

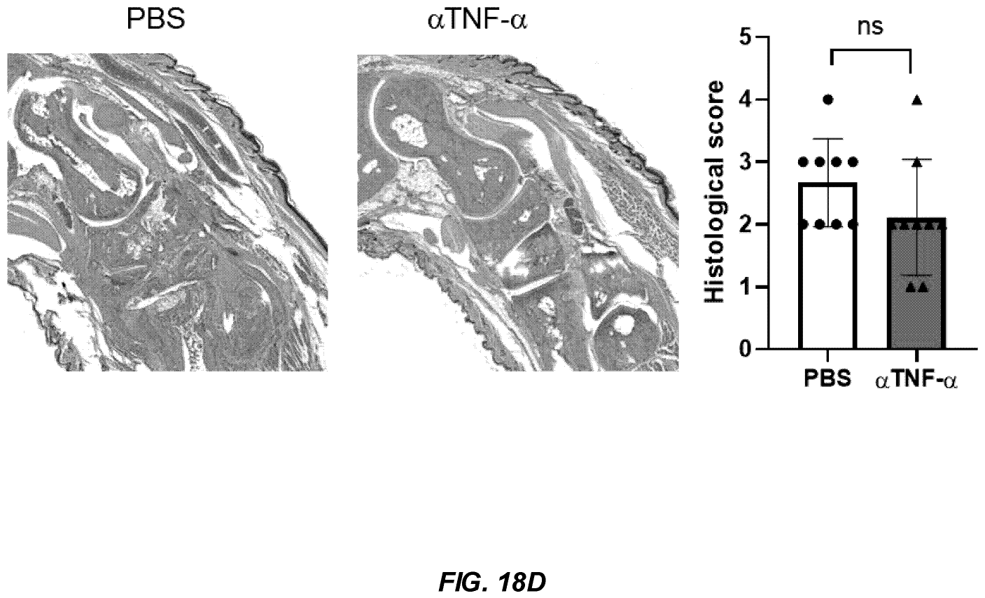

As a second arthritis model, the active collagen-induced arthritis (CIA) model was used for evaluation of SA-IL-10 on RA treatment. A single injection of SA-IL-10 to CIA mice induced significant suppression of establishment of arthritis compared with PBS (FIG. 18A). Most mice treated with PBS showed severe inflammation at the paws as indicated by histology and the histological score (FIG. 18B). In contrast, SA-IL-10-treated mice exhibited almost identical status in the paw as naïve mice, and most mice showed a histological score of 1 or less (FIG. 18B). The treatment with anti-TNF-α antibody (αTNF-α), a mouse model of a clinically used antibody drug for treatment of RA, also suppressed the increase of clinical scores compared with PBS-treated CIA mice (FIG. 18C), however twice αTNF-α injection did not restore the histology of the joints and histological scores (FIG. 18D). Taken together, these results indicated the highly suppressive effect of inflammation by local or intravenous administration and by even subcutaneous administration of SA-IL-10 and that its therapeutic effect was comparable with or even superior to αTNF-α treatment.

5. Albumin-Fused IL-10 Suppresses Inflammatory Responses in the Paws

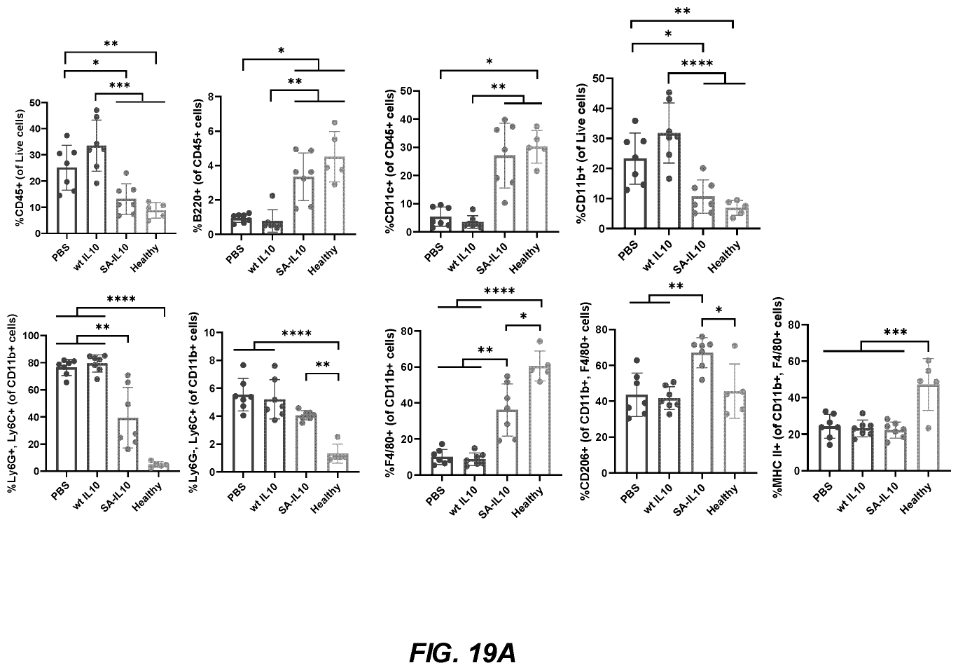

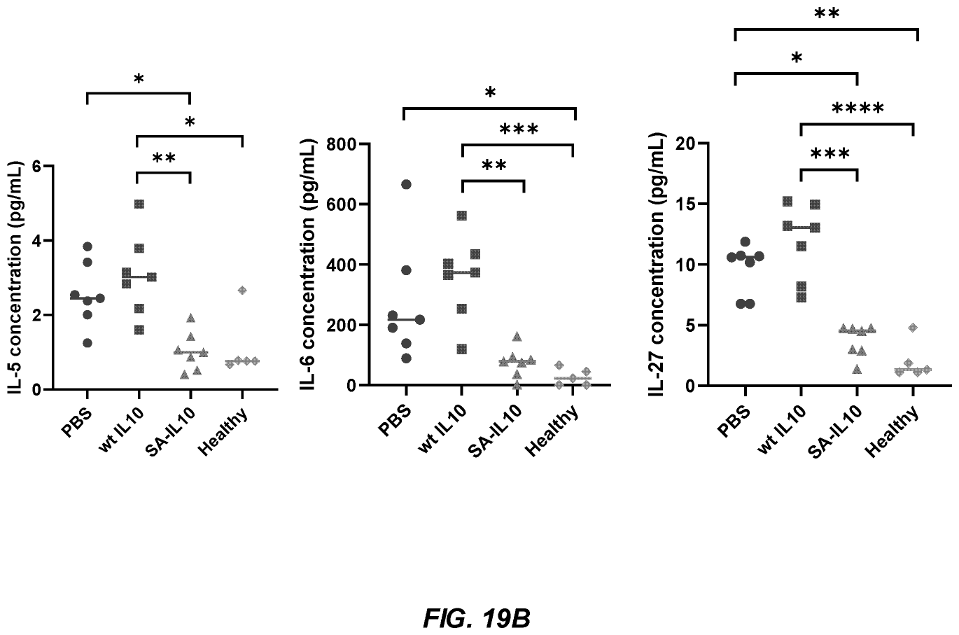

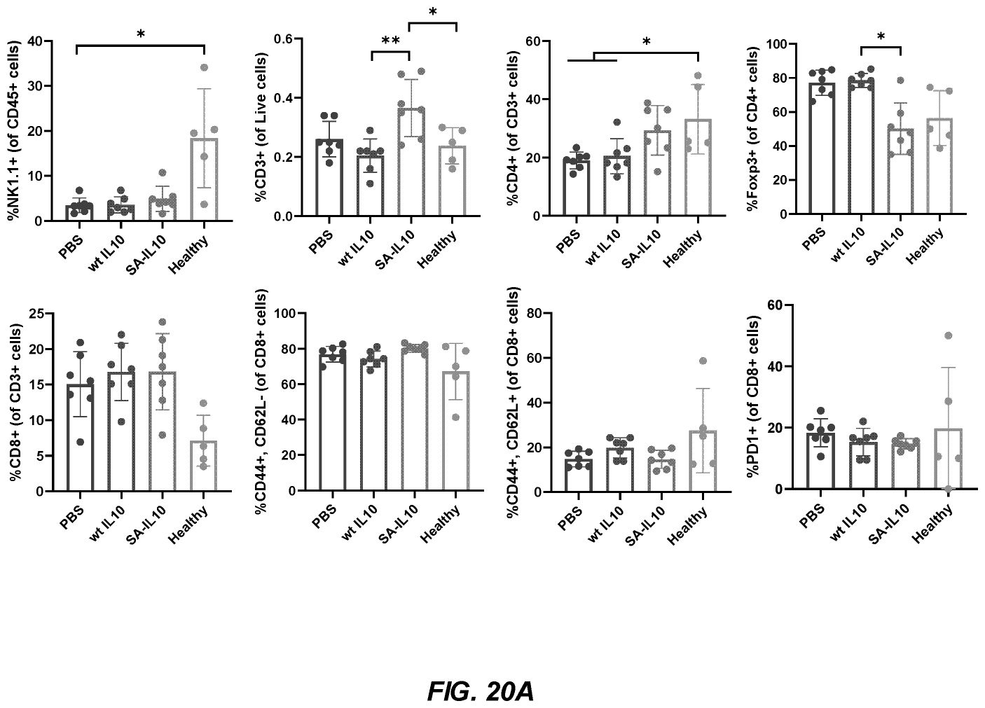

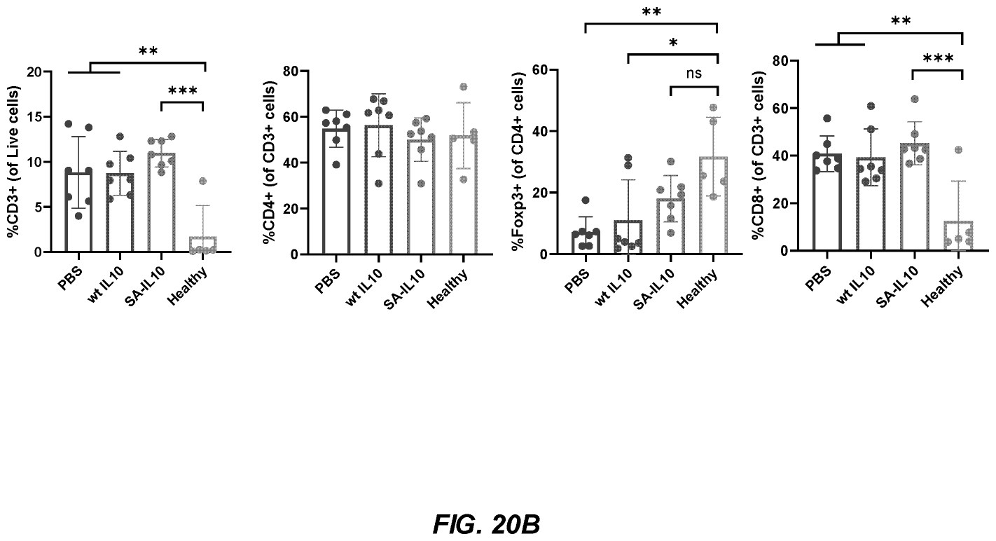

Next, immune cell populations in the hind paws were analyzed using flow cytometry (FIG. 19A). After intravenous injection of SA-IL-10, frequencies of CD45⁺ immune cells were significantly decreased compared with PBS- or wt IL-10-treated groups. Within CD45⁺ cells, the frequencies of B cells and dendritic cells became comparable to levels in healthy mice, and CD11b⁺ cells were remarkably decreased to the level of healthy mice as well. Among CD11b⁺ cells, the G-MDSC population was reduced, and the macrophage frequency was recovered to the level of healthy mice. In addition, the frequency of CD206⁺ M2 macrophages was significantly increased by injection of SA-IL-10 compared with PBS or wt IL-10 treatment, even exceeding that of healthy mice. The analysis of T cell populations in the paws revealed that SA-IL-10 suppressed the change in CD4⁺ cells and Foxp3⁺ Treg in CAIA mice (FIG. 20A). Furthermore, SA-IL-10 suppressed a decrease of the Treg frequency in the blood (FIG. 20B). Reflecting these changes in immune cell populations, various inflammatory cytokines in the paws were significantly decreased by intravenous injection of SA-IL-10, which levels were comparable with those in healthy mice (FIG. 19B).

6. Albumin-Fused IL-10 Shows No Toxicity after Injection

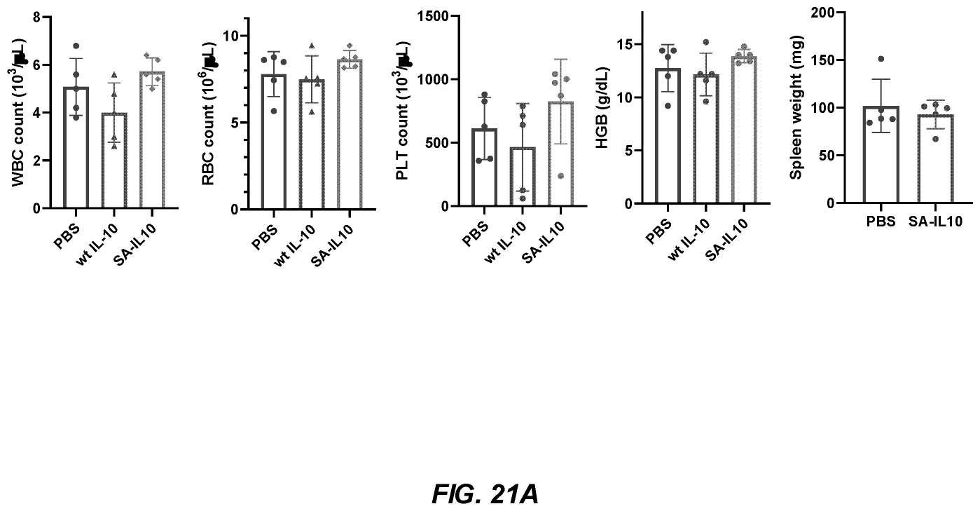

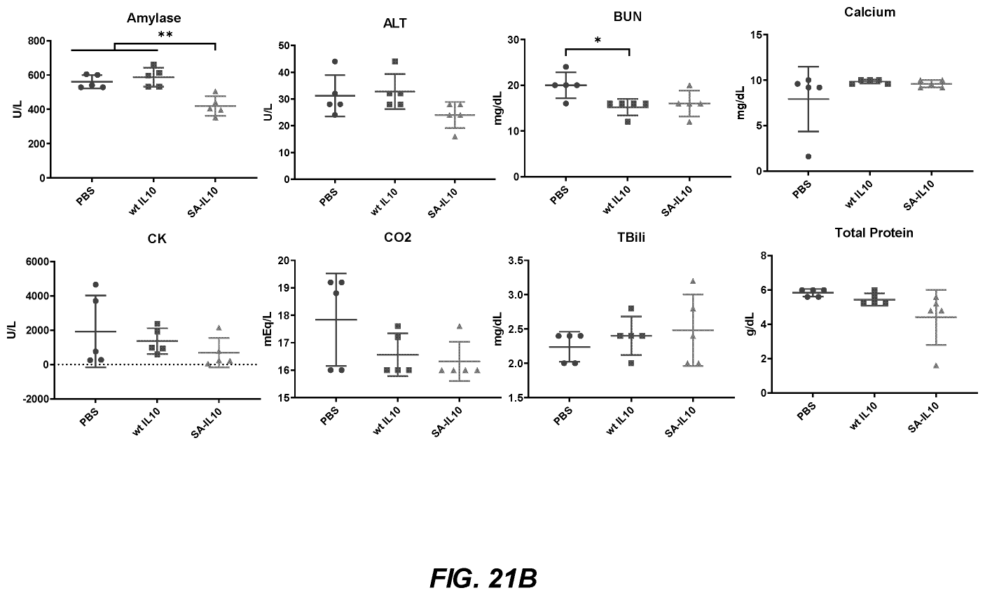

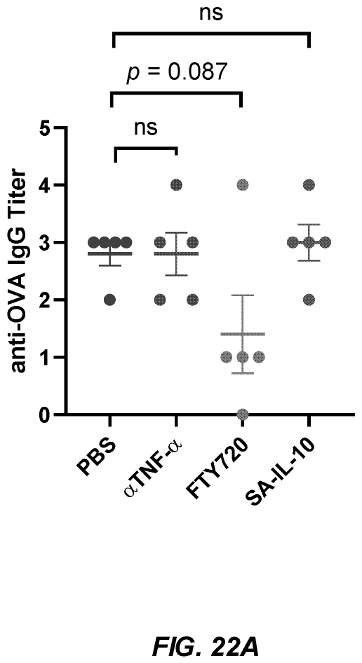

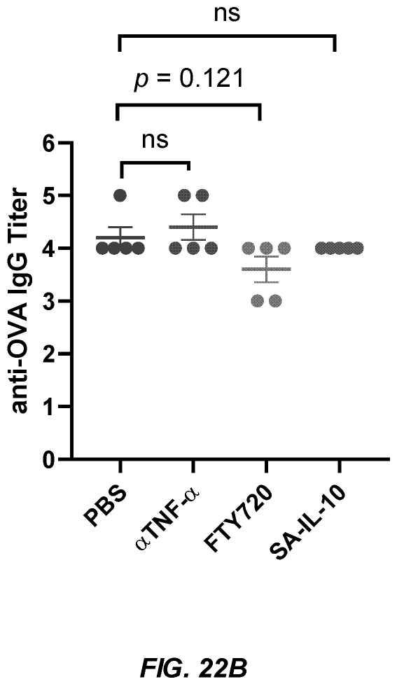

Finally, safety assessments were performed to investigate whether engineered IL-10 demonstrates any adverse effects. Representative blood parameters measured by a hematology analyzer and spleen weights did not show any significant changes among the treatment groups (FIG. 21A). Various biochemical markers in serum were also investigated using a biochemistry analyzer (FIG. 21B). For the engineered IL-10-treated groups, most markers, except for amylase (which was not increased, rather slightly decreased), showed similar levels compared with PBS-treated group, indicating that engineered IL-10 possesses high safety after systemic administration. Furthermore, injection of SA-IL-10 did not affect anti-OVA IgG titer, comparable to results observed with αTNF-α, whereas FTY720, a clinically approved drug (fingolimod) for treating multiple sclerosis, showed some immunosuppressive effect (but not significant) under experimental condition (FIGS. 22A and 22B). These results indicate that engineered IL-10 possesses high safety after systemic administration.

B. Materials and Methods

1. Production and Purification of Recombinant Proteins

The sequences encoding for the mouse serum albumin without pro-peptide (25 to 608 amino acids of whole serum albumin), mouse IL-10, and a (GGGS)₂ linker were synthesized (SEQ ID NO:X15) and subcloned into the mammalian expression vector pcDNA3.1(+) by Genscript. A sequence encoding for 6 His was added at the C-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×10⁶ cells/mL. 2 μg/mL plasmid DNA, 2 μg/mL linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% C02. Seven days after transfection, the cell culture medium was collected by centrifugation and filtered through a 0.22 μm filter. Culture media was loaded into a HisTrap HP 5 mL column (GE Healthcare), using an ÄKTA pure 25 (GE Healthcare). After washing the column with wash buffer (20 mM NaH₂PO₄, 0.5 M NaCl, pH 8.0), protein was eluted with a gradient of 500 mM imidazole (in 20 mM NaH₂PO₄, 0.5 M NaCl, pH 8.0). The protein was further purified with size exclusion chromatography using a HiLoad Superdex 200PG column (GE Healthcare) using PBS as an eluent. All purification steps were carried out at 4° C. The expression of the proteins was verified as >90% pure by SDS-PAGE. Purified proteins were tested for endotoxin via HEK-Blue TLR4 reporter cell line and endotoxin levels were confirmed to be less than 0.01 EU/mL. Protein concentration was determined through absorbance at 280 nm using NanoDrop (Thermo Scientific).

2. Detection of SA-IL-10 Binding to FcRn

SPR measurements were carried out with Biacore X100 instrument. Recombinant mouse FcRn (Acro Biosystems) was immobilized via amine coupling on a C1 chip (GE Healthcare) for ˜200 RU according to the manufacturer's instructions. SA-IL-10 was flowed at decreasing concentrations in the running buffer (0.01 M monobasic anhydrous sodium phosphate, pH 5.8, 0.15 M NaCl) at 30 μL/min at room temperature. The sensor chip was regenerated with PBS, pH 7.4 for every cycle. Specific binding of SA-fused proteins to FcRn was calculated by comparison to a non-functionalized channel used as a reference. The K_{d} value of the SA-IL-10 was determined by fitting 1:1 Langmuir binding model to the data using BIAevaluation software (GE Healthcare).

3. Mice

BALB/c female mice at 7 wk of age and DBA/1J male mice at 8 wk of age were obtained from the Jackson Laboratory. Experiments were performed with approval from the Institutional Animal Care and Use Committee of the University of Chicago.

4. Binding of the Proteins to Splenocytes or LN-Derived Cells

Single-cell suspensions were obtained by gently disrupting the spleen and popliteal LN through a 70-μm cell strainer. Red blood cells were lysed with ACK lysing buffer (Quality Biological) for splenocytes. Cells were counted and re-suspended in RPMI-1640 supplemented with 10% FBS and 1% penicillin/streptomycin (all from Life Technologies). 1×10⁵ cells/well were seeded in a 96 well microplate and were incubated with 2 μg/100 μL of SA, or SA-IL-10 for 30 min on ice. After 4-times washing by PBS, cells were further incubated with anti-mouse albumin antibodies (abcam) for 20 min on ice. After 3-times washing by PBS, cells were incubated with 1 μg/mL AlexaFluor 647-labeled anti-Rabbit IgG (Jackson ImmunoResearch), anti-B220 (RA3-6B2, BioLegend), anti-CD3e (145-2C11, BD Biosciences), anti-CD4 (RM4-5, BD Biosciences), anti-CD8 (53-6.7, BD Biosciences), anti-CD11c (HL3, BD Biosciences), anti-CD45 (30-F11, BD Biosciences) and anti-F4/80 (T45-2342, BD Biosciences) antibodies for 20 min on ice. Cells were analyzed by flow cytometry as described below.

5. Plasma Pharmacokinetics of the Proteins

IL-10 or SA-IL-10 (equivalent to 35 μg of IL-10) was injected intravenously into female BALB/c mice. Blood samples were collected in protein-low binding tubes at 1, 5, 10, and 30 min, and 1, 4, 8 and 24 hr after injection, followed by overnight incubation at 4° C. IL-10 concentrations in serum were measured by IL-10 Mouse Uncoated ELISA kit (Invitrogen) according to the manufacturer's protocol. Exponential two-phase decay (Y=Ae^{-α}^{ₜ}+Be^{-β}^{ₜ}) fitting was used to calculate the half-life. Fast clearance half-life, t_{1/2,α}; slow clearance half-life, t_{1/2,β}. Data were analyzed using Prism software (v8, GraphPad).

6. CAIA Model

Arthritis was induced in female BALB/c mice by intraperitoneal injection of anti-collagen antibody cocktail (1.0 mg/mouse, Chondrex) on day 0, followed by intraperitoneal injection of LPS (25 μg/mouse, Chondrex) on day 3. On the day 3, mice were intravenously, subcutaneously (mid-back), or via footpad injected with PBS, wt IL-10, SA-IL-10 (each equivalent to 43.5 μg of IL-10), or 200 μg of Rat anti-mouse TNF-α antibody (clone XT3.11, Bio X Cell) before LPS injection. Joint swelling was scored every day according to the manufacture's protocol (Chondrex). On the last day of scoring, the hind paws were fixed in 10% neutral formalin (Sigma-Aldrich), decalcified in Decalcifer II (Leica), and then provided for histological analysis. Paraffin-embedded paws were sliced at 5 am thickness and stained with H&E. The images were scanned with a Pannoramic digital slide scanner and analyzed using a Pannoramic Viewer software. The severity of synovial hyperplasia and bone resorption for the arthritis model was scored by three-grade evaluation (0-2) according to the previously reported criteria with slight modifications as follows: 0, normal to minimal infiltration of pannus in cartilage and subchondral bone of marginal zone; 1, mild to moderate infiltration of marginal zone with minor cortical and medullary bone destruction; 2, severe infiltration associated with total or near total destruction of joint architecture. The scores in both hind paws were summed for each mouse (score per mouse total, 0-4). The histopathological analyses were performed in a blinded fashion.

7. CIA Model

Male DBA/1J mice (8 wk old) were immunized by subcutaneous injection at the base of the tail with bovine collagen/complete Freund's adjuvant (CFA) emulsion (Hooke Kit, Hooke Laboratories). Three weeks later, a booster injection of bovine collagen/incomplete Freund's adjuvant (IFA) emulsion (Hooke Kit, Hooke Laboratories) was performed. After the booster injection, mice were inspected every day, and joint swelling was scored according to the manufacture's protocol (Hooke Laboratories). When showing total score of 2-4 (defined as Day 0), mice were intravenously injected with PBS, SA-IL-10 (each equivalent to 43.5 μg of IL-10), or 200 μg of Rat anti-mouse TNF-α antibody (clone XT3.11, Bio X Cell). On the last day of scoring, hind paws were collected and histological analyses were employed as described above.

8. In Vivo Bio-Distribution Study

To make fluorescently labeled protein, wt IL-10, and SA-IL-10 were incubated with 8-fold molar excess of using DyLight 800 NHS ester (Thermo Fisher) for 1 hr at room temperature, and unreacted dye was removed by a Zebaspin spin column (Thermo Fisher) according to the manufacturer's instruction. BALB/c mice were intraperitoneal injected by anti-collagen antibody cocktail (1.0 mg/mouse) on day 0, subsequently 10 μg of LPS was injected to right hind paw on day 3. The following day, 20 μg of DyLight 800-labeled proteins were intravenously injected. After 4 hr, organs harvested from the disease model were imaged with the Xenogen IVIS Imaging System 100 (Xenogen) under the following conditions: f/stop: 2; optical filter excitation 745 nm; excitation 800 nm; exposure time: 5 sec; small binning. Each organ was weighed to normalize the fluorescence signal from each organ.

9. LN Microscopy

BALB/c mice were intravenously injected with DyLight594-labeled wt IL-10 (43.5 g) or SA-IL-10 labeled with equimolar amounts of dye. 24 hr after injection, popliteal LNs were harvested and frozen in dry ice with optimal cutting temperature (OCT) compound. Tissue slices (10 m) were obtained by cryo-sectioning. The tissues were fixed with 2% paraformaldehyde in PBS for 15 min at room temperature. After washing with PBS-T, the tissues were blocked with 2% BSA in PBS-T for 1 hr at room temperature. The tissues were stained with anti-mouse CD3 antibody (1:100, 145-2C11, BioLegend) or anti-mouse peripheral node addressing (PNAd) antibody (1:200, MECA79, BioLegend) and Alexa Fluor 488 donkey anti-rat (1:400, Jackson ImmunoResearch). The tissues were washed three times and then covered with ProLong gold antifade mountant with 4′, 6-diamidino-2-phenylindole (DAPI; Thermo Fisher Scientific). An IX83 microscope (Olympus) was used for imaging with 10× magnification for CD3 staining, and a Leica SP8 3D Laser Scanning Confocal microscope with 20× magnification for PNAd staining. Images were processed using ImageJ software (NIH).

10. LN Pharmacokinetics

wt IL-10, or SA-IL-10 (each equivalent to 35 μg of IL-10) was injected intravenously into CAIA mice. Popliteal, mesenteric, cervical LNs were collected at 30 min, and 1, 4, 8 and 24 hr after injection, and were subsequently homogenized using Lysing Matrix D and FastPrep-24 5G (MP Biomedical) for 40 s at 5,000 beats/min in T-PER tissue protein extraction reagent (Thermo Scientific) with cOmplete™ proteinase inhibitor cocktail (Roche). After homogenization, samples were incubated overnight at 4° C. Samples were centrifuged (5,000 g, 5 min), and the total protein concentration and IL-10 concentration were analyzed using a BCA assay kit (Thermo Fisher) and IL-10 Mouse Uncoated ELISA kit (Invitrogen), respectively. Simultaneously, cytokine levels in the LN extract were measured using Mouse Uncoated ELISA kit (Invitrogen) or Ready-SET-Go! ELISA kits (eBioscience) according to the manufacturer's protocol. For detection of GM-CSF, wt IL-10 or SA-IL-10 (each equivalent to 35 μg of IL-10) was injected intravenously twice with a 3 day interval into CAIA mice. The day following the last injection, popliteal LNs were collected for detection of GM-CSF.

11. Flow Cytometry

CAIA mice were intravenously injected with PBS, wt IL-10, or SA-IL-10 (each equivalent to 43.5 μg of IL-10). Eight days after, blood and hind paws were harvested. Red blood cells in blood were lysed with ACK lysing buffer (Quality Biological), followed by antibody staining for flow cytometry. Paws were digested in Dulbecco's Modified Eagle Medium (DMEM) supplemented with 2% FBS, 2 mg/mL collagenase D and 40 μg/mL DNase I (Roche) for 60 min at 37° C. Single-cell suspensions were obtained by gently disrupting through a 70-μm cell strainer. Antibodies against the following molecules were used: anti-mouse CD3 (145-2C11, BD Biosciences), CD4 (RM4-5, BD Biosciences), anti-mouse CD8a (53-6.7, BD Biosciences), anti-mouse CD25 (PC61, BD Biosciences), anti-mouse CD45 (30-F11, BD Biosciences), CD44 (IM7, BD Biosciences), CD62L (MEL-14, BD Biosciences), PD-1 (29F.1A12, BD Biosciences), NK1.1 (PK136, BD Biosciences), Foxp3 (MF23, BD Biosciences), F4/80 (T45-2342, BD Biosciences), MHC II (M5/114.15.2, BioLegend), CD206 (C068C2, BioLegend), Ly6G (1A8, BioLegend), Ly6C (HK1.4, BioLegend), CD11b (M1/70, BioLegend), CD11c (HL3, BD Biosciences), B220 (RA3-6B2, BioLegend). Fixable live/dead cell discrimination was performed using Fixable Viability Dye eFluor 455 (eBioscience) according to the manufacturer's instructions. Staining was carried out on ice for 20 min if not indicated otherwise, and intracellular staining was performed using the Foxp3 staining kit according to manufacturer's instructions (BioLegend). Following a washing step, cells were stained with specific antibodies for 20 min on ice prior to fixation. All flow cytometric analyses were done using a Fortessa (BD Biosciences) flow cytometer and analyzed using FlowJo software (Tree Star).

12. Safety Assessments

BALB/c mice were intravenously injected with PBS, wt IL-10, or SA-IL-10 (each equivalent to 43.5 μg of IL-10). Two days after injection, blood samples collected from mice were analyzed using a COULTER Ac⋅T 5diff CP hematology analyzer (Beckman Coulter) according to the manufacturer's instructions. Spleen weight was also measured. Serum samples collected from protein-injected mice were analyzed using Biochemistry Analyzer (Alfa Wassermann Diagnostic Technologies) according to the manufacturer's instructions. For evaluation of general immunosuppression, PBS and 100 μg anti-TNF-α were injected intraperitoneally every two days beginning on day 0 for 14 days. FTY720 (1 mg/kg body weight) was administered orally every day. SA-IL-10 (equivalent to 43.5 μg of IL-10) was injected subcutaneously on days 0 and 8. C57BL/6 mice were challenged subcutaneously in the front hocks on day 5 with 10 μg endotoxin-free ovalbumin, 50 μg alum, and 5 μg monophosphoryl lipid A (MPLA). Mice were bled on days 13 and 19, and plasma was analyzed for anti-ovalbumin total IgG titers.

13. Statistical Analysis

Statistically significant differences between experimental groups were determined using Prism software (v8, GraphPad). Where one-way ANOVA followed by Tukey's HSD post hoc test was used, variance between groups was found to be similar by Brown-Forsythe test. For non-parametric data, Kruskal-Wallis test followed by Dunn's multiple comparison test was used. For single comparisons, a two-tailed Student's t-test was used. The symbols *, **, *** and **** indicate P values less than 0.05, 0.01, 0.001 and 0.0001 respectively; ns, not significant.

### Example 3—Albumin-Fused IL-35 Suppressed Arthritis Development

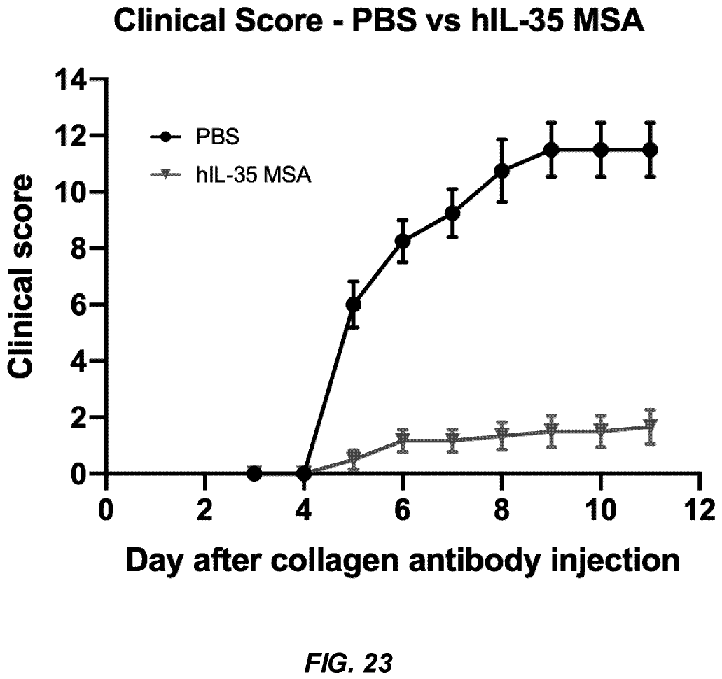

Arthritis (CAIA) was induced by passive immunization with anti-collagen antibodies, followed by intraperitoneal injection of LPS. On the day of LPS injection, PBS, or human IL-35-mouse SA fusion protein (equivalent to 54 μg of wt IL-35) was injected intravenously into the arthritic mice. The arthritis scores for each group are shown in FIG. 23. SA-IL-35 significantly reduced disease progression. Arthritis scores represent the mean+SEM from 7 mice. Statistical analyses were done using a two-tailed Student's t-test, and p value was <0.01 between PBS and IL-35-MSA.

### Example 4—SA-IL-4 Promotes Wound Healing Through Angiogenesis

Full-thickness back-skin wounds were made in db/db type 2 diabetic mice. After 7 days, wound closure and blood vessel number in granulation tissue area were evaluated by histomorphometry. Wounded db/db diabetic mice were injected every other day for 6 days from day 0. Treatment groups were: phosphate-buffered saline (PBS) subcutaneously (s.c.), 10 μg wild-type (WT) IL-4 subcutaneous., or SA-IL-4 (equimolar: 10 μg, IL-4 base). Graphical data were mean±standard error of the mean (SEM), Statistical comparisons were carried out using one-way ANOVA *P<, 0.05; **P<, 0.01.

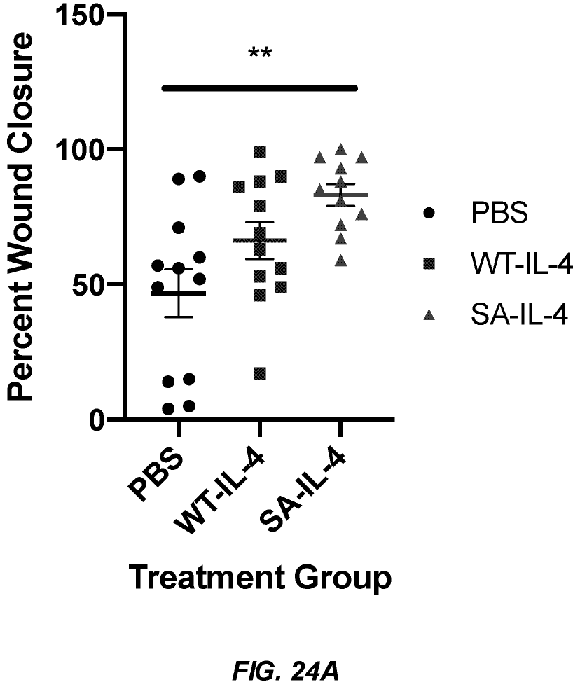

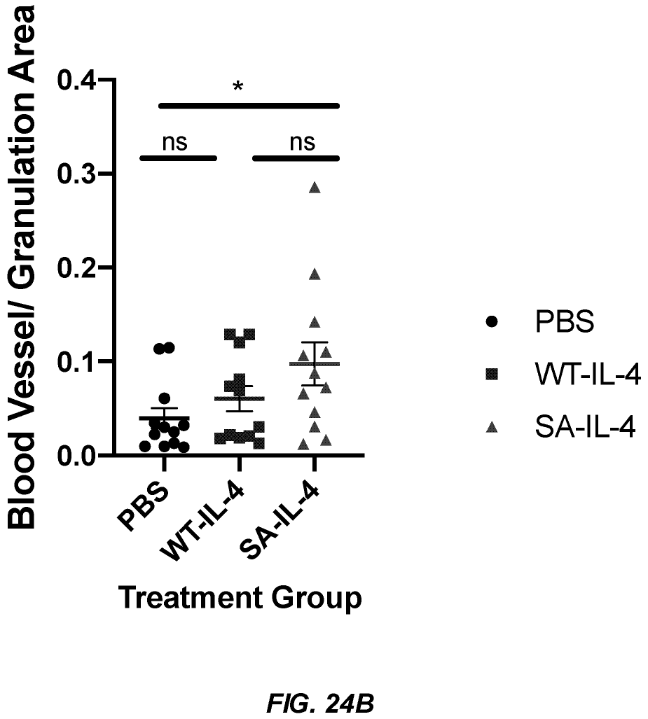

FIG. 24A shows wound closure results for all treatment groups. SA-IL-4 treatment significantly improved wound closure. FIG. 24B shows results for blood vessel number in granulation tissue. SA-IL-4 treatment significantly improved blood vessel number, indicating an increase in angiogenesis.

### Example 5—SA-IL-27 Reduces Plasma IFNγ Concentration after Cancer Immunotherapy in Tumour-Bearing Mice

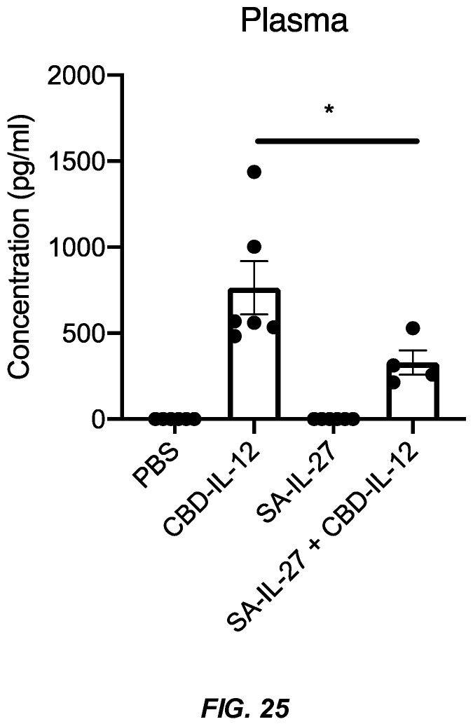

Plasma IFNγ concentration represents serum cytokine release syndrome after cancer immunotherapy. Seven days before cytokine treatment, 5×10⁵ B16F10 melanoma cells were inoculated and mice were treated i.v. with CBD-IL-12 (25 μg, IL-12 based) and SA-IL-27 (10 μg, IL-27 based) once on day 0. Plasma IFNγ levels on day 2 are shown in FIG. 25. SA-IL-27 significantly reduced IFNγ levels versus mice treated with CBD-IL-12 alone. *=P<0.05 CBD-IL-12: collagen binding domain fused IL-12 recombinant protein.

### Example 6—Use of Albumin-Fused Cytokines for Wound Healing

As of 2020, 34.2 million people have been diagnosed with diabetes mellitus, ‘diabetes’, and is one of the leading causes of death in the United States. Beyond mortality, there are many complications that are often associated with diabetes, one of which is an impaired ability to heal. This most commonly manifests in wounds on the lower extremities (i.e., ulceration of the feet). These ulcerations can lead to amputation, a procedure for which diabetic patients make up 85% of the patient population. Wound healing, especially within the context of diabetes, is a problem that has substantial work surrounding it but currently lacks a satisfying solution. Current clinical therapies are severely limited to management strategies such wound offloading and surgical debridement as opposed to long-term solutions. The few treatments available have shown little improvement on patient outcomes. For example, current work focuses on the use of growth factors, such as vascular endothelial growth factor A (VEGF-A), a factor associated with neoangiogenesis. VEGF-A treatment has shown some improvement in re-epithelization of chronic wounds though VEGF-A has been associated with unfavorable side effects such as sustained vascular leakage leading to hypotension. Genentech developed a topical recombinant VEGF-A treatment, telbermin, that has not been clinically approved.

The inventors' approach to improved healing of chronic wounds focuses on the immunomodulation of the diabetic wound environment. Many immune cell populations are involved in orchestrating the closure of wounds, and in diabetic wounds many of these immune cells are dysregulated. Monocytes and macrophages are crucial players in normal skin wound healing but are impaired in a couple of ways within a diabetic wound environment. Diabetic wounds demonstrate an increased number of both monocytes and macrophages as well as an inability to transition from pro-inflammatory macrophages to anti-inflammatory macrophages. This failed transition from pro-inflammatory macrophages to anti-inflammatory macrophages is the target of the engineered cytokine treatment. Both interleukin-4 (IL-4) and interleukin-10 (IL-10) are widely regarded as anti-inflammatory cytokines. Both IL-4 and IL-10 are also key players in the pathway that induces the polarization of macrophages from a pro-inflammatory phenotype to an anti-inflammatory phenotype. The inventors have made engineering changes to both IL-4 and IL-10 to increase their circulation half-life and local wound retention, respectively. These novel engineered cytokine constructs have demonstrated the ability to improve wound closure through re-epithelialization in a mouse model of diabetes.

A. Methods

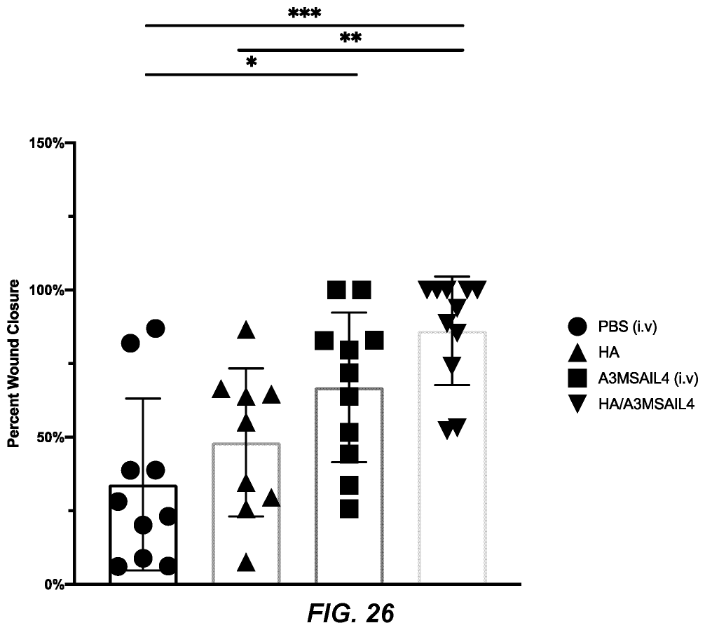

Male db/db mice ages 8-10 weeks from Jackson laboratories were used. The back of each mouse was widely shaved and cleaned using 70% ethanol and iodine. Using a 6-millimeter diameter biopsy punch 4 symmetric, full thickness wounds were made on the shaved area. Immediately following, the wounds were dressed with non-adhesive Adaptic™ dressing and adhered with a Tegaderm™ seal and tissue adhesive. On day four cytokine therapies were administered, 40 ug IL-10 molar equivalent of MSA-IL-10 was given subcutaneously, and 200 ug IL-4 molar equivalent of A3-MSA-IL-4 was applied topically with a hyaluronic acid hydrogel carrier. The wounds of mice in FIG. 26 were splinted open to prevent contraction. On day 11 mice were euthanized and wound excised for histological analysis.

B. Results

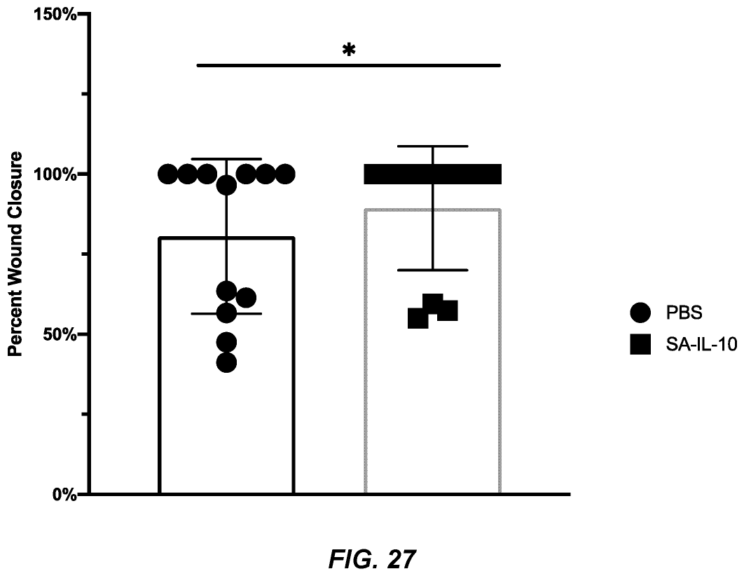

After excision of wounds and histological analysis the inventors were able to determine that the engineered cytokine therapies resulted in meaningful increases in wound closure as defined by re-epithelization when compared to both the PBS control and in the case of A3-MSA-IL-4 the hyaluronic acid only control as well. These results are shown in FIGS. 26-27.

### Example 7: Use of Albumin Fused Cytokines for Scleroderma Therapy

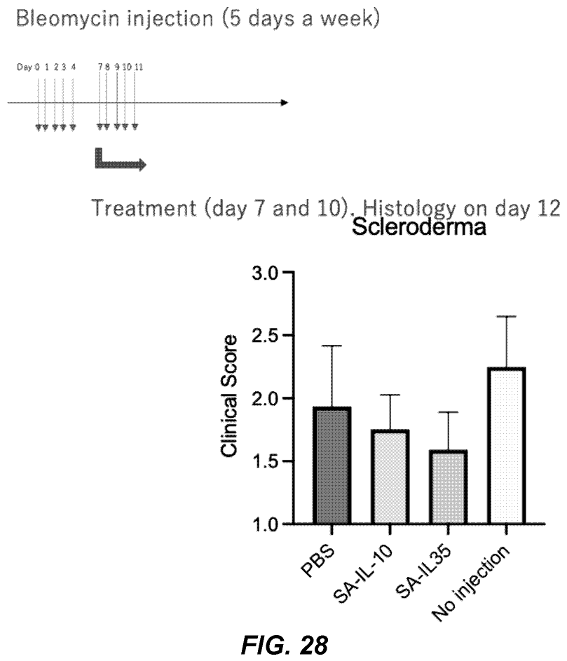

Scleroderma is a chronic autoimmune disease in which skin tissue is replaced with thick tissue with excess collagen. Cytokine therapies for scleroderma has not been tested previously. The inventors sought to test out SA-mouse IL10 and SA-human IL-35 fusion proteins to assess if they show any effects. First, they dissolved bleomycin in PBS at a concentration of 1 mg/ml. Then, anesthetized mice by isoflurane. Put hair remover lotion on their back, and waited for 30-60 sec and remove the lotion and their hair by tissue papers. They next injected bleomycin (100 μg=100 μl) subcutaneously into a single location on the back of the mice 5 days a week for 2 weeks with a 27-gauge needle. The inventors treated mice with SA-IL-10 (40 μg/injection) or 20 μg/injection on days 7 and 10 s.c. As a control, they injected PBS 100 μl s.c. with a 27-gauge needle. Lastly, they drew a circle to mark the injection sites with a marking pen. The graph of FIG. 28 depicts the clinical score of scleroderma, measured by histology blindly. Mean+SD.

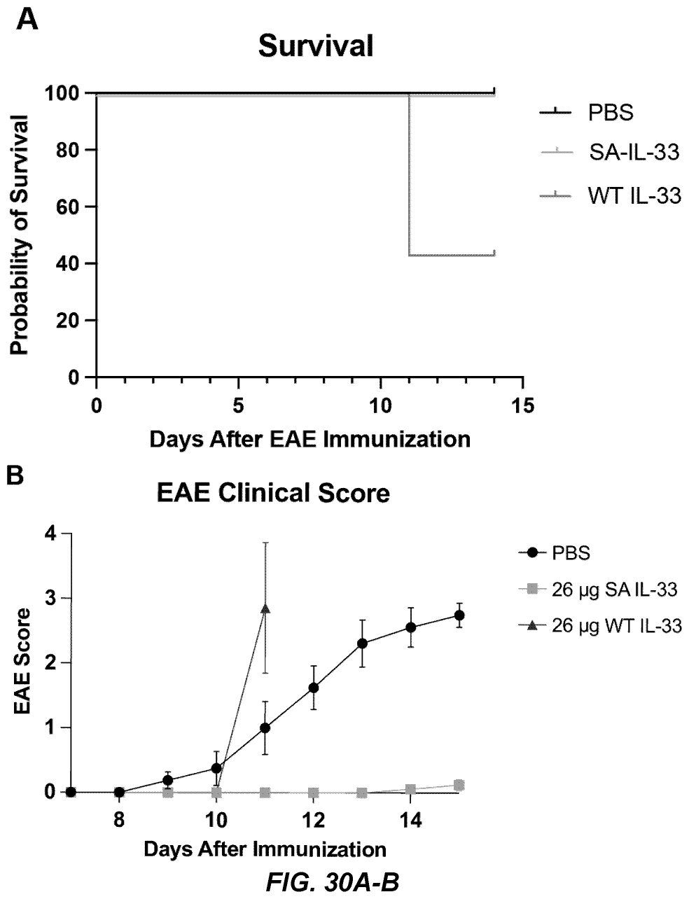

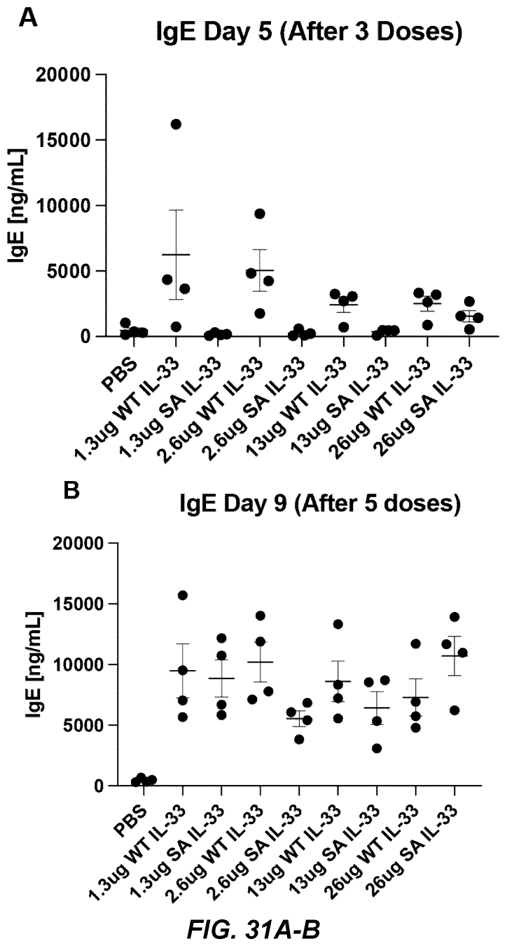

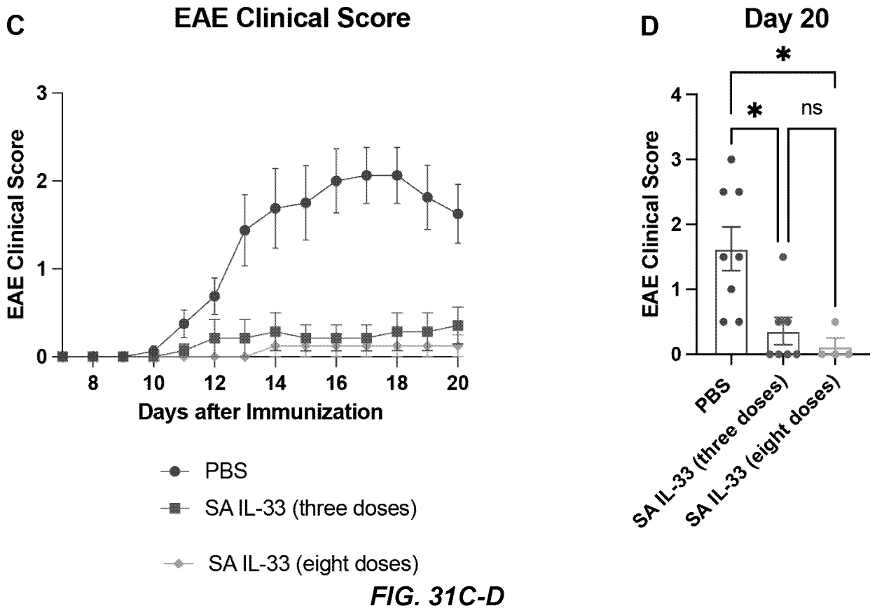

These studies demonstrate that SA IL-33 substantially reduces toxicity compared to wild type IL-33. Mice were induced with EAE at day 0 and received subcutaneous wild type or equimolar SA IL-33 every other day beginning at day 8. At day 11, after two treatments, mice receiving wild type IL-33 experienced severe toxicity and death (FIG. 30A). In mice receiving SA-IL-33, no death was observed, and mice continued to receive SA-IL-33 and were protected from the development of EAE (FIG. 30B). In healthy mice, dosing every other day for a total of 3 doses had little effect on production of serum IgE, compared to wild type IL-33 (FIG. 31A). After 5 doses, mice treated with SA-IL-33 had equivalent levels of serum IgE as those treated with wild type IL-33 (FIG. 31B). To determine if three doses was sufficient to protect from EAE, mice were induced with EAE and treated with SA-IL-33 every other day beginning on day 8 after induction. Three doses were sufficient to protect from EAE compared to 8 doses, suggesting that a three dose regimen would be sufficient to protect from disease (FIGS. 31C-D).

### Example 8: Management of Toxicity of IL-4 and IL-33 by SA-Fusion and Dose Selection

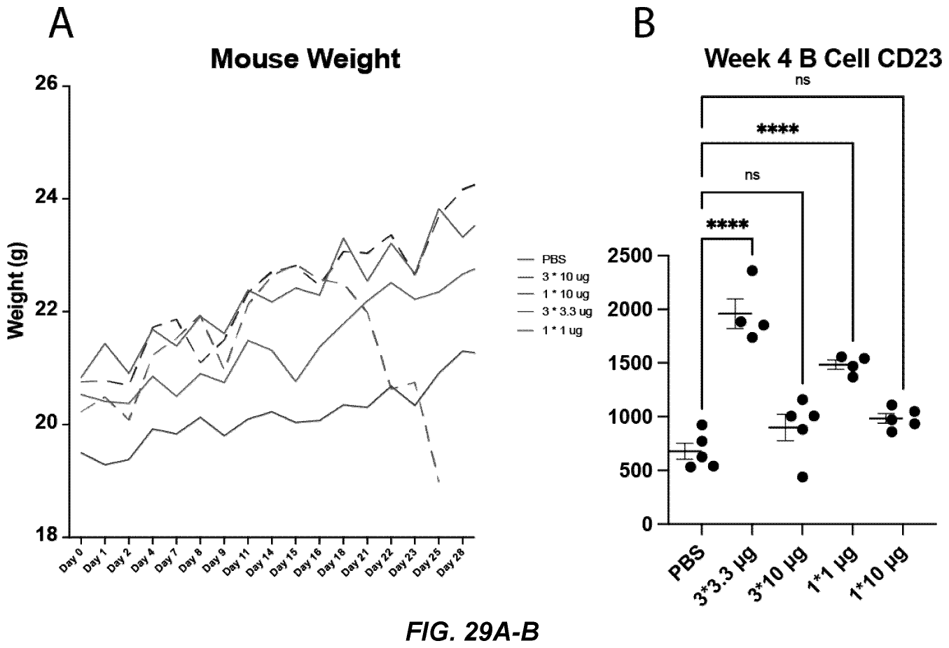

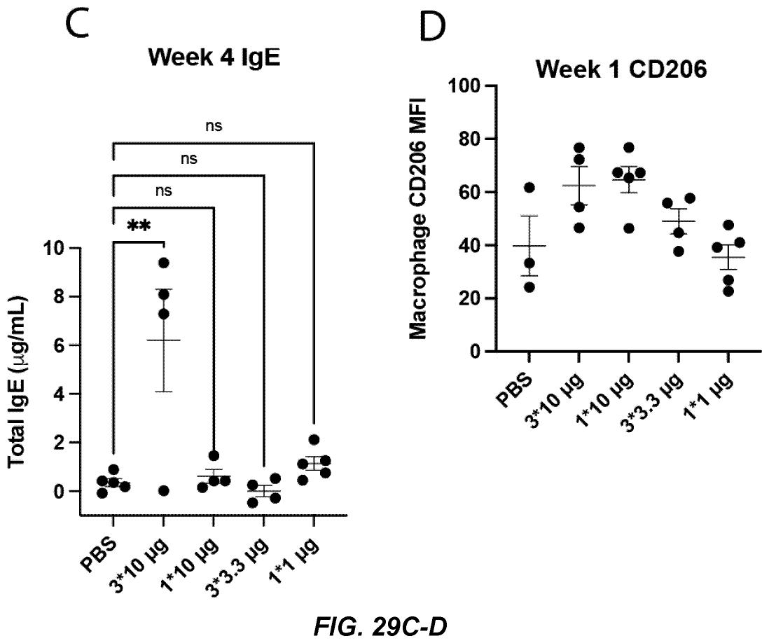

IL-4 is a pleiotropic cytokine that has been used as a potential therapeutic in cancer patients with malignant melanoma and metastatic renal carcinoma. In these studies, there were a notable number of patients with grade 3 or 4 toxicities requiring cessation of treatment. The dosing strategy for these studies included daily or thrice weekly doses of low dose subcutaneous wild type IL-4. Similarly, in mice constitutively overexpressing IL-4, there are toxicities associated with overactivation of B cells as well as a hyper-IgE response. The inventors demonstrate that more frequent dosing, i.e. thrice weekly, leads to a higher level of side effects including weight loss, B cell activation, and serum IgE levels, compared to less frequent (one weekly) dosing (FIG. 29A-C). Importantly, the inventors observe that a single low dose of SA-IL-4 is sufficient to drive upregulation of the CD206 mannose receptor, an indication of efficacy at this dosing level. (FIG. 29D).

These studies also demonstrate that SA IL-33 substantially reduces toxicity compared to wild type IL-33. Mice were induced with EAE at day 0 and received subcutaneous wild type or equimolar SA IL-33 every other day beginning at day 8. At day 11, after two treatments, mice receiving wild type IL-33 experienced severe toxicity and death (FIG. 30A). In mice receiving SA-IL-33, no death was observed, and mice continued to receive SA-IL-33 and were protected from the development of EAE (FIG. 30B). In healthy mice, dosing every other day for a total of 3 doses had little effect on production of serum IgE, compared to wild type IL-33 (FIG. 31A). After 5 doses, mice treated with SA-IL-33 had equivalent levels of serum IgE as those treated with wild type IL-33 (FIG. 31B). To determine if three doses was sufficient to protect from EAE, mice were induced with EAE and treated with SA-IL-33 every other day beginning on day 8 after induction. Three doses were sufficient to protect from EAE compared to 8 doses, suggesting that a three dose regimen would be sufficient to protect from disease (FIG. 31C-D).

### Example 9: SA-IL-33 for Treating Multiple Sclerosis (EAE)

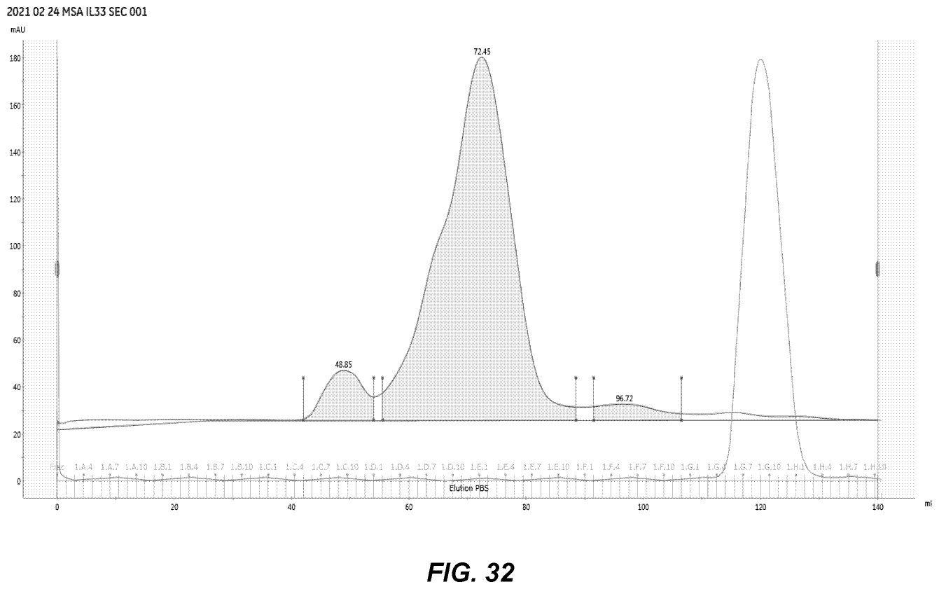

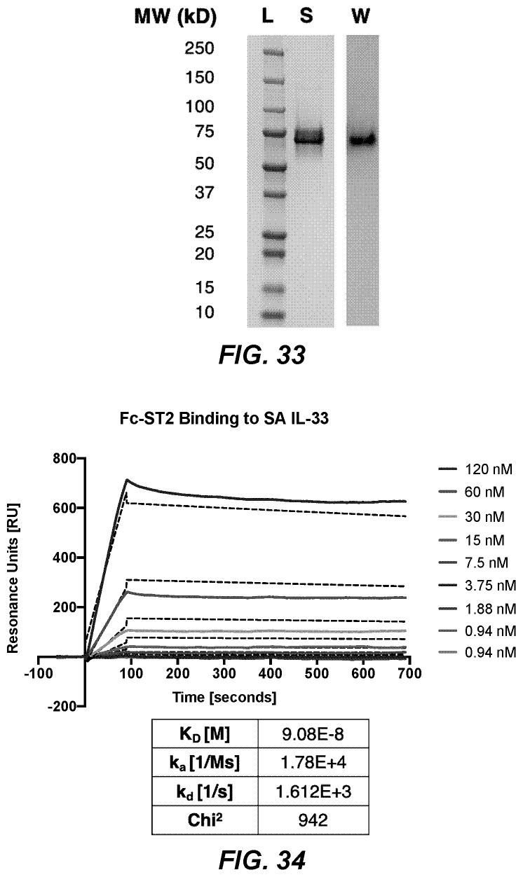

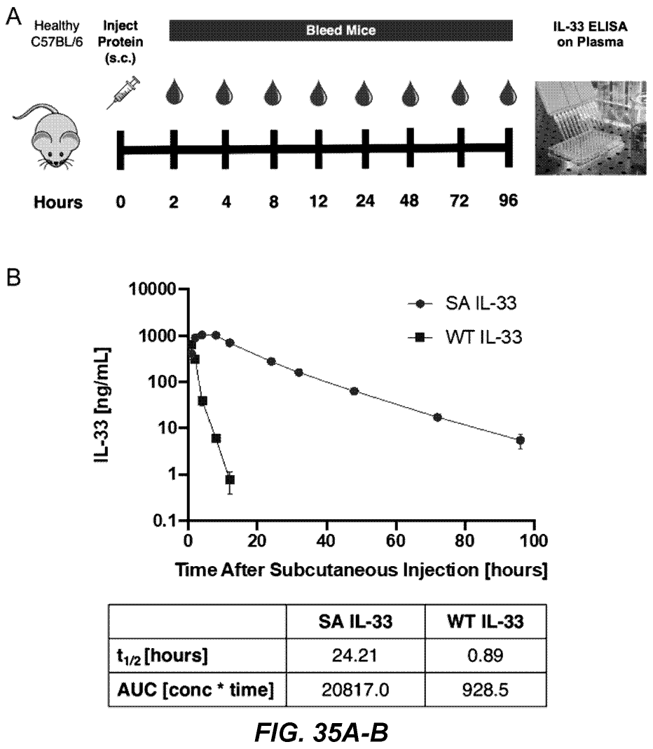

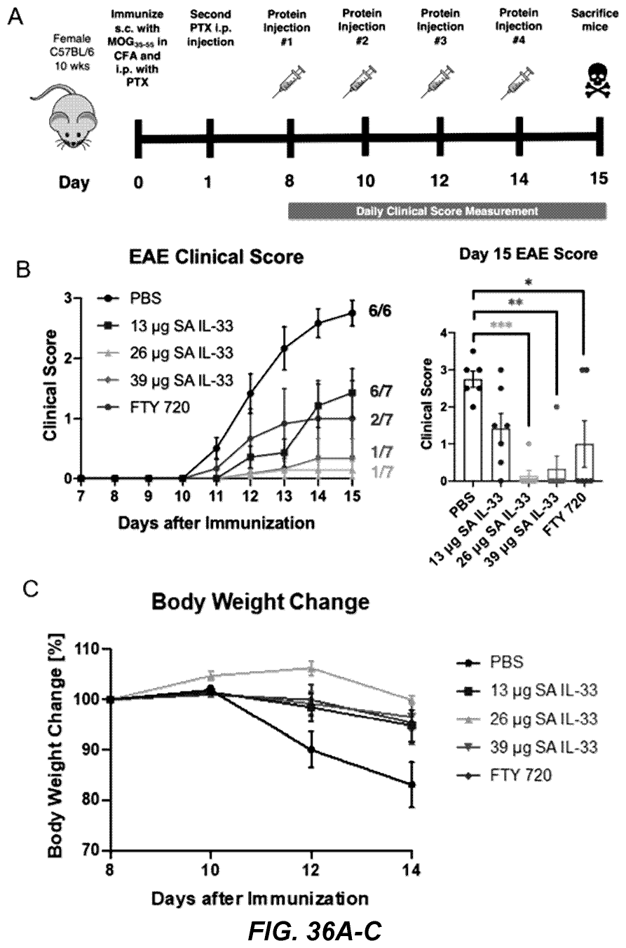

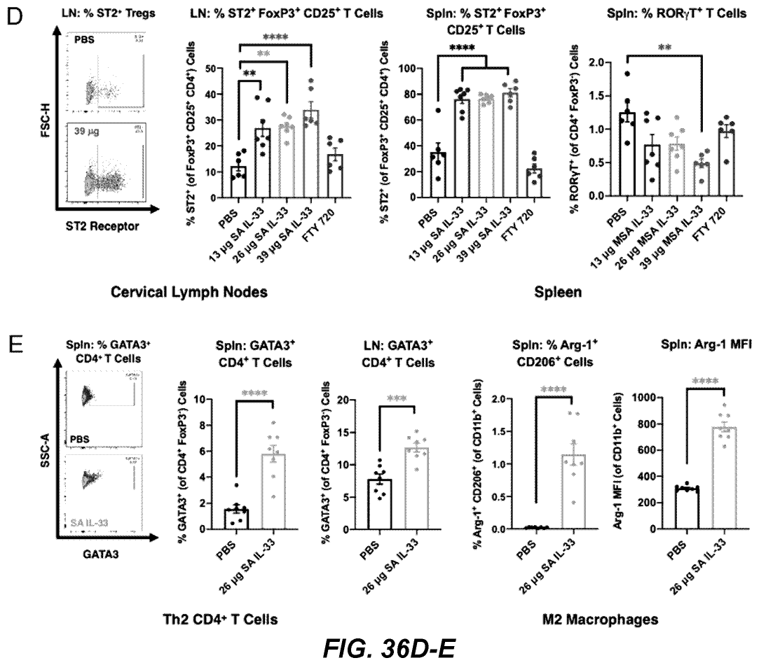

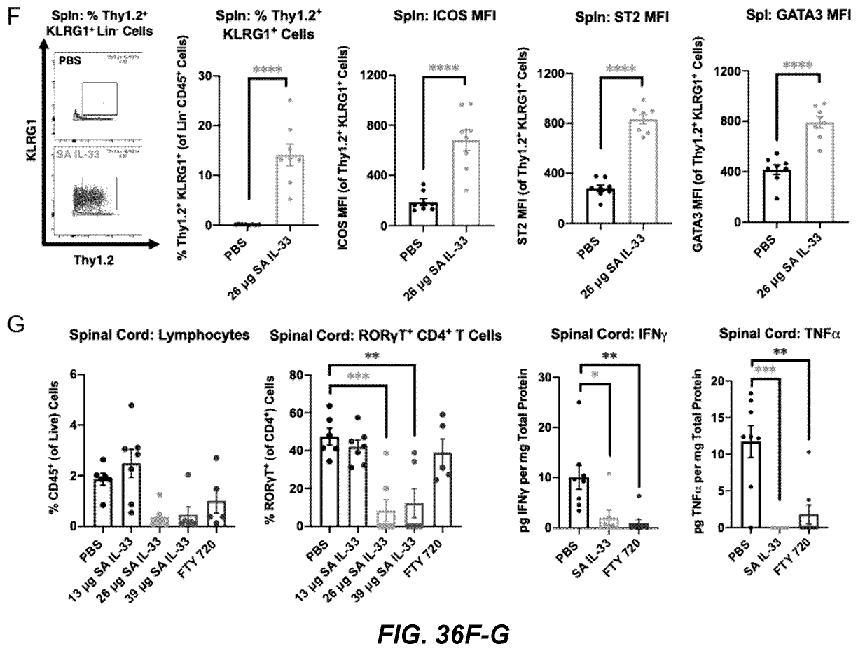

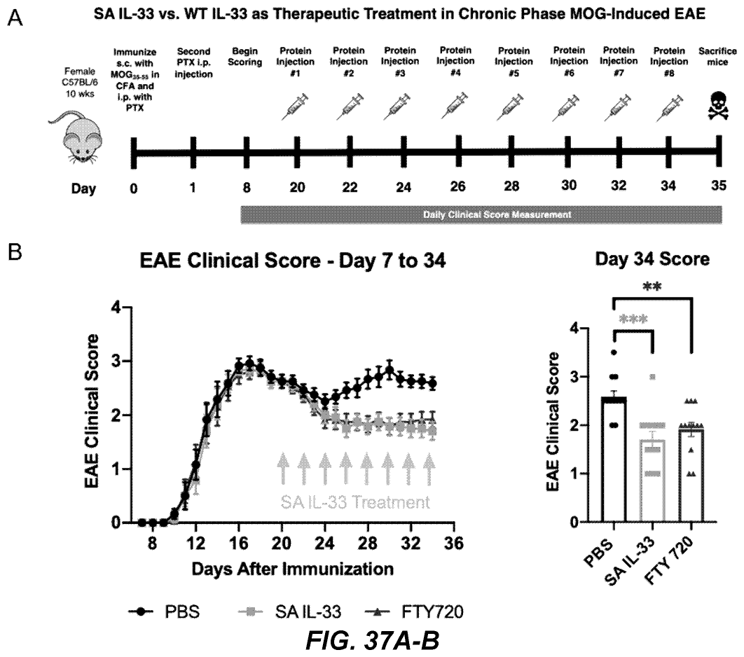

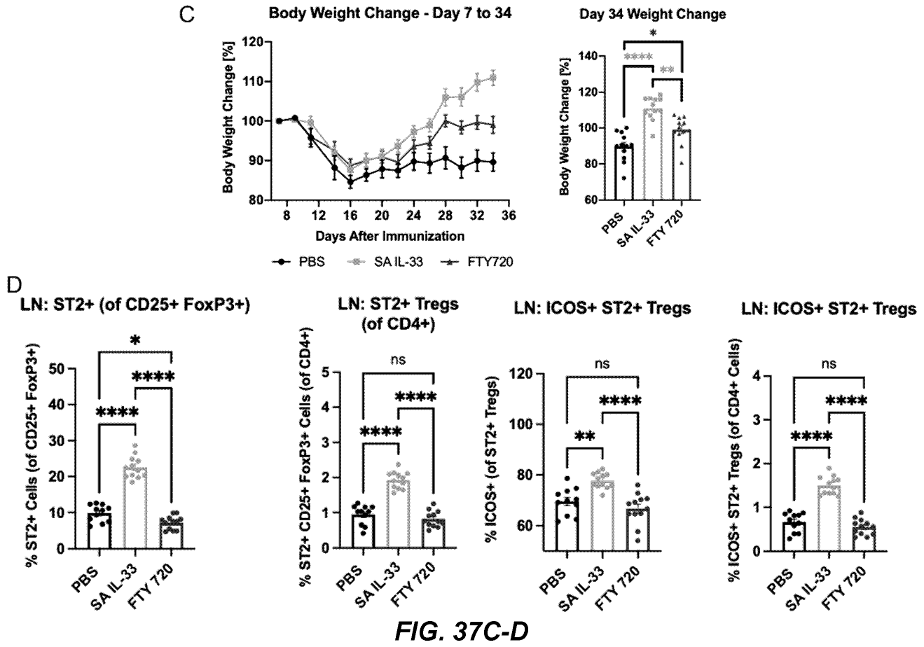

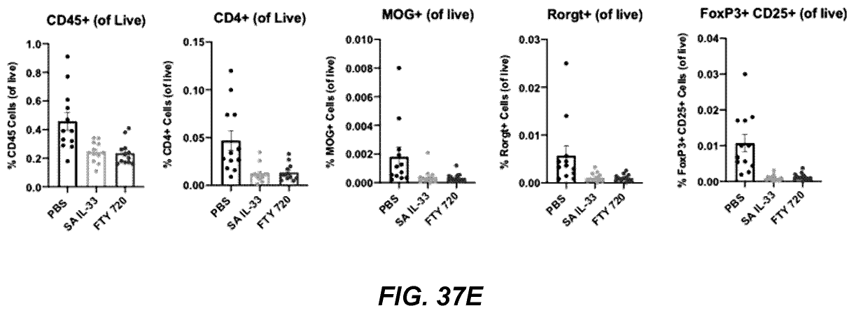

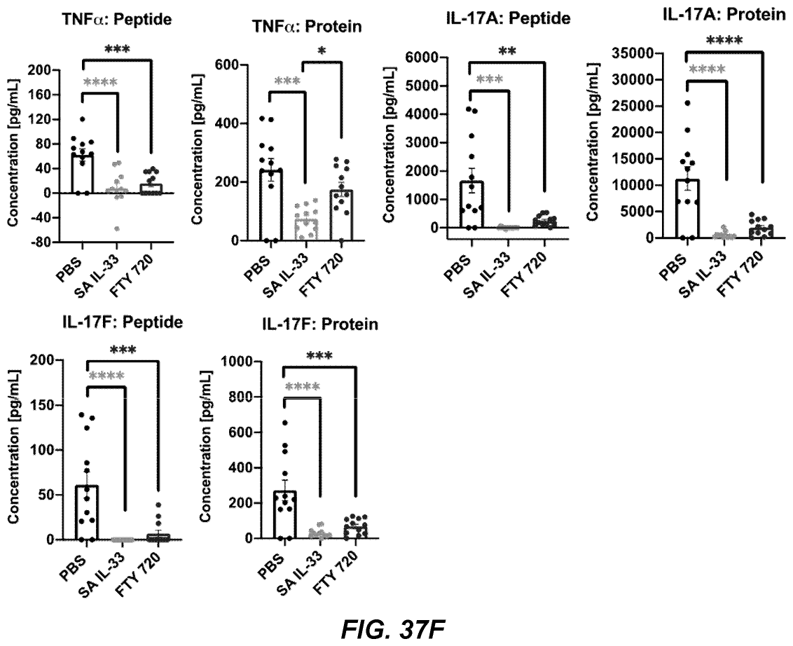

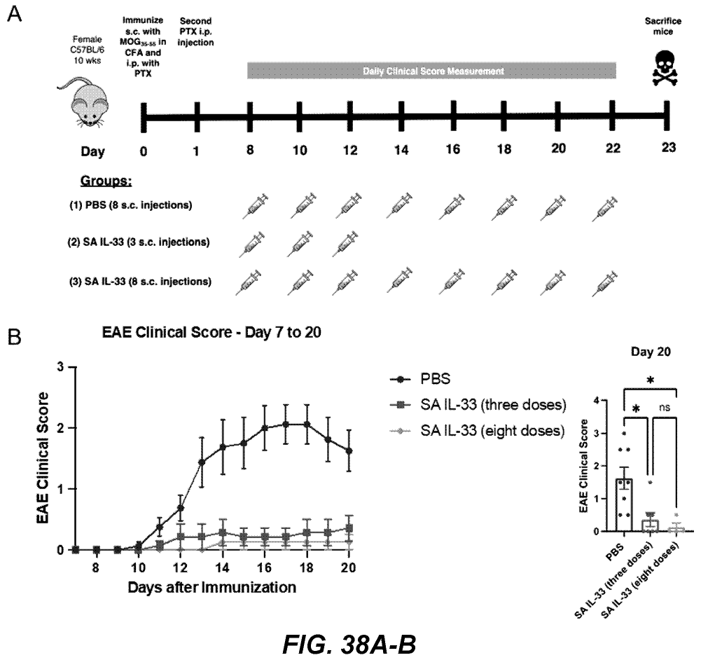

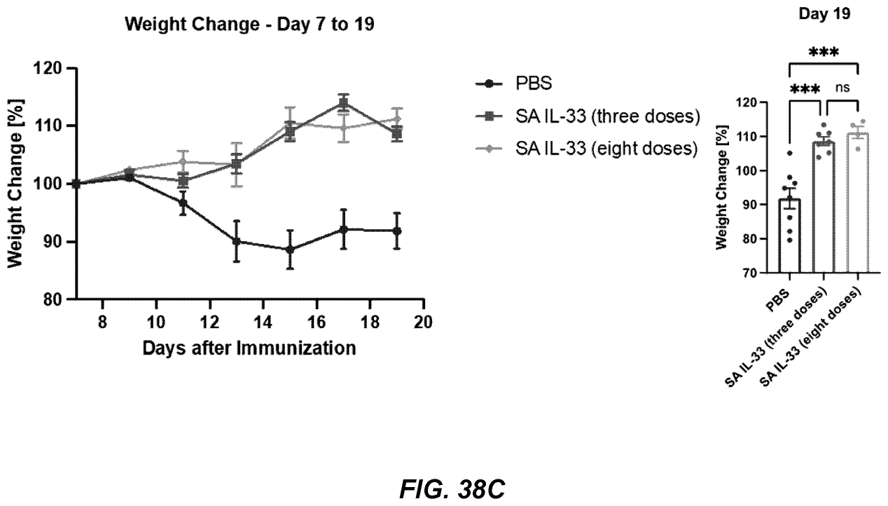

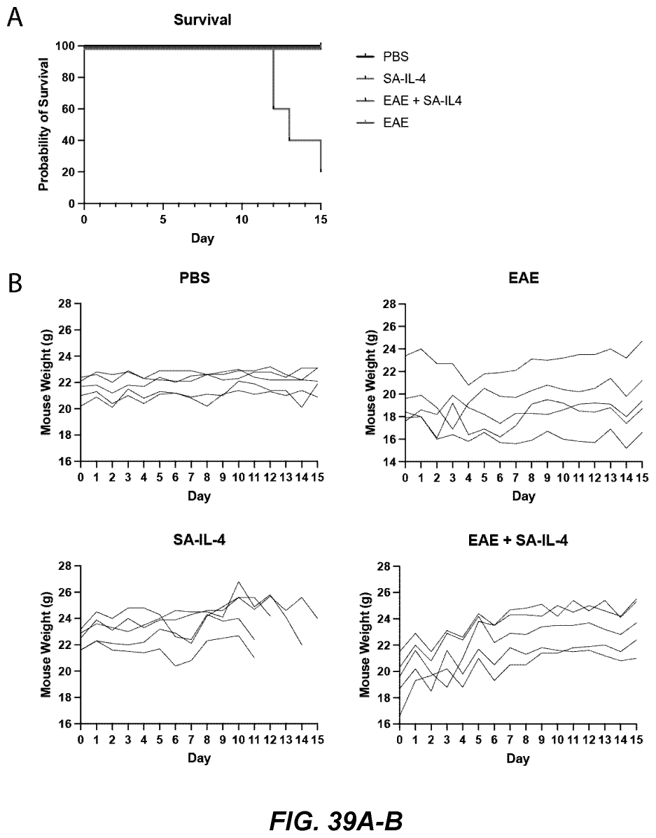

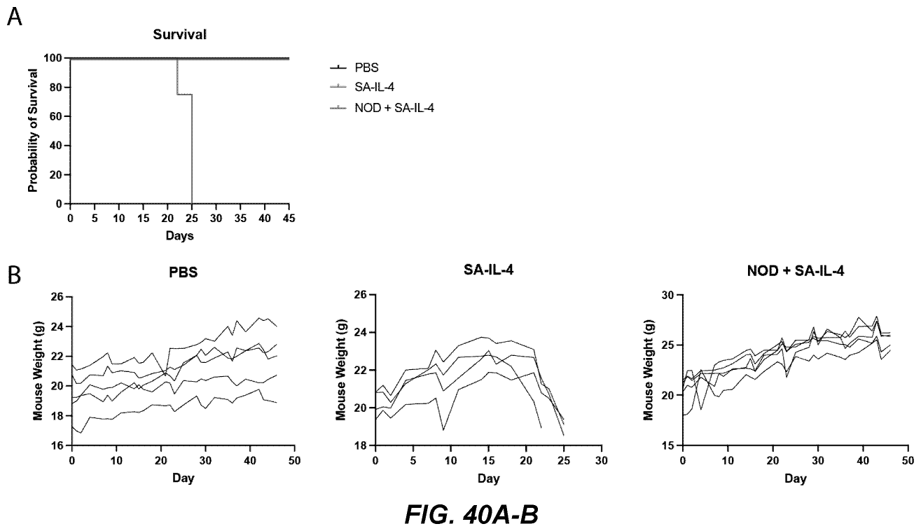

FIGS. 32-42 further demonstrate aspects of SA-IL-33 experiments in mice.

Methods

Production and purification of recombinant SA IL-33. The sequences encoding for mouse SA IL-33 without pro-peptide (25-608 amino acids of whole serum albumin), mouse IL-33 and a (GGGS)2 linker were synthesized and subcloned into the mammalian expression vector pcDNA3.1(+) by Genscript. A sequence encoding for 6-His was added at the carboxy terminus for further purification of the recombinant protein. Suspension-adapted HEK-293F cells were routinely maintained in serum-free FreeStyle 293 expression medium (Gibco). On the day of transfection, the cells were inoculated into fresh medium at a density of 1×10 cells ml; 2 μg ml plasmid DNA, 2 μg ml linear 25 kDa polyethylenimine (Polysciences) and OptiPRO SFM medium (4% final concentration; Thermo Fisher) were added sequentially. The culture flask was agitated by orbital shaking at 135 r.p.m. at 37° C. in the presence of 5% CO. Seven days after transfection, the cell culture medium was collected by centrifugation and filtered through a 0.22 m filter. The culture medium was loaded into a HisTrap HP 5 ml column (GE Healthcare) using an ÄKTA pure 25 (GE Healthcare). After washing the column with wash buffer (20 mM NaH2PO4 and 0.5 M NaCl, pH 8.0), protein was eluted using a gradient of 500 mM imidazole (in wash buffer). The protein was further purified by size-exclusion chromatography using a HiLoad Superdex 200PG column (GE Healthcare) with PBS as an eluent. All purification steps were carried out at 4° C. The expressed proteins were verified to be >90% pure through SDS-PAGE. The purified proteins were tested for endotoxin via the HEK-Blue TLR4 reporter cell line, and the endotoxin levels were confirmed to be below 0.01 EU ml-1. Protein concentration was determined through absorbance at 280 nm using a NanoDrop spectrophotometer (Thermo Scientific).

SPR. SPR measurements were made using a Biacore X100 SPR system (GE Healthcare). Biacore Sensor Chip Nitrilotriacetic acid (NTA) was immobilized with his-tagged 10 ug/mL SA IL-33 or his-tagged unmodified IL-33 until resonance units (RU) attained a value of 1000. A reference surface was coated with 1000 RU of 10 mg/mL his-tagged SA (no cytokine) as a control. Decreasing concentrations of the analyte Fc-ST2 ranging from 120 nM to 1.875 nM of the analyte at contact time of 60 seconds. Specific binding of SA IL-33 to Fc-ST2 was calculated by subtracting the SA-only coated reference channel values from the SA IL-33 values. The experimental results were then fitted with Langmuir binding kinetics using the BIAevaluation software (GE Healthcare).

Mice. C57BL/6 female mice (8 weeks old) were obtained from Charles River Laboratories. The mice were housed at the University of Chicago Animal Facility for at least 1 week before immunization. All experiments were performed with approval from the Institutional Animal Care and Use Committee of the University of Chicago.

Plasma pharmacokinetics of the proteins. Female C57BL/6 mice were given subcutaneous injections of WT IL-33 or SA-IL-33 (equivalent to 26 μg IL-33). Blood samples were collected in protein-low binding tubes at 2 hrs, 4 hrs, 8 hrs, 12 hrs, 24 hrs, 36 hrs, 48 hrs, 72 hrs, and 96 hrs after injection. The IL-33 concentrations in the plasma were measured using an IL-33 mouse uncoated ELISA kit (R&D Systems) according to the manufacturer's protocol.

EAE model. Young female C57BL/6 mice (9-12 weeks of age) were subcutaneously immunized at the dorsal flanks with an emulsion of MOG₃₅₋₅₅ in complete Freund's adjuvant (MOG₃₅₋₅₅/CFA Emulsion, Hooke Laboratories), followed by i.p. administration of pertussis toxin in PBS-first on the day of immunization and again the following day. After the first immunization, the severity of EAE was monitored and clinical scores were measured daily from day 8 after immunization. The clinical scores were determined based on the Hooke Laboratories criterium under blinding to the treatment grouping. WT IL-33, SA-IL-33 and PBS were administered s.c. (in the left back flank of the mouse; approximately 2 cm away from the emulsion injection site) in 200 μl PBS every other day. FTY720 (1 mg kg-1 body weight) was administered orally every day.

Flow cytometry. The EAE mice were treated with PBS, WT IL-33 (Biolegend) or SA IL-33 (equivalent to 26 μg IL-33) every other day, starting 8 d after immunization. The spinal cord, spleen and cervical & iliac LNs were harvested 15, 23 or 35 days after immunization. The lymph node and spinal cord tissues were digested in DMEM medium supplemented with 2% FBS, 2 mg ml-1 collagenase D (Sigma-Aldrich) for 45 min at 37° C. Single-cell suspensions were obtained by gentle disruption through a 70-μm cell strainer. For the spleen, red blood cells in the blood were lysed with ACK lysing buffer (Quality Biological), followed by antibody staining for flow cytometry. For detection of MOG-recognizing T cells, T-Select I-Ab MOG₃₅₋₅₅ tetramer-PE (MBL International Corporation) or MOG₃₈₋₄₉ tetramer-PE (NIH Tetramer Core Facility) were used. Non-specific binding of MOG₃₈₋₄₉ tetramer was not considered. Fixable live/dead cell discrimination was performed using Fixable viability dye eFluor 455 (eBioscience), Live/dead fixable violet (eBioscience) or Live/dead fixable aqua (eBioscience) according to the manufacturer's instructions. Staining was carried out on ice for 20 min. For intracellular staining, Cytofix/Cytoperm (BD Bioscience) was used to fix cells for 20 min at 4° C. For permeabilization, perm/wash buffer (BD Bioscience) was use and cells were stained in perm/wash buffer for 30 min at 4° C. Following a washing step, the cells were stained with specific antibodies for 20 min on ice before fixation. All flow cytometric analyses were done using a Fortessa (BD Biosciences) flow cytometer and analyzed using the FlowJo software (Tree Star).

Restimulation of splenocytes. Single-cell suspensions were created from the dLNs and spleens. For analysis of cytokine production, 5×10⁵ lymphocytes and 2×10⁶ splenocytes were plated in a 96-well round-bottom plate. The cells were stimulated with 10 μM MOG₃₅₋₅₅ peptide (Genscript). After 2 h, GolgiPlug (brefeldin A) and GolgiStop (Monensin) were added as per the manufacturer's protocol to block the secretion of intracellular cytokines. The cells were stained for flow cytometry 4 h later. For fixation, Cytofix/Cytoperm (BD Bioscience) was used for 20 min at 4° C. For permeabilization, perm/wash buffer (BD Bioscience) was used and the cells were stained in perm/wash buffer for 30 min at 4° C. For the 3 d restimulation, 2.5×10⁵ splenocytes were plated in a 96-well round-bottom plates. The cells were stimulated with 10 μM MOG₃₅₋₅₅ (Genscript) or 100 μg ml⁻¹ MOG protein (Anaspec). After 72 h, the supernatant was collected for analysis by LegendPlex Multiplex Cytokine Array (Biolegend).

Statistical analysis. Statistically significant differences between experimental groups were determined using the Prism software (v9, GraphPad). Where a one-way analysis of variance (ANOVA), followed by Tukey's HSD post hoc test was used, variance between groups was found to be similar using the Brown-Forsythe test. For single comparisons, a two-tailed Student's t-test was used. P values less than 0.05 were considered significantly different.

### Example 10: Albumin-Fused Human IL-35 Protein has Significant Therapeutic Effects on Arthritis

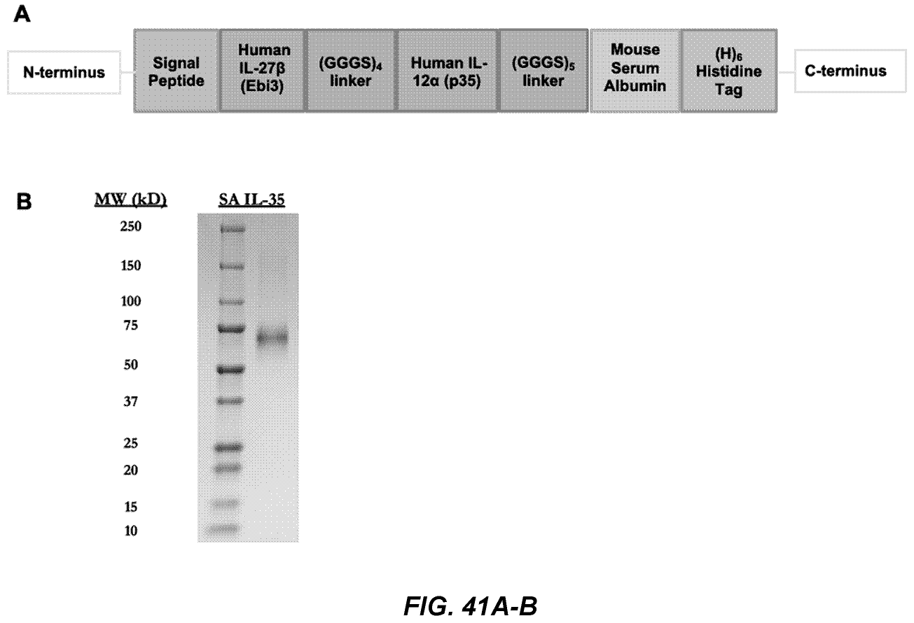

Serum albumin fused interleukin 35 (SA IL-35) was engineered by recombinantly fusing mouse SA to human IL-35. For this purpose, a single chain SA-IL-35 plasmid DNA construct consisting of starting from the N-terminus the IL-270 subunit of IL-35 (also known as Ebi3), a flexible (GGGS)₄ linker, the IL-12α subunit of IL-35 (also known as IL-12p35), the (GGGS)₅ flexible linker, mouse SA without pro-peptide, and a 6-histidine tag sequence along the C-terminus was synthesized and subcloned into the mammalian expression vector pcDNA3.1(+) as depicted in FIG. 1a. The protein was produced in suspension-adapted BEK-293F cells and purified via affinity and size exclusion chromatography. The molecular weight and purity of the protein were qualitatively evaluated via SDS PAGE. SDS-PAGE samples were stained with Commissar Blue. The SDS-PAGE of SA-IL-35 is depicted in FIG. 41B.

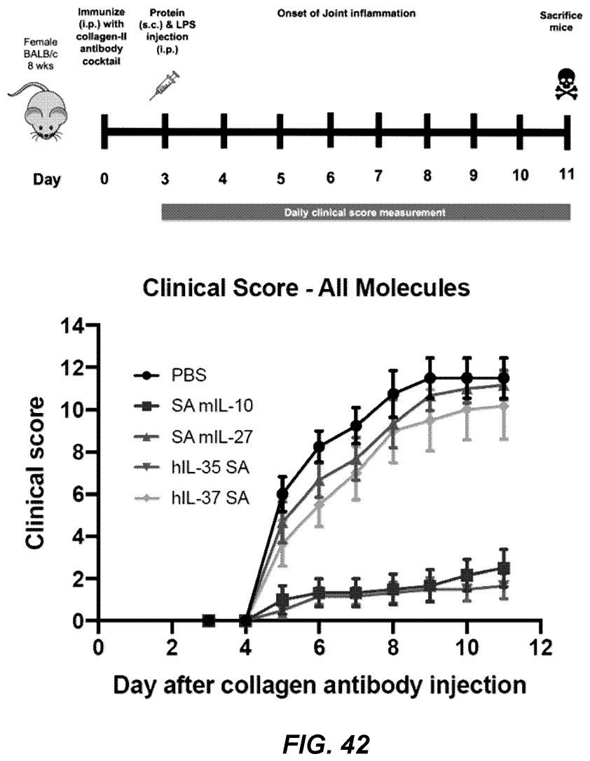

The inventors then evaluated SA IL-35 as a prophylactic treatment to prevent the onset of collagen antibody induced arthritis (CAIA). The CAIA model was induced, as depicted in FIG. 2a, on day 0 by intraperitoneally injecting the collagen-II antibody cocktail in BALB/c mice. On day 3 after collagen II antibody immunization, CAIA mice were treated with 43.5 ug SA IL-35 (wild type IL-35 molar equivalent) by intravenous injection, followed by an intraperitoneal injection of 25 mg LPS. From day 3 until day 11 the clinical score of the mice's front and hind paws were recorded daily. The severity of joint inflammation ranged from a score 0 to 4 where a score of 0 refers to healthy paw, 1 refers to swelling and/or redness in one joint, 2 refers to swelling and/or redness in more than one joint, 3 refers to swelling and/or redness in the entire paw, and 4 refers to maximal swelling. The results of the experiment are depicted below. Prophylactic treatment with a single dose of SA IL-35 prevented the onset of severe disease in the CAIA mouse model of arthritis. This data is shown in FIG. 42.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Friese, M. A., Schattling, B. & Fugger, L. Mechanisms of neurodegeneration and axonal dysfunction in multiple sclerosis. Nature reviews. Neurology 10, 225-238 (2014).
2. Ellwardt, E., Walsh, J. T., Kipnis, J. & Zipp, F. Understanding the Role of T Cells in CNS Homeostasis. Trends in immunology 37, 154-165 (2016).
3. Hofstetter, H. H. et al. Therapeutic efficacy of IL-17 neutralization in murine experimental autoimmune encephalomyelitis. Cellular Immunology 237, 123-130 (2005).
4. Chun, J. & Hartung, H. P. Mechanism of action of oral fingolimod (FTY720) in multiple sclerosis. Clinical neuropharmacology 33, 91-101 (2010).
5. Rice, G. P., Hartung, H. P. & Calabresi, P. A. Anti-alpha4 integrin therapy for multiple sclerosis: mechanisms and rationale. Neurology 64, 1336-1342 (2005).
6. Cooney, L. A., Towery, K., Endres, J. & Fox, D. A. Sensitivity and resistance to regulation by IL-4 during Th17 maturation. Journal of immunology (Baltimore, Md.: 1950) 187, 4440-4450 (2011).
7. Gadani, S. P., Cronk, J. C., Norris, G. T. & Kipnis, J. IL-4 in the brain: a cytokine to remember. Journal of immunology (Baltimore, Md.: 1950) 189, 4213-4219 (2012).
8. Racke, M. K. et al. Cytokine-induced immune deviation as a therapy for inflammatory autoimmune disease. The Journal of experimental medicine 180, 1961-1966 (1994).
9. Butti, E. et al. IL4 gene delivery to the CNS recruits regulatory T cells and induces clinical recovery in mouse models of multiple sclerosis. Gene therapy 15, 504-515 (2008).
10. Vogelaar, C. F. et al. Fast direct neuronal signaling via the IL-4 receptor as therapeutic target in neuroinflammation. Science translational medicine 10 (2018).
11. van Zwam, M. et al. Surgical excision of CNS-draining lymph nodes reduces relapse severity in chronic-relapsing experimental autoimmune encephalomyelitis. The Journal ofpathology 217, 543-551 (2009).
12. Dennis, M. S. et al. Albumin binding as a general strategy for improving the pharmacokinetics of proteins. The Journal of biological chemistry 277, 35035-35043 (2002).
13. Liao, W. et al. Priming for T helper type 2 differentiation by interleukin 2-mediated induction of interleukin 4 receptor alpha-chain expression. Nature immunology 9, 1288-1296 (2008).
14. Nilsen, J. et al. Human and mouse albumin bind their respective neonatal Fc receptors differently. Sci Rep 8, 14648-14648 (2018).
15. Andersen, J. T. et al. Single-chain variable fragment albumin fusions bind the neonatal Fc receptor (FcRn) in a species-dependent manner: implications for in vivo half-life evaluation of albumin fusion therapeutics. The Journal of biological chemistry 288, 24277-24285 (2013).
16. Andrews, R., Rosa, L., Daines, M. & Khurana Hershey, G. Reconstitution of a functional human type II IL-4/IL-13 receptor in mouse B cells: demonstration of species specificity. Journal of immunology (Baltimore, Md.: 1950) 166, 1716-1722 (2001).
17. Pierson, E. R., Stromnes, I. M. & Goverman, J. M. B cells promote induction of experimental autoimmune encephalomyelitis by facilitating reactivation of T cells in the central nervous system. Journal ofimmunology (Baltimore, Md.: 1950) 192, 929-939 (2014).
18. Rothhammer, V. et al. Th17 lymphocytes traffic to the central nervous system independently of a4 integrin expression during EAE. The Journal of experimental medicine 208, 2465-2476 (2011).
19. Sasaki, K. et al. IL-4 suppresses very late antigen-4 expression which is required for therapeutic Th1 T-cell trafficking into tumors. J Immunother 32, 793-802 (2009).
20. Ioannou, M. et al. Crucial role of granulocytic myeloid-derived suppressor cells in the regulation of central nervous system autoimmune disease. Journal of immunology (Baltimore, Md.: 1950) 188, 1136-1146 (2012).
21. Guenova, E. et al. IL-4 abrogates T(H)17 cell-mediated inflammation by selective silencing of IL-23 in antigen-presenting cells. Proceedings of the National Academy of Sciences of the United States ofAmerica 112, 2163-2168 (2015).
22. Lotfi, N. et al. Roles of GM-CSF in the Pathogenesis of Autoimmune Diseases: An Update. Frontiers in immunology 10 (2019).
23. Luna, G. et al. Infection Risks Among Patients With Multiple Sclerosis Treated With Fingolimod, Natalizumab, Rituximab, and Injectable Therapies. JAMA Neurology (2019).
24. De Angelis, F., John, N. A. & Brownlee, W. J. Disease-modifying therapies for multiple sclerosis. BMJ 363, k4674 (2018).
25. Comi, G. et al. Efficacy of fingolimod and interferon beta-lb on cognitive, MRI, and clinical outcomes in relapsing-remitting multiple sclerosis: an 18-month, open-label, rater-blinded, randomised, multicentre study (the GOLDEN study). J Neurol 264, 2436-2449 (2017).
26. Sanford, M. & Lyseng-Williamson, K. A. Subcutaneous recombinant interferon-beta-1a (Rebif(R)): a review of its use in the treatment of relapsing multiple sclerosis. Drugs 71, 1865-1891 (2011).
27. Barun, B. & Bar-Or, A. Treatment of multiple sclerosis with Anti-CD20 antibodies. Clinical Immunology 142, 31-37 (2012).
28. Taupin, P. Antibodies against CD20 (rituximab) for treating multiple sclerosis: US20100233121. Expert Opin Ther Pat 21, 111-114 (2011).
29. Apolloni, E. et al. Immortalized myeloid suppressor cells trigger apoptosis in antigen-activated T lymphocytes. Journal of immunology (Baltimore, Md.: 1950) 165, 6723-6730 (2000).
30. Crook, K. R. & Liu, P. Role of myeloid-derived suppressor cells in autoimmune disease. World journal of immunology 4, 26-33 (2014).
31. Komiyama, Y. et al. IL-17 Plays an Important Role in the Development of Experimental Autoimmune Encephalomyelitis. The Journal of Immunology 177, 566-573 (2006).
32. Lee, P. W. et al. IL-23R-activated STAT3/STAT4 is essential for Th1/Th17-mediated CNS autoimmunity. JCI Insight 2, e91663 (2017).
33. Liu, H. et al. Structure-based programming of lymph-node targeting in molecular vaccines. Nature 507, 519-522 (2014).
34. Wang, Y. et al. In vivo albumin labeling and lymphatic imaging. Proceedings of the National Academy of Sciences 112, 208-213 (2015).
35. Mirzaei, S. et al. Sentinel lymph node detection with large human serum albumin colloid particles in breast cancer. Eur J Nucl Med Mol Imaging 30, 874-878 (2003).
36. Yao, Z., Dai, W., Perry, J., Brechbiel, M. W. & Sung, C. Effect of albumin fusion on the biodistribution of interleukin-2. Cancer Immunol Immunother 53, 404-410 (2004).
37. Fan, Y. Y. et al. Human FcRn Tissue Expression Profile and Half-Life in PBMCs. Biomolecules 9 (2019).
38. Miyasaka, M. & Tanaka, T. Lymphocyte trafficking across high endothelial venules: dogmas and enigmas. Nature Reviews Immunology 4, 360-370 (2004).
39. Pyzik, M. et al. Hepatic FcRn regulates albumin homeostasis and susceptibility to liver injury. Proceedings of the National Academy of Sciences of the United States of America 114, E2862-e2871 (2017).
40. Pyzik, M. et al. The Neonatal Fc Receptor (FcRn): A Misnomer? Frontiers in immunology 10 (2019).
41. Hashem, L., Swedrowska, M. & Vllasaliu, D. Intestinal uptake and transport of albumin nanoparticles: potential for oral delivery. Nanomedicine (London, England) 13, 1255-1265 (2018).
42. Salou, M., Nicol, B., Garcia, A. & Laplaud, D.-A. Involvement of CD8(+) T Cells in Multiple Sclerosis. Frontiers in immunology 6, 604-604 (2015).
43. M. Miyasaka, T. Tanaka, Lymphocyte trafficking across high endothelial venules: dogmas and enigmas. Nat. Rev. Immunol. 4, 360-370 (2004).
44. M. Pyzik, et al., Hepatic FcRn regulates albumin homeostasis and susceptibility to liver injury. Proc. Natl. Acad. Sci. USA 114, E2862-E2871 (2017).
45. M. Pyzik, et al., The neonatal Fc receptor (FcRn): a misnomer? Front. Immunol. 10, 1540 (2019).
46. S. Kotake, T. Yago, T. Kobashigawa, Y. Nanke, The plasticity of Th17 cells in the pathogenesis of rheumatoid arthritis. J. Clin. Med. 6, E67 (2017).
47. J. P. van Hamburg, S. W. Tas, Molecular mechanisms underpinning T helper 17 cell heterogeneity and functions in rheumatoid arthritis. J. Autoimmun. 87, 69-81 (2018).
48. G. S. Firestein, Evolving concepts of rheumatoid arthritis. Nature 423, 356-361 (2003).
49. I. B. McInnes, G. Schett, The pathogenesis of rheumatoid arthritis. N. Engl. J Med. 365, 2205-2219 (2011).
50. Q. Guo, et al., Rheumatoid arthritis: pathological mechanisms and modern pharmacologic therapies. Bone Res. 6, 15 (2018).
51. M. E. Weinblatt, et al., Adalimumab, a fully human anti-tumor necrosis factor alpha monoclonal antibody, for the treatment of rheumatoid arthritis in patients taking concomitant methotrexate: The ARMADA trial. Arthritis Rheum. 48, 35-45 (2003).
52. Prevention CfDCa. 2020. Estimates of Diabetes and its Burden in the United States. National Diabetes Statistics Report: 1-32
53. Narres M ea. 2017. Incidence of lower extremity amputations in the diabetic compared with the non-diabetic population: A systematic review. PLOS ONE 12: 1-28
54. Roberts W G, Palade, G. E. 1995. Increased microvacular permeability and endothelial fenestration induced by vascular endothelial growth factor. Journal of Cell Science 108: 2369-79
55. Weis S M, Cheresh, D. A. 2005. Pathophysiological consequences of VEGF-induced vascular permeability. Nature 437: 497-504
56. Barman P K, Koh, T. J. 2020. Macrophage Dysregulation and Impaired Skin Wound Healing in Diabetes. Frontiers in Cell and Developmental Biology 8
57. Stein M, Keshav, S., Harris, N., Gordon, S. 1992. Interleukin 4 potently enhances murin macrophage mannose receptor activity: a marker of alternative immunologic macrophage activation. J. Exp. Med. 176: 287-92
58. Mantovani A, Sica, A., Sozzani, S., Allavena, P., Vecchi, A., Locati, M. 2004. The chemokine system in diverse forms of macrophage activation and polarization. Trends in Immunology 25: 677-86

1.-54. (canceled)

55. A method for promoting wound healing in a subject, the method comprising administering to the subject, by subcutaneous, intradermal, or intramuscular administration, an effective amount of a composition comprising an anti-inflammatory cytokine operatively linked to an albumin protein.

56.-90. (canceled)

91. A method for targeting an anti-inflammatory cytokine to a lymph node of a subject, the method comprising administering to the subject, by subcutaneous, intradermal, or intramuscular administration, a composition comprising the anti-inflammatory cytokine operatively linked to an albumin protein.

92. The method of claim 91, wherein the subject has an autoimmune or inflammatory condition.

93. (canceled)

94. The method of claim 92, wherein the autoimmune or inflammatory condition comprises MS, type 1 diabetes, diabetic peripheral neuropathy, psoriasis, inflammatory bowel disease, cytokine storm syndrome, systemic scleroderma, arthritis, rheumatoid arthritis, acute respiratory stress syndrome, or Crohn's disease.

95. The method of claim 91, further comprising identifying the anti-inflammatory cytokine in a lymph node of the subject.

96. The method of claim 95, wherein the identifying comprises obtaining a lymph sample from the subject.

97. (canceled)

98. The method of claim 91, wherein the anti-inflammatory cytokine remains in the lymph node at least eight hours after administering the composition to the subject.

99. The method of claim 91, wherein the anti-inflammatory cytokine remains in the lymph node at least sixteen hours after administering the composition to the subject.

100. The method of claim 91, wherein the anti-inflammatory cytokine is IL-4.

101. The method of claim 100, wherein the TL-4 operatively linked to the albumin protein is administered in a dose of 0.4-1.5 mg/kg.

102.-104. (canceled)

105. The method of claim 91, wherein the anti-inflammatory cytokine is IL-10.

106.-111. (canceled)

112. The method of claim 91, wherein the anti-inflammatory cytokine is IL-35.

113. The method of claim 91, wherein the anti-inflammatory cytokine is IL-36ra.

114. The method of claim 91, wherein the anti-inflammatory cytokine is IL-37.

115. The method of claim 91, wherein the anti-inflammatory cytokine is IL-38.

116. The method of claim 91, wherein the anti-inflammatory cytokine is interferon-β.

117. The method of claim 91, wherein the anti-inflammatory cytokine is TGF-β1.

118. The method of claim 91, wherein the albumin protein is human serum albumin.

119. The method of claim 91, wherein the anti-inflammatory cytokine is covalently linked to the albumin protein.

120. The method of claim 91, wherein the anti-inflammatory cytokine is covalently linked to the albumin protein via a linker.

121. The method of claim 91, wherein the anti-inflammatory cytokine is administered at a dose of between 0.1 mg/kg and 50 mg/kg.

122. A method for treating a subject for an autoimmune or inflammatory condition, the method comprising administering to the subject, by subcutaneous, intradermal, or intramuscular administration, an effective amount of a composition comprising an anti-inflammatory cytokine operatively linked to an albumin binding polypeptide.

123-125. (canceled)

126. The method of claim 122, wherein the subject is being treated with an immunotherapy.

127. The method of claim 122, wherein the immunotherapy comprises immune checkpoint blockade (ICB) therapy, adoptive T-cell therapy, cytokine therapy, CAR-T cell therapy, activation of co-stimulatory molecules, and combinations thereof.

128.-132. (canceled)
